(19) United States
(12) Patent Application Publication
(10) Pub. No.: US 2022/0288130 A1
(43) Pub. Date: Sep. 15, 2022

(54) COMPOSITIONS AND METHODS FOR DIAGNOSIS AND TREATMENT OF METABOLIC DISEASES AND DISORDERS

(71) Applicant: University of Louisville Research Foundation, Inc., Louisville, KY (US)

(72) Inventor: Huang-Ge Zhang, Louisville, KY (US)

(73) Assignee: University of Louisville Research Foundation, Inc., Louisville, KY (US)

(21) Appl. No.: 17/635,291

(22) PCT Filed: Aug. 14, 2020
(86) PCT No.: PCT/US2020/046348

§ 371 Date: Feb. 14, 2022

Related U.S. Application Data
(60) Provisional application No. 63/002,560, filed on Mar. 31, 2020.
(60) Provisional application No. 62/886,652, filed on Aug. 14, 2019.

(51) Int. Cl. A61K 35/38; A61K 9/127; A61K 9/51; G01N 33/92; A61K 36/9068; A61P 3/10; A61P 3/04

(52) U.S. Cl. CPC A61K 35/38; A61K 9/127; A61K 9/51; G01N 33/92; A61K 36/9068; A61P 3/10; A61P 3/04; G01N 2405/04; G01N 2800/04; G01N 2800/042

(57) ABSTRACT

Provided are methods for increasing insulin sensitivity in subjects. In some embodiments, the method include administering to the subject an effective amount of a composition comprising a lipid bilayer, wherein the lipid bilayer is low in total phosphatidylcholine (PC) or has been treated to reduce total PC. Also provided are methods for diagnosing insulin sensitivity and/or a metabolic-related disorders, for preferentially targeting hepatocytes, for preferentially targeting liver macrophages and/or monocytes, for inhibiting development of insulin resistance, optionally insulin resistance associated with diabetes, for restoring gut epithelial homeostasis, for enhancing expression of a Foxa2 gene product in cells, for inhibiting Akt-1-mediated inactivation of Foxa2 biological activities, for increasing expression of VAMP7, miR-375, or both in epithelial cells, for enhancing sorting of miR-375 from intestinal epithelial cells to exosomes, for inhibiting hepatic AhR expression, and for inhibiting development of obesity in subjects in need thereof.

### CROSS REFERENCE TO RELATED APPLICATION

This application claims the benefit of U.S. Provisional Patent Application Ser. No. 62/886,652, filed Aug. 14, 2019, and U.S. Provisional Patent Application Ser. No. 63/002,560, filed Mar. 31, 2020, the disclosure of each of which is incorporated herein by reference in its entirety.

### TECHNICAL FIELD

The presently disclosed subject matter relates generally to compositions and methods for diagnosis and treatment of metabolic diseases and disorders and inhibiting the development of the same. In some embodiments, the presently disclosed compositions and methods relate to methods for increasing insulin sensitivity in subjects in need thereof, methods for diagnosing insulin sensitivity and/or a metabolic-related disorders of the liver in subjects, methods for preferentially targeting hepatocytes in subjects, and/or methods for preferentially targeting liver macrophages and/or monocytes in subjects, which in some embodiments can involve administering to a subject in need thereof a ginger-derived nanoparticle (GDNP) in an amount and via a route sufficient to inhibit development of insulin resistance and/or obesity in the subject.

### BACKGROUND

The global escalation of obesity and diabetes poses a great health challenge. Insulin resistance is a hallmark of type 2 diabetes and is associated with metabolic disorders, yet the precise interplay between the molecular pathways that underlie is not fully understood. The accumulation of bioactive lipids in non-adipose tissues has been proposed to promote impaired insulin sensitivity. Abnormally high cellular phosphatidylcholine (PC) lipid influences energy metabolism and is linked to insulin resistance. Indeed, changes in the PC and/or phosphatidylethanolamine (PE) content of the liver are implicated in insulin resistance and obesity.

High-fat (HF) diets represent a public health concern as they can predispose individuals to obesity and diabetes and promote overproduction of PC and insulin resistance. From a physiological point of view, one of the most important links between the HF diet and insulin resistance is the gut—liver axis and the factors released from intestinal and liver metabolites which mediate a bidirectional communication between the intestines and the liver.

Obesity is a complex and chronic disease that affects more than a third of the world's population Changes in lifestyle, and particularly increased consumption of unhealthy diets, are thought to be major causes of the current epidemics of obesity and type 2 diabetics (T2D). This form of diabetes is characterized by insulin resistance. Given that among the numerous factors from a diet or dietary supplements that could contribute to modulate insulin signaling, identifying specific diet-derived factor(s) that contributes to modulate insulin signaling is challenging.

A number of diet-derived factors regulate the aryl hydrocarbon receptor (AhR) mediated signaling pathway which has been shown to regulate insulin response. Mice that express a low-affinity AhR allele were less susceptible to obesity after exposure to a HFD and exhibited differences in fat mass, liver physiology and hepatocyte gene expression compared to mice with high-affinity AhR. Serum AhR ligand levels were increased in T2D patient samples and correlated with measures of insulin resistance. However, the molecular mediators and mechanisms governing the association between diet, AhR and insulin pathway signaling in general are still elusive.

While AhR-mediated pathways promote the development of obesity, studies on mice and humans have suggested that chronic consumption of a HFD causes inactivation of the transcription factor Foxa2. Inactivation of a transcription factor, Foxa2, leads to the development of T2D. Moreover, a previous study found that Foxa2 expression is an important determinant in preventing disease onset and decreasing its severity. Foxa2 is essential for glucose and lipid homeostasis. Tissue-specific deletion of Foxa2 in pancreatic β cells in mice led to increased adiposity under a high-fat diet conditions and decreased adipocyte glucose uptake and glycolysis. Whether diet-derived factors regulate the activity of Foxa2 is not known. Furthermore, little is known about the relationship between AhR- and Foxa2-mediated pathways in terms of regulating the insulin response.

### SUMMARY

This Summary lists several embodiments of the presently disclosed subject matter, and in many cases lists variations and permutations of these embodiments of the presently disclosed subject matter. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter relates to methods for increasing insulin sensitivity in subjects in need thereof. In some embodiments, the methods comprise administering to a subject in need thereof an effective amount of a composition comprising a lipid bilayer, wherein the lipid bilayer is low in total phosphatidylcholine (PC) or has been treated to reduce total PC. In some embodiments, the composition is a nanoparticle or an exosome, optionally an intestinal exosome, and further optionally an intestinal exosome isolated from the subject. In some embodiments, the lipid bilayer of the composition has a total PC content that does not exceed about 14% lysophosphatidylcholine (LPC), about 10% ether-phosphatidylcholine (ePC), and/or about 10% PC as compared to total lipids.

In some embodiments, the presently disclosed subject matter also provides methods for diagnosing insulin sensitivity and/or a metabolic-related disorders of the liver in subjects. In some embodiments, the methods comprise assaying total phosphatidylcholine (PC) of intestinal exosomes isolated from a subject, wherein a total PC content of the intestinal exosomes isolated from the subject that is elevated relative to intestinal exosomes isolated from a normal subject is indicative of insulin sensitivity an/or a metabolic-related disorder of the liver in the subject.

In some embodiments, the presently disclosed subject matter also provides methods for identifying subjects with insulin sensitivity and/or a metabolic-related disorder of the liver. In some embodiments, the methods comprise assaying total phosphatidylcholine (PC) of intestinal exosomes isolated from a subject, wherein a total PC content of the intestinal exosomes isolated from the subject that is elevated relative to intestinal exosomes isolated from a normal subject is indicative of the subject having insulin sensitivity and/or a metabolic-related disorder of the liver. In some embodiments, total PC content of the intestinal exosomes isolated from the subject that exceeds about 14% lysophosphatidylcholine (LPC), about 10% ether-phosphatidylcholine (ePC), and/or about 10% PC as compared to total lipids is indicative of the subject having insulin sensitivity and/or a metabolic-related disorder of the liver.

In some embodiments, the presently disclosed subject matter also provides methods for preferentially targeting hepatocytes in subjects. In some embodiments, the methods comprise administering to a subject a composition comprising a lipid bilayer, optionally a nanoparticle, with a low total PC content and/or enhanced total phosphatidylethanolamine (PE) content, wherein the composition preferentially targets the subject's hepatocytes. In some embodiments, the total PE content of the lipid bilayer comprises PE of at least 50%, ether-phosphoethanolamine (ePE) of at least 30%, or both. In some embodiments, the composition is a nanoparticle or an exosome, optionally an intestinal exosome, and further optionally an intestinal exosome isolated from the subject. In some embodiments, the exosome is an intestinal exosome that has been treated to reduce the total PC content to less than about 35% and/or to enhance the total PE content to greater than about 35%.

In some embodiments, the presently disclosed subject matter also provides methods for preferentially targeting liver macrophages and/or monocytes in subjects. In some embodiments, the method comprising administering to a subject a composition comprising a lipid bilayer, optionally a nanoparticle, with a high total PC content and/or a reduced total PE content, wherein the composition preferentially targets the subject's liver macrophages and/or monocytes. In some embodiments. the total PE content of the lipid bilayer comprises PE of less than 35%, ether-phosphoethanolamine (ePE) of less than 30%, or both. In some embodiments, the composition is a nanoparticle or an exosome, optionally an intestinal exosome, and further optionally an intestinal exosome isolated from the subject. In some embodiments, the exosome is an intestinal exosome that has a total PC content greater than about 35% and/or a total PE content of less than about 35%.

In some embodiments, the presently disclosed subject matter also provides methods for inhibiting development of insulin resistance, optionally insulin resistance associated with diabetes, in a subject in need thereof. In some embodiments, the methods comprise, consist essentially of, or consist of administering to the subject a ginger-derived nanoparticle (GDNP) in an amount and via a route sufficient to inhibit development of insulin resistance in the subject. In some embodiments, the GDNP is administered to the subject orally. In some embodiments, the development of insulin resistance is incident to a high fat diet consumed by the subject.

In some embodiments, the presently disclosed subject matter also provides methods for restoring homeostasis in gut epithelium in a subject in need thereof. In some embodiments, the methods comprise, consist essentially of, or consist of administering to the subject a ginger-derived nanoparticle (GDNP) in an amount and via a route sufficient to restore homeostasis in gut epithelium in the subject.

In some embodiments, the presently disclosed subject matter also provides methods for enhancing expression of a Foxa2 gene product in a cell. In some embodiments, the methods comprise, consist essentially of, or consist of contacting the cell with a ginger-derived nanoparticle (GDNP) in an amount sufficient to enhance expression of the Foxa2 gene product in the cell.

In some embodiments, the presently disclosed subject matter also provides methods for inhibiting Akt-1-mediated inactivation of a Foxa2 biological activity in a subject. In some embodiments, the methods comprise, consist essentially of, or consist of administering to the subject a ginger-derived nanoparticle (GDNP) in an amount and via a route sufficient to inhibit Akt-1-mediated inactivation of a Foxa2 biological activity in the subject.

In some embodiments, the presently disclosed subject matter also provides methods for increasing expression of VAMP7, miR-375, or both in an epithelial cell, optionally an epithelial cell present in a subject. In some embodiments, the methods comprise, consist essentially of, or consist of contacting the epithelial cells with a ginger-derived nanoparticle (GDNP) in an amount sufficient to increase expression of VAMP7, miR-375, or both in the epithelial cell.

In some embodiments, the presently disclosed subject matter also provides methods for enhancing sorting of miR-375 from intestinal epithelial cells to exosomes. In some embodiments, the methods comprise, consist essentially of, or consist of contacting the intestinal epithelial cells with a ginger-derived nanoparticle (GDNP) in an amount sufficient to enhance sorting of miR-375 from the intestinal epithelial cells to exosomes. In some embodiments, the intestinal epithelial cells are present in a subject.

In some embodiments, the presently disclosed subject matter also provides methods for inhibiting hepatic AhR expression in a subject. In some embodiments, the methods comprise, consist essentially of, or consist of administering to the subject a ginger-derived nanoparticle (GDNP) in an amount and via a route sufficient to enhance sorting of miR-375 from intestinal epithelial cells to exosomes in the subject, whereby the exosomes are taken up by hepatocytes in the subject in an amount sufficient to inhibit hepatic applicants hereby reserve expression in the subject. In some embodiments, the presently disclosed subject matter also provides methods for inhibiting development of obesity in a subject in need thereof, In some embodiments, the methods comprise, consist essentially of, or consist of administering to the subject a ginger-derived nanoparticle (GDNP) in an amount and via a route sufficient to inhibit development of obesity in the subject.

In any of the presently disclosed methods, in some embodiments the subject or the cell is a human subject or a human cell. Thus, it is an object of the presently disclosed subject matter to provide compositions and methods for diagnosis and treatment of metabolic diseases and disorders.

An object of the presently disclosed subject matter having been stated herein above, and which is achieved in whole or in part by the presently disclosed subject matter, other objects will become evident as the description proceeds when taken in connection with the accompanying Figures as best described herein below.

### BRIEF DESCRIPTION OF THE FIGURES

FIGS. 1A-1D. HFD induced obesity and changes in mouse glucose regulation and liver physiology. FIG. 1A and 1B, Glucose tolerance (FIG. 1A) and insulin sensitivity (FIG. 1B) of RCD and HFD mice from which feces exosomes were isolated. FIG. 1C, Adiposity index (epididymal White Adipose Tissue (eWAT) to total body weight ratio of RCD and HFD mice (n=10 mice/group). FIG. 1D, Liver weight of RCD and HFD mice. Data are presented as the mean±SD. Student's t test, one-tailed. *<0.05; ***<0.001.

FIGS. 2A-2G. Isolation and characterization of intestinal exosomes. FIG. 2A, Representative image of sucrose gradient purification of H-Exo. The band at the interphase of 30% and 45% sucrose was used in downstream analysis. FIG. 2B, Electron microscopic images of exosomes from lean RCD mice (L-Exo) and HFD mice (H-Exo). FIG. 2C, The exosome size was estimated using a Malvern NanoSight NS300 (Malvern Instruments). FIG. 2D, Western blot images showing expression of CD63 (exosome marker) and A33 (intestinal epithelial cell marker) on both L-Exo and H-Exo exosomes. FIG. 2E, Exosomes were stained with A33 (green/FITC) and CD63 (red/PE) antibodies. Exosomes were analyzed by flow cytometry (left) and confocal microscopy (right). FIG. 2F, L-Exo and H-Exo yield per gram of mouse feces. Percentage positivity for CD63 was determined by NS300 (equipped with fluorescent channel). FIG. 2G, CD63⁺ exosomes were pulled down and the NS 300 (with fluorescent channel) was used to determine the percentage A33 positivity. Data are presented as the mean±SD. Student's t test, one-tailed. NS—non-significant; *<0.05; **<0.01.

FIGS. 3A-3H. Protein and miRNA characterization of intestinal exosomes. FIG. 3A, Proteins derived from fecal exosomes (CD63⁺A33⁺) from mice fed either RCD or HFD for 12 months were analyzed by SDS-PAGE (left) and a heat map for proteins detected by MS/MS analysis was generated (right). FIG. 3B, Bioanalyzer RNA profile and scatter plots for miRNAs (Qiagen miRNA array) from fecal exosomes (CD63⁺A33⁺) from mice fed either RCD or HFD for 12 months. FIG. 3C, The size of exosomes isolated from healthy control and type 2 diabetic (T2D) humans was estimated using the NS300. FIG. 3E, Western blot images confirming expression of CD63 and A33 in human fecal exosomes. FIGS. 3E and 3F, Healthy control and T2D-derived exosomes were stained with A33 (green/FITC) and CD63 (red/PE) antibodies and analyzed by flow cytometry (FIG. 3E) and confocal microscopy (FIG. 3F). FIG. 3G, Exosome yield was determined per gram of human feces. FIG. 3H, Percentage positivity for CD63 determined using the NS300 (equipped with fluorescent channel). Data are presented as the mean±SD. Student's t test, one-tailed. NS—non-significant; **<0.01.

FIGS. 4A-4C. High-fat diet (HFD) alters the lipid composition of intestinal epithelial cell-released exosomes. FIG. 4A, Immunoblots showing PEMT protein levels in mouse intestinal tissue extracts after mice were fed either a RCD or HFD for 3, 6, or 12 months. FIG. 4B, Mouse hepatocytes (FL83B) were transfected with pGL3B-PEMT-luc and treated with fecal metabolites from mice fed either a RCD or HFD for 12 months. Normalized luciferase activity was measured as an indication of PEMT expression. FIG. 4C, HPLC analysis of PE and PC in human fecal exosomes from T2D patients and healthy controls. Student's t test (one-tailed) or one-way ANOVA with a Bonferroni post hoc test. *<0.05; **<0.01; ***<0.001.

FIGS. 5A and 5B, Visualization of GFP-labeled exosomes by confocal microscopy in sections from the spleen (FIG. 5A) and MLN (FIG. 5B) of mice injected with GFP-MC38 cells (0.5×10⁶ cells) into the colon.

FIGS. 6A and 6B. CD63⁺A33⁺ exosomes traffic to the liver. FIG. 6A, Live imaging of mice orally gavaged with DIR-labelled exosomes at different time intervals. FIG. 6B, Scanned images of preferential localization of both L-Exo and H-Exo to the liver.

FIGS. 7A-7F. CD67⁺A77⁺ exosome uptake by liver cells. FIG. 7A, Confocal images of GFP-positive exosomes detected in mouse liver after injection of GFP-MC38 epithelial cells into the colon. DAPI was used to stain the nucleus. FIG. 7B, Flow cytometry analysis of PKH-26-labeled exosome uptake by hepatocytes (Albumin⁺) and Kupffer cells (F4/80⁺). FIG. 7C, PKH26-labeled exosomes visualized by confocal microscopy in hepatocytes/albumin⁺/green (arrows in Albumin/PKH26 Exo sections in upper panel) and Kupffer cells/F4/80/purple (arrows in F4/80/PKH26 Exo sections in upper panel). The percentages of total exosome uptake per cell type are summarized in the bar graph in the lower panel. FIG. 7D and FIG. 7E, PKH-26-labeled nanoparticles were cultured with hepatocytes (FIG. 7D) and monocytes (FIG. 7E). Cells were analyzed by flow cytometry and the percentage of PKH-26 positive cells was assessed after treatment with each nanoparticle (summarized in the bar graphs of FIG. 7D and FIG. 7E). FIG. 7F, Glucose uptake assays performed on mouse hepatocytes (FL83B cells) and human hepatocytes (HepG2 cells) cultured with CD63⁺A33⁺ mouse exosomes (L-Exo and H-Exo), nanoparticles generated from total lipids, PC depleted and added lipids of exosomes, and human exosomes for 16 hours. One-way or two-way ANOVA test. NS—non-significant; *<0.05; **<0.01; ***<0.001.

FIGS. 8A-8C. Preferential localization of L-Exo versus H-Exo to hepatocytes. FIGS. 8A and 8B, In vitro uptake of PKH26-labelled L-Exo or H-Exo by mouse hepatocytes (FL83B cells) with accompanying 3D images (FIG. 8A) and by human hepatocytes (HepG2 cells) (FIG. 8B). FIG. 8C, In vitro uptake of PKH26-labelled L-Exo or H-Exo by hepatocytes (FL83B cells) vs monocytes (U937 cells).

FIGS. 9A-9E. Crosstalk between hepatocytes and macrophage cells contributes to insulin resistance. FIG. 9A, Fold change in H-Exo vs L-Exo-induced cytokine expression for all cytokines showing >2-fold change. Red bars show cytokines/factors known to be involved in insulin resistance. FIG. 9B, Macrophage (MQ) depletion by single injection of CLODROSOME® brand liposomal clodronate (Encapsula Nanosciences LLC, Nashville, Tenn., United States of America) as assessed by flow cytometry for whole blood staining of F4/80. FIG. 9C, bar graph of fold changes in H-Exo vs L-Exo-induced cytokine levels in mouse plasma with or without macrophage depletion (MQ-). FIG. 9D, Glucose uptake assay performed on mouse hepatocytes supplemented with supernatant derived from macrophages cultured with nanoparticles derived from H-Exo total lipids (H-Exo Nano) and PC (34:2). FIG. 9E, Supernatants from H-Exo treated macrophages (monocytes+5×10⁶) were pre-neutralized with anti-TNF-α and/or anti-IL-6 antibodies. Glucose uptake by hepatocytes cultured in the presence of pre-neutralized supernatant was estimated. Data are presented as the mean±SD. One-way ANOVA with a Bonferroni post hoc test. NS—not significant; *<0.05; **<0.01; ***<0.001.

FIGS. 10A-10J. HFD-induced CD63⁺A33⁺ exosomal lipids contribute to insulin resistance in an AhR-dependent manner. FIG. 10A, Representative gene expression heat map for the Affymetrix array of liver tissue from mice orally administered exosomes for 14 days. Induction of AhR expression highlighted by red box. Elevated AhR expression was confirmed by qPCR (bar graph, right). FIG. 10B, Phosphorylated AhR (pAhR) protein expression in hepatocytes (FL83B cells) cultured with L-Exo, H-Exo, L-Exo^{nano}, or H-Exo^{nano}. FIG. 10C, FL83B cells were cultured with different concentrations (as indicated) of PC (34:2) for 16 hours, and the resulting effects on AhR expression was determined by western blots. FIGS. 10D and 10E, SPR sensogram showing the interaction of AhR recombinant protein with nanoparticles derived from total lipids of H-Exo (FIG. 10D and PC (34:2)^{nano} (FIG. 10E). FIG. 10F, PC direct binding to AhR protein. FIG. 10G, SPR was performed with AhR protein coated onto NTA chip and H-Exo^{nano PC-} and PC (34:2)^{nano} run over as mobile phase. FIG. 10H, pAhR expression in the cytoplasm vs nucleus of mouse hepatocytes cultured with L-Exo or H-Exo. Densitometry analysis of cytoplasmic (left) vs nuclear (right) pAhR protein expression following treatment with L-Exo or H-Exo. FIGS. 10I and 10J, Glucose uptake assay performed on wild-type FL83B (FIG. 10I) and AhR knockout (AhRKO; FIG. 10J) FL83B cells. Data represent the mean±SD. One-way ANOVA with a Bonferroni post hoc test. NS—non-significant; *<0.05; **<0.01; ***<0.001.

FIGS. 11A-11D. Gene expression in treated hepatocytes. FIG. 11A, Gene expression heat map from the insulin-signaling array of hepatocytes treated with L-Exo and H-Exo. FIG. 11B, Up- or downregulated genes in hepatocytes treated with L-Exo^{nano}, H-Exo^{nano} and PC (34:2)^{nano} confirmed by qPCR. FIG. 11C, IRS-2 mRNA expression in AhR knockout hepatocytes treated with L-Exo or H-Exo. FIG. 11D, Glucose uptake assay performed on mouse hepatocytes overexpressing IRS-2 and cultured with L-Exo or H-Exo. Data are presented as the mean±SD. One-way ANOVA with Bonferroni post hoc test. NS—non-significant; *<0.05; ***<0.001.

FIG. 12. H-Exo induced dyslipidemia in C57BL/6 and C57BL/6 germ-free mice, but not in AhR^{-/-} mice. Oral administration of H-Exo lead to significantly elevated the level of ALT and AST in mice plasma. Data are presented as the mean±SD. One-way ANOVA with a Bonferroni post hoc test. *<0.05; **<0.01.

FIGS. 13A-13E. Characterization of ginger nanoparticles (GDNP). FIG. 13A. Depiction of the sucrose gradient purification of ginger-derived nanoparticles (GDNP). FIG. 13B. Electron micrograph of purified GDNP from red box in FIG. 13A. FIG. 13C. GDNP size distribution, as determined by Nano-sight NS300. FIG. 13D. LPS detection in GDNP. Student t (one-tailed) test . p value *<0.05. FIG. 13E. Thin layer chromatography (TLC) profile of lipids derived from whole ginger root (GT) and ginger nanoparticles (GDNP) stained with CuSO₄ and iodine. The box shows the lipid band 1, responsible for induction of Foxa2.

FIGS. 14A-14N: Ginger-derived nanoparticles (GDNP) inhibit the phosphorylation of Foxa2 in intestinal epithelial cells. FIG. 14A. PKH-26 (red)-labeled GDNP uptake by small intestine epithelial (A33 positive/green) cells as shown by confocal 3D imaging. Enlarged image of cells containing labeled GDNP (PKH26/red) shown by red arrows. FIG. 14B. Representing the alteration of genes expression by affymetrix array of small intestine (SI) tissues from high-fat diet (HFD)-fed mice treated with either PBS or GDNP. Red boxes highlighted the genes involved in insulin signaling and lipid metabolism. FIG. 14C. Normalized (to β-actin) qRT-PCR quantification of Foxa2 mRNA expression in the mouse small intestine (SI) and large intestine (LI). FIG. 14D. Confocal images of frozen sections of the small intestine showing Foxa2 expression (green) and DAPI for nucleus staining (blue). FIG. 14E. Western blot representing total Foxa2 expression in mouse small intestine tissues. FIG. 14F. Corresponding densitometry analysis of the western blot for Foxa2 protein expression (expressed as the ratio to β-actin expression). FIG. 14G. Upregulation of Foxa2 mRNA (bar graphs, left panel) and protein (western blot, right panel) expression in GDNP-treated mouse colon (MC-38) and human colon (Caco2) cell lines. The ratio to β-actin shown in the middle (numbers). FIG. 14H. Visualization of Foxa2 (green) expression in MC-38 cells cultured with different lipid nanoparticles or complete GDNP. DAPI was used for nuclear staining. LB1-, lipid band 1 depleted; PC, phosphatidylcholine; PA, phosphatidic acid; LysoPG, lysophosphatidyl glycerol; GDNP, ginger-derived nanoparticles. FIG. 14I. qPCR quantification of Foxa2 mRNA in MC-38 cells cultured with different lipid nanoparticles or complete GDNP, as in FIG. 14H. FIGS. 14J and 14K. The surface plasmon resonance (SPR) sensogram representing the interaction between GDNP lipid nanoparticles (FIG. 14J) and phosphatidic acid (PA 18:1) nanoparticles (FIG. 14K) with recombinant Foxa2 protein. FIG. 14L. The SPR sensogram represents the interaction between PA (18:1) nanoparticles and the CRM1 and T156 Foxa2 synthesized peptide sequences. FIG. 14M. Western blot of phosphorylated Foxa2 (pFoxa2) expression in small intestine tissue derived from lean and HFD mice. The ratio to β-actin shown in the middle (numbers). FIG. 14N. Western blot of nuclear vs cytoplasmic levels of Foxa2 in MC-38 cells treated with PBS or GDNP. The ratio to histone for nuclear expression of Foxa2 shown in the right. One-way ANOVA with the Bonferroni correction for multiple comparisons and student t test (one tailed) were used to calculate statistical significance (p value *<0.05; **<0.01; ***<0.001).

FIGS. 15A and 15B. GDNP-derived lipids upregulate Foxa2 expression. FIG. 15A. Foxa2 protein expression in MC-38 cells following treatment with PBS, GDNP RNA, protein extracts with phosphatidic acids (PAs) lipids or GDNP. FIG. 15B. Foxa2 mRNA expression following treatment with either PBS, each band of GDNP lipids, or complete GDNP. One-way ANOVA with a Bonferroni correction for multiple comparisons was used to calculate statistical significance. (p value *<0.05; **<0.01; ***<0.001).

FIGS. 16A-16K: Foxa2-induces miR-375 expression by binding to miR-375 promoter. FIG. 16A. Graphical representation of the locations of Foxa2 binding sites in the miR-375 promoter and the initiation site for miR-375 transcription. The blue arrow shows the primers used in the cloning of the miR-375 promoter. FIG. 16B. ChIP assay showing Foxa2 binding to the miR-375 promoter. L, ladder; C, template used as the pulldown product by the control antibody (IgG) and F, template used as the pulldown product by the Foxa2 antibody. FIG. 16C. Heat map representing the microRNA array performed on wild type (WT) and Foxa2 knockout (Foxa2KO) MC-38 cells treated with PBS or GDNP. miR-375 is outlined by the red box (CT, threshold cycle). FIG. 16D. Bar graph showing the fold-change in the expression of microRNAs in WT MC-38 cells induced by GDNP treatment, (expression following treatment with PBS served as the baseline value). The Bar graph shows only microRNAs with >5 fold up- or downregulation. FIG. 16E. Normalized (subtracted background) luciferase activity (RLU, relative luminescence unit) measured after transfection of the miR-375 promoter cloned in the pGL3 vector (pGL3miR375) into WT and Foxa2KO MC-38 cells. FIG. 16F. Normalized luciferase activity measured in pGL3miR375-transfected WT and Foxa2KO MC-38 cells treated with PBS or GDNP. FIG. 16G. Graphical representation of the mutations created in the pGL3miR375 sequence 5′-ATGAGTCAATA-3′ (SEQ ID NO: 32) by site-directed mutagenesis to 5′-ACAAGCCAACG-3′ (SEQ ID NO: 33) in the Foxa2 binding site within the miR-375 promoter region. FIG. 16H. Normalized luciferase activity measured in MC-38 cells treated with pGL3miR375 or the mutated construct (Mut-pGL3miR375). FIG. 16I. Quantification of miR-375 expression in WT and Foxa2KO MC-38 cells treated with either PBS or GDNP. FIG. 16J. Intracellular levels of miR-375 in MC-38 cells treated with GDNP at the indicated time points. Significance was determined compared with time zero. FIG. 16K. Intracellular vs extracellular levels of miR-375 in MC-38 cells cultured with various concentrations of GDNP. One-way ANOVA with the Bonferroni correction for multiple comparisons and Student t test (one tailed) were used to calculate statistical significance (p value *<0.05; **<0.01; ***<0.001).

FIG. 17: Cloning strategy for the miR-375 promoter region into the pGL3 promoter vector and replacing the SV40 promoter.

FIGS. 18A-18Q: miR-375 sorted into exosomes via VAMP7, and intracellular miR-375 regulates AhR expression. FIG. 18A. Graphical representation of miR-375 binding site 5′-AGUGCGCUCGGUUGCUUGUUU-3′ (SEQ ID NO: 34) in AhR mRNA (3′-UUGUAUAGAUAUAAUGAACAAA-5′; SEQ ID NO: 35). FIG. 18B. qPCR quantification of AhR expression in the small intestine tissues of HFD fed mice treated with PBS or GDNP. FIG. 18C. Western blot presenting AhR expression in the small intestine tissues of lean and HFD-fed mice treated with PBS or GDNP. FIGS. 18D and 18E. AhR mRNAby qPCR (FIG. 18D) and protein expression by western blot (FIG. 18E) in miR-375 transfected MC-38 cells. FIGS. 18F and 18G. AhR mRNA (FIG. 18F) and protein expression (FIG. 18G) in the small intestine tissues of RCD mice orally administered nanoparticles (Nano-scramble or Nano-miR375). FIG. 18H. Intracellular and exosomal miR-375 levels, intracellular AhR mRNA levels by qPCR and western blots for VAMP7 protein levels in MC-38 cells cultured with various concentrations of GDNP. FIG. 18I. miRNA array expression profile of intestinal epithelial cell exosomes (A33+CD63+ exosomes) from feces derived from HFD-fed mice treated with GDNP vs PBS. FIG. 18J. Bar graph showing HFD-fed mouse fecal exosomal miRNAs with a fold change (>25-fold or <5-fold) following treatment with GDNP vs PBS. FIG. 18K. qRT PCR analysis of miR-375 expression in intestinal epithelial cell- exosomes (A33+CD63+) from lean and HFD-fed mice treated with GDNP vs PBS. FIG. 18L. Western blot showing VAMP7 expression in the small intestine of lean and HFD-fed mice treated with PBS or GDNP. FIG. 18M. qPCR (left) and western blot (right) analysis of VAMP7 expression in PBS- or GDNP-treated WT and Foxa2KO MC-38 cells. FIG. 18N. Confocal images displaying VAMP7 expression in PBS- or GDNP-treated MC-38 cells. FIG. 18O. qPCR analysis of the intracellular expression of miR-375 in WT and VAMP7KO MC-38 cells. FIG. 18P. qPCR analysis of miR-375 levels in exosomes released harvested from WT or VAMP7KO MC-38 cells. FIG. 18Q. MC-38 cells were transfected with biotinylated miR-375 and pulled-down with streptavidin beads. Western blot was carried out for VAMP7 by using eluted extract from streptavidin beads.

FIGS. 19A-19M: Gut epithelial cell exosomes (CD63+A33+) influences the gut bacterial populations and modulate microbial metabolites. FIG. 19A. Representative electron micrograph of gut bacteria containing fecal exosomes. Yellow arrows indicate exosomes inside and outside the bacteria. FIG. 19B. FACS analysis of PKH26-positive gut bacteria from mice orally administered PKH-26-labeled fecal exosomes. FIG. 19C. Confocal images of bacteria showing uptake of PKH-26-labeled fecal exosomes (red). FIG. 19D. BLASTN for the potential binding site of miR-375 with E. coli tryptophanase (tnaA) mRNA. FIG. 19E. mRNA levels of the tnaA gene in gut bacteria derived from HFD mice treated with PBS or GDNP. FIG. 19F. 2D LC-MS/MS analysis of unmetabolized tryptophan levels excreted into the feces of PBS- or GDNP-treated HFD-fed mice. FIG. 19G. Quantification of the indole levels in the feces (left panel) and plasma (right panel) obtained from lean and HFD-fed mice that were treated with PBS or GDNP. FIG. 19H. Fold change in tnaA gene expression in fecal bacteria (left), and indole estimation in the fecal supernatants (middle) and plasma (right) from RCD mice treated with PBS, Nano or Nano-miR375 nanoparticles. FIG. 19I. qPCR analysis of miR-375 levels in human fecal exosomes and plasma exosomes derived from healthy, obese and T2D individuals. FIG. 19J. Quantification of indole levels in the feces and plasma. FIG. 19K. Quantification of plasma cholesterol and triglyceride levels. FIG. 19L. Scatter plot depicting the linear correlation between cholesterol and miR-375 levels, and triglycerides and miR-375 levels. FIG. 19M. Principle component analysis (PCoA) of miR-375 and indole levels in obese, T2D and healthy human fecal exosomes. One-way ANOVA with the Bonferroni correction for multiple comparisons and Student t (one tailed) test were used to calculate statistical significance. (p value *<0.05; **<0.01; ***<0.001). One-way ANOVA with the Bonferroni correction for multiple comparisons and Student t (one tailed) test were used to calculate statistical significance (p value *<0.05; **<0.01; ***<0.001).

FIGS. 20A and 20B: miR-375 regulates tryptophan metabolism and indole production. FIG. 20A. A 2D LC-MS analysis of fecal supernatants of HFD-fed mice treated with PBS or GDNP for 1 month or 6 months. FIG. 20B. Linear correlation of indole vs cholesterol and triglycerides in healthy, obese, and T2D individuals. Pearson correlation coefficient test.

FIGS. 20A-20J: GDNP prevent the development of HFD-induced glucose intolerance, insulin resistance, inflammation, and decrease in lifespan. FIG. 20A. Body weights at various time points of diet administration (RCD or HFD). Statistical significance was calculated between PBS- and GDNP-treated HFD-fed mice. FIG. 20B. Images of the white adipose tissue (WAT) and liver in lean and PBS- or GDNP-treated HFD-fed mice. Fat deposition shown by red arrows. Liver weight after 12 months of PBS/GDNP treatment. FIG. 20C. Quantification of levels of circulating insulin (left panel) and glucose-induced insulin (right panel) in lean and PBS- or GDNP-treated HFD-fed mice. FIG. 20D. Glucose tolerance test (GTT) and insulin tolerance test (ITT) of lean and HFD-fed mice treated with PBS or GDNP at 12 months. One-way ANOVA with Bonferroni post hoc test was used for statistical significance. FIG. 20E. Quantification of plasma dextran FITC fluorescence in lean and HFD-fed mice treated with PBS and GDNP to determine the gut permeability. FIG. 20F. H & E staining of small intestine tissues from lean, and PBS- and GDNP-treated HFD-fed mice. FIG. 20G. Quantification of plasma levels of anti-inflammatory (IL-10) and pro-inflammatory (IL-1β, IL-6 and TNF-α) cytokines in lean and PBS- or GDNP-treated HFD-fed mice. FIG. 20H. Cytokine array for skin tissue obtained from lean and PBS- or GDNP-treated HFD-fed mice. Levels of pro-inflammatory cytokines are labeled with red circles. FIG. 20I. Representative images of the phenotypic changes induced by 12 months of HFD feeding. Note the changes in skin/fur color (red circle) and hair loss in PBS-treated HFD mice (n=5/group). FIG. 20J. Percentage survival during HFD feeding and treatment with GDNP vs PBS, compared to control lean animals. One-way ANOVA with the Bonferroni correction for multiple comparisons and nonparametric t (one tailed) tests were used to calculate statistical significance. (p value *<0.05; <0.01; ***<0.001).

FIGS. 22A-22L: miR-375 improves insulin sensitivity and glucose homeostasis and prevents dyslipidemia. FIG. 22A. Graphical representation of the experiment, which consisted of adoptive transfer of CD63+A33+ fecal exosomes (H-Exo) from HFD mouse (HFD fed 12 months) plus nanoparticles containing miR-375. Nanoparticles generated using the total lipid from GDNP. FIG. 22B. Live imaging of mice orally administered PKH26 labeled nanoparticles containing miR-375. FIG. 22C. Imaging of the liver, small and large intestines indicating the presence of labeled nanoparticles after 6 hours of oral administration. FIG. 22D. PKH26 labeled nanoparticle uptake by hepatocytes (albumin-positive cells) or kupffer (F4/80-positive) cells. FIG. 22E. Representative images of cellular uptake of PKH26-labeled nanoparticles by hepatocytes (albumin-positive cells). PKH26-labeled particles are indicated by pink arrows. FIG. 22F. 3D image of PKH26-labeled nanoparticles in hepatocytes. FIG. 22G. Confocal imaging to detect of AhR (FITC) and biotinylated miR-375 or scrambled microRNA in liver tissues derived from mice orally administered nanoparticles. FIG. 22H. GTT and ITT for C57BL/6 mice that received the fecal exosomes (H-Exo) along with nanoparticles contained miR-375 or scrambled RNA for 14 days while the mice were fed a HFD. Statistical to comparisons were made between H-Exo vs Nano-miR375; cause H-Exo responsible for insulin resistance and miR-375 preventing the development of insulin resistance. Nanoparticles contained scramble RNA (Nano-scramble); nanoparticles only (Nano); and nanoparticles contained miR-375 (Nano-miR375). FIG. 22I. Cholesterol and triglyceride levels in plasma derived from HFD mice treated with either PBS or nanoparticles (above mentioned) for 14 days. FIG. 22J. Insulin signaling array of mouse hepatocytes cultured with fecal exosomes (H-Exo) along with nanoparticles (contained scramble & nano-miR-375) showing alterations in genes involved in insulin signaling. Green-boxed genes promote insulin activity, and red-boxed genes inhibit insulin activity. FIG. 22K. Western blot depicting Foxa2, AhR, and IRS-1 and 2 expression in hepatocytes treated with fecal exosomes (H-Exo) along with nanoparticles (contained scramble & nano-miR-375). The ratio to β-actin shown below. FIG. 22L. Effect of fecal exosomes (H-Exo) on glucose uptake by hepatocytes. One-way ANOVA with a Bonferroni correction for multiple comparisons test was used to calculate statistical significance. (p value *<0.05; **<0.01; ***<0.001; ****<0. 0001).

### DETAILED DESCRIPTION

Obesity is a complex and chronic disease that affects more than a third of the world's population Changes in lifestyle, and particularly increased consumption of unhealthy diets, are thought to be major causes of the current epidemics of obesity and type 2 diabetics (T2D). This form of diabetes is characterized by insulin resistance. Given that among the numerous factors from a diet or dietary supplements that could contribute to modulate insulin signaling, identifying specific diet-derived factor(s) that contributes to modulate insulin signaling is challenging.

A high-fat diet contributes to obesity and insulin resistance, and diet manipulation is the basis of prevention and treatment of obesity and diabetes. The molecular mechanisms that mediate the diet-based prevention of insulin resistance, however, remain to be identified. Here, we report that treatment with orally administered ginger-derived nanoparticles (GDNP) can prevent insulin resistance by restoring homeostasis in gut epithelial Foxa2 and AhR mediated signaling in mice fed a high-fat diet (HFD). Mechanistically, HFD-feeding inhibited the expression of Foxa2, the GDNPs increased the expression of Foxa2 and protected against Akt-1 mediated inactivation of Foxa2. Furthermore, GDNP increased the expression of VAMP7 and miR-375 in intestinal epithelial cells in a Foxa2-dependent manner. In turn, miR-375 inhibited the expression of AhR, and VAMP7 sorted miR-375 from intestinal epithelial cells into exosomes following treatment with GDNP. Exosomal miR-375 then interacted with the gut Escherichia coli tryptophanase (tnaA) gene, inhibiting the production of the AhR ligand indole, and was also taken up by hepatocytes, leading to inhibition of hepatic AhR expression. Collectively, addition of GDNP into drinking water prevents insulin resistance in HFD mice. Interestingly, oral administration of GDNP also extended the lifespan of the mice and inhibited skin inflammation. Our findings that GDNPs can prevent HFD-induced obesity and insulin resistance will be critical for the development of therapeutic interventions for obesity.

A number of diet-derived factors regulate the aryl hydrocarbon receptor (AhR) mediated signaling pathway which has been shown to regulate insulin response. Mice that express a low-affinity AhR allele were less susceptible to obesity after exposure to a HFD and exhibited differences in fat mass, liver physiology and hepatocyte gene expression compared to mice with high-affinity AhR. Serum AhR ligand levels were increased in T2D patient samples and correlated with measures of insulin resistance. However, the molecular mediators and mechanisms governing the association between diet, AhR and insulin pathway signaling in general are still elusive.

While AhR-mediated pathways promote the development of obesity, studies on mice and humans have suggested that chronic consumption of a HFD causes inactivation of the transcription factor Foxa2. Inactivation of a transcription factor, Foxa2, leads to the development of T2D. Moreover, a previous study found that Foxa2 expression is an important determinant in preventing disease onset and decreasing its severity. Foxa2 is essential for glucose and lipid homeostasis. Tissue-specific deletion of Foxa2 in pancreatic β cells in mice led to increased adiposity under a high-fat diet conditions and decreased adipocyte glucose uptake and glycolysis. Whether diet-derived factors regulate the activity of Foxa2 is not known. Furthermore, little is known about the relationship between AhR- and Foxa2-mediated pathways in terms of regulating the insulin response.

Exosomes are bilayer membrane vesicles released by almost every mammalian cell type for intercellular communication. Human intestinal epithelial cells secrete exosomes bearing accessory molecules that may be involved in antigen presentation, maintenance of intestinal tract immune balance, and have been implicated in regulating the homeostasis of gut microbiota and lymphocyte homing to the gut. However, determining the roles that exosomes might play in liver/gut axis communication requires a better understanding of their composition, in particular, whether diet alters the PC and/or PE content of intestinal epithelial exosomes and hence their biological functions in terms of insulin response has not been studied.

One protein of interest in the regulating insulin response is the aryl hydrocarbon receptor (AhR), a ligand-activated transcription factor that integrates dietary and metabolic cues to control the complex transcriptional program. Indeed, AhR overexpression leads to insulin resistance. Conversely, germline AhR null mice have enhanced insulin sensitivity and improved glucose tolerance. Moreover, mice that express a low-affinity AHR allele are less susceptible to obesity after exposure to a HF diet and exhibit differences in fat mass, liver physiology and hepatocyte gene expression compared to mice with high-affinity AhR. However, the molecular mediators and mechanisms governing the diet-mediated association between AhR, PC lipid, and insulin pathway signaling in hepatocytes are largely unknown.

Disclosed herein are studies that reveal that a HF diet dramatically changes the lipid profile of intestinal epithelial exosomes from predominantly PE to PC, which results in inhibition of the insulin response via binding of exosomal PC to AHR expressed in hepatocytes and suppression of genes essential for activation of the insulin pathway. These results revealed a mechanism by which diet shapes the exosome lipid profile of intestinal epithelial cells to regulate liver/gut axis communication.

Also disclosed herein are experiments wherein GDNP was employed as a proof-of-concept to study the GDNP effect on gut epithelial Foxa2 and AhR mediated signaling in mice fed a HFD. HFD-fed mice given GDNPs via gavage showed improved glucose tolerance and insulin response. Moreover, it was found that a HFD inhibited Foxa2 expression, and gut epithelial cell uptake of GDNP prevented HFD-mediated inhibition of Foxa2 expression and signaling. The findings presented herein also indicated that Foxa2-regulated transcriptional pathways modulated insulin signaling by inhibition of both hepatic and intestinal epithelial AhR expression in mice via Foxa2 mediated induction of miR375 and VAMP7.

### I. Definitions

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. Mention of techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

Following long-standing patent law convention, the terms “a”, “an”, and “the” refer to “one or more” when used in this application, including the claims.

The term “about”, as used herein to refer to a measurable value such as an amount of weight, time, dose (e.g., therapeutic dose), etc., is meant to encompass in some embodiments variations of ±20%, in some embodiments ±10%, in some embodiments ±5%, in some embodiments ±1%, in some embodiments ±0.1%, in some embodiments ±0.5%, and in some embodiments ±0.01% from the specified amount, as such variations are appropriate to perform the disclosed methods.

As used herein, the term “and/or” when used in the context of a list of entities, refers to the entities being present singly or in any possible combination or subcombination. Thus, for example, the phrase “A, B, C, and/or D” includes A, B, C, and D individually, but also includes any and all combinations and subcombinations of A, B, C, and D.

The term “comprising”, which is synonymous with “including” “containing”, or “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting of” excludes any element, step, or ingredient not specifically recited. For example, when the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of the related disclosure or claim to the specified materials and/or steps, plus those that do not materially affect the basic and novel characteristic(s) of the disclosed and/or claimed subject matter. For example, a therapeutic method of the presently disclosed subject matter can “consist essentially of” one or more enumerated steps as set forth herein, which means that the one or more enumerated steps produce most or substantially all of the therapeutic benefit intended to be produced by the claimed method. It is noted, however, that additional steps can be encompassed within the scope of such a therapeutic method, provided that the additional steps do not substantially contribute to the therapeutic benefit for which the therapeutic method is intended.

With respect to the terms “comprising”, “consisting essentially of”, and “consisting of”, where one of these three terms is used herein, the presently disclosed and claimed subject matter can include the use of either of the other two terms. Similarly, it is also understood that in some embodiments the methods of the presently disclosed subject matter comprise the steps that are disclosed herein, in some embodiments the methods of the presently disclosed subject matter consist essentially of the steps that are disclosed, and in some embodiments the methods of the presently disclosed subject matter consist of the steps that are disclosed herein.

As used herein, the term “active agent” refers to any bioactive molecule for which delivery to a subject, such as but not limited to delivery via a liposome, exosome, or plant-derived nanoparticle might be desired. Exemplary active agents include therapeutic agents, diagnostic agents, and detectable agents. More particularly, exemplary active agents can include bioactive small molecules and bioactive nucleic acids, including but not limited to miRNAs.

### II. Exemplary Methods

In some embodiments, the presently disclosed subject matter relates to methods for increasing insulin sensitivity in a subject in need thereof. In some embodiments, the methods comprise, consist esstentially of, or consist of administering to the subject an effective amount of a composition comprising a lipid bilayer, wherein the lipid bilayer is low in total phosphatidylcholine (PC) or has been treated to reduce total PC. As used herein, the phrase “low in total phosphatidylcholine (PC) or has been treated to reduce total PC” refers to a composition comprising a lipid bilayer wherein the PC content of the lipid bilayer has a total PC content in relation to total lipids or is treated with a process to reduce the PC content of the lipid bilayer of in some embodiments less than about 20% PC, in some embodiments less than about 15% PC, in some embodiments less than about 12% PC, in some embodiments less than about 11% PC, in some embodiments less than about 10% PC, in some embodiments less than about 9% PC, in some embodiments less than about 8% PC, in some embodiments less than about 7% PC, in some embodiments less than about 6% PC, to in some embodiments less than about 5% PC, in some embodiments less than about 4% PC, in some embodiments less than about 3% PC, in some embodiments less than about 2% PC, and in some embodiments less than about 1% PC. In some embodiments, the lipid bilayer of the composition has a lysophosphatidylcholine (LPC) contect of less than about 15%, in some embodiments less than about 14%, in some embodiments less than about 13%, in some embodiments less than about 12%, in some embodiments less than about 11%, in some embodiments less than about 10%, in some embodiments less than about 5%, and in some embodiments less than about 3% in relation to total lipids. In some embodiments, the lipid bilayer of the composition has an ether-phosphatidylcholine (ePC) content of about 10% or less.

As described herein, exosomes, including but not limited to intestinal exosomes, as well as ginger-derived nanoparticles (GDNPs) are examples of compositions with lipid bilayers that have PC, LPC, and/or ePC contents that fall within these ranges. Thus, in some embodiments the methods of the presently disclosed subject matter employ exosomes including but not limited to intestinal exosomes, as well as ginger-derived nanoparticles (GDNPs) as components of the compositions that can be administered to subjects. In some embodiments, an exosome employed in the presently disclosed methods is an exosome that has been purified from a biological sample that itself was isolated from the subject such that in some embodiments the presently disclosed methods relate to isolating and purifying “self” exosomes from a subject and administering the same back to the subject. Methods for isolating and purifying exosomes from biological samples are known in the art and are described herein. Methods for isolating and purifying GDNPs are also described herein as well as in U.S. Pat. No. 9,717,733 and U.S. Patent Application Publication Nos. 2018/0140654, 2018/0291433, 2018/0362974, 2019/0380962, 2020/0063208, 2020/0188311, and 2020/0206297, each of which is incorporated herein by reference in its entirety.

Also disclosed herein are methods for diagnosing insulin sensitivity and/or a metabolic-related disorder of the liver in a subject. In some embodiments, the methods comprise, consist essentially of, or consist of assaying total phosphatidylcholine (PC) of intestinal exosomes isolated from the subject. In some embodiments, a determination that the total PC content of the intestinal exosomes isolated from the subject is elevated relative to intestinal exosomes isolated from a normal subject is indicative of insulin sensitivity and/or a metabolic-related disorder of the liver in the subject. Thus, in some embodiments the presently disclosed subject matter provides methods for identifying subjects with insulin sensitivity and/or a metabolic-related disorder of the liver that comprise, consist essentially of, or consist of assaying total phosphatidylcholine (PC) of intestinal exosomes isolated from the subject. In some embodiments, a total PC content of the intestinal exosomes isolated from the subject exceeds about 14% lysophosphatidylcholine (LPC) and/or about 10% ether-phosphatidylcholine (ePC), and/or about 10% PC as compared to total lipids is indicative of the subject having insulin sensitivity and/or a metabolic-related disorder of the liver, and identifying a subject with intestinal exosomes that have LPC and/or ePC and/or PC contents in excess of these values is considered to identify a subject with insulin sensitivity and/or a metabolic-related disorder of the liver.

Also disclosed herein are methods for inhibiting development of insulin resistance, optionally insulin resistance associated with diabetes, in subjects in need thereof. In some embodiments, the methods comprise, consist essentially of, or consist of administering to the subject a composition comprising, consisting essentially of, or consisting of exosomes, including but not limited to intestinal exosomes, and/or ginger-derived nanoparticles (GDNPs) in an amount and via a route sufficient to inhibit development of insulin resistance in the subject. In some embodiments, the composition comprising, consisting essentially of, or consisting of exosomes, including but not limited to intestinal exosomes, and/or GDNPs is administered to the subject orally. In some embodiments, the development of insulin resistance is incident to a high fat diet consumed by the subject.

Also disclosed herein are methods for preferentially targeting hepatocytes in a subject for delivery of an active agent. In some embodiments, the methods comprise, consist essentially of, or consist of administering to a subject a composition comprising a lipid bilayer, optionally a nanoparticle, with a low total PC content and/or enhanced total phosphatidylethanolamine (PE) content, wherein the composition preferentially targets the subject's hepatocytes. As disclosed herein, compositions comprising lipid bilayers wherein the total PE content of the lipid bilayer comprises PE of at least 50%, ether-phosphoethanolamine (ePE) of at least 30%, or both preferentially target to hepatocytes. It is possible to load lipid bilayer compositions with active agents without affecting the lipid content of the lipid bilayer, and thus exosomes, including but not limited to liver exosomes and/or GDNPs that have or are modified to have low total PC content and/or enhanced total PE content can be employed as delivery vehicles to deliver active agents to hepatocytes. In some embodiments, the exosome, optionally the intestinal exosome, and/or the GDNP has been treated to reduce the total PC content to less than about 35%, 30%, 25%, 20% or lower and/or to enhance the total PE content to greater than about 35%, 40%, 45% or 50% or higher and/or to enhance the ePE content to at least about 30%, 35%, 40% or higher. Methods for loading lipid bilayers with active agents are described herein, as well as in U.S. Pat. No. 9,717,733 and U.S. Patent Application Publication Nos. 2018/0140654, 2018/0362974, 2019/0365658, 2019/0380962, and 2020/0188311, each of which is incorporated by reference in its entirety.

Similarly, the presently disclosed subject matter also provides methods for preferentially targeting liver macrophages and/or monocytes in subjects. In some embodiments, the methods comprise, consist essentially of, or consist of administering to a subject a composition comprising a lipid bilayer, optionally a nanoparticle, further optionally an exosome including but not limited to an intestinal exosome, and in some embodiments a GDNP with a high total PC content and/or a reduced total PE content, wherein the composition preferentially targets the subject's liver macrophages and/or monocytes. As set forth herein, compositions comprising, consisting essentially of, or consisting of lipid bilayers with total PE content of less than 35% (e.g., less than 35%, less than 30%, less than 25%, or less than 20%), ether-phosphoethanolamine (ePE) of less than 30% (e.g., less than 30%, less than 25%, or less than 20%), or both target liver macrophages and/or monocytes. As such, compositions comprising lipid bilayers with PE and/or ePE contents within these parameters can be employed to deliver active agents to liver macrophages and/or monocytes. In some embodiments, the compositions comprise nanoparticles such as but not limited to GDNPs and/or exosomes, optionally intestinal exosomes, and further optionally intestinal exosomes that have been isolated from the subject. In some embodiments, the composition comprises intestinal exosomes, wherein the intenstinal exosome have a total PC content greater than about 35% and/or a total PE content of less than about 35%.

Also provided are methods for restoring homeostasis in gut epithelium in subjects in need thereof. In some embodiments, the methods comprise administering to the subject a ginger-derived nanoparticle (GDNP) in an amount and via a route sufficient to restore homeostasis in gut epithelium in the subject.

Also provided are methods for enhancing expression of a Foxa2 gene product in a cell, the method comprising contacting the cell with a ginger-derived nanoparticle (GDNP) in an amount sufficient to enhance expression of the Foxa2 gene product in the cell. Also provided are methods for inhibiting Akt-1-mediated inactivation of a Foxa2 biological activity in a subject, the method comprising administering to the subject a ginger-derived nanoparticle (GDNP) in an amount and via a route sufficient to inhibit Akt-1-mediated inactivation of a Foxa2 biological activity in the subject.

Also provided are methods for increasing expression of VAMP7, miR-375, or both in an epithelial cell, optionally an epithelial cell present in a subject, the method comprising contacting the epithelial cells with a ginger-derived nanoparticle (GDNP) in an amount sufficient to increase expression of VAMP7, miR-375, or both in the epithelial cell.

Also provided are methods for enhancing sorting of miR-375 from intestinal epithelial cells to exosomes, the method comprising contacting the intestinal epithelial cells with a ginger-derived nanoparticle (GDNP) in an amount sufficient to enhance sorting of miR-375 from the intestinal epithelial cells to exosomes. In some embodiments, the intestinal epithelial cells are present in a subject.

Also provided are methods for inhibiting hepatic AhR expression in a subject, the method comprising administering to the subject a ginger-derived nanoparticle (GDNP) in an amount and via a route sufficient to enhance sorting of miR-375 from intestinal epithelial cells to exosomes in the subject, whereby the exosomes are taken up by hepatocytes in the subject in an amount sufficient to inhibit hepatic applicants hereby reserve expression in the subject.

Also provided are methods for inhibiting development of obesity in a subject in need thereof, the method comprising administering to the subject a ginger-derived nanoparticle (GDNP) in an amount and via a route sufficient to inhibit development of obesity in the subject.

In some embodiments of any or all of the presently disclosed methods, the subject is a mammalian subject, optionally a human subject. In some embodiments, the cell is a mammalian cell, optionally a human cell.

### III. Exemplary Compositions

As such, the presently disclosed subject matter also relates in some embodiments to compositions for use in the presently disclosed methods, including compositions for diagnosing, preventing, and/or treating a disease, disorder, and/or condition; and/or for identifying subjects with a disease, disorder, and/or condition; and/or for preferentially targeting hepatocytes, liver macrophages and/or monocytes in subjects; and/or for restoring homeostasis in gut epithelium in subjects in need thereof; and/or for enhancing expression of Foxa2 gene products in cells; and/or for inhibiting Akt-1-mediated inactivation of Foxa2 biological activities in cells and/or subjects; and/or for increasing expression of VAMP7, miR-375, or both in epithelial cells; and/or for enhancing sorting of miR-375 from intestinal epithelial cells to exosomes; and/or for inhibiting hepatic AhR expression in subjects; and/or for inhibiting development of obesity in subjects in need thereof. In some embodiments, the compositions comprise lipid bilayer-containing components, which in some embodiments are nanoparticles and/or exosomes, optionally intestinal exosomes, further optionally intestinal exosomes isolated from subjects, and/or ginger-derived nanoparticles (GDNPs).

Methods for isolating and modifying nanoparticles including but not limited to GDNPs and exosomes, including but not limited to loading and/or coating the nanoparticles and/or exosomes with active agents, can be found, for example, in U.S. Patent Application Publication Nos. 2012/0315324, 2014/0308212, 2017/0035700, 2018/0140654, and 2018/0362974, in PCT International Patent Application Publication No. WO 2019/104242, and in U.S. Pat. No. 9,717,733, each of which is incorporated herein by reference in its entirety.

III.A. Formulations

The compositions of the presently disclosed subject matter can be administered in any formulation or route that would be expected to deliver the compositions to the subjects and/or target sites present therein.

The compositions of the presently disclosed subject matter comprise in some embodiments a composition that includes a carrier, particularly a pharmaceutically acceptable carrier, such as but not limited to a carrier pharmaceutically acceptable in humans. Any suitable pharmaceutical formulation can be used to prepare the compositions for administration to a subject. For example, suitable formulations can include aqueous and non-aqueous sterile injection solutions that can contain anti-oxidants, buffers, bacteriostatics, bactericidal antibiotics, and solutes that render the formulation isotonic with the bodily fluids of the intended recipient.

It should be understood that in addition to the ingredients particularly mentioned above the formulations of the presently disclosed subject matter can include other agents conventional in the art with regard to the type of formulation in question. For example, sterile pyrogen-free aqueous and non-aqueous solutions can be used.

The therapeutic regimens and compositions of the presently disclosed subject matter can be used with additional adjuvants or biological response modifiers including, but not limited to, cytokines and other immunomodulating compounds.

III.B. Routes of Administration

By way of example and not limitation, suitable methods for administering a composition in accordance with the methods of the presently disclosed subject matter include, but are not limited to, systemic administration, parenteral administration (including intravascular, intramuscular, and/or intraarterial administration), oral delivery, buccal delivery, rectal delivery, subcutaneous administration, intraperitoneal administration, inhalation, intratracheal installation, surgical implantation, transdermal delivery, local injection, intranasal delivery, and hyper-velocity injection/bombardment. Where applicable, continuous infusion can enhance drug accumulation at a target site (see e.g., U.S. Pat. No. 6,180,082, which is incorporated herein by reference in its entirety). In some embodiments, a composition comprising a nanoparticle and/or an exosome is administered orally.

Thus, exemplary routes of administration include parenteral, enteral, intravenous, intraarterial, intracardiac, intrapericardial, intraosseal, intracutaneous, subcutaneous, intradermal, subdermal, transdermal, intrathecal, intramuscular, intraperitoneal, intrasternal, parenchymatous, oral, sublingual, buccal, inhalational, and intranasal. The selection of a particular route of administration can be made based at least in part on the nature of the formulation and the ultimate target site where the compositions of the presently disclosed subject matter are desired to act. In some embodiments, the method of administration encompasses features for regionalized delivery or accumulation of the compositions at the site in need of treatment. In some embodiments, the compositions are delivered directly into the site to be treated.

III.C. Dose

An effective dose of a composition of the presently disclosed subject matter is administered to a subject in need thereof. An “effective amount” or a “therapeutic amount” is an amount of a composition sufficient to produce a measurable response. Exemplary responses include biologically or clinically relevant responses in subjects such as but not limited to an increase in insulin sensitivity, a inhibition of or reduction in obesity, an improvement in a metabolic-related disorder or a symptom thereof, accumulation of a lipid bilayer in a hepatocyte, macrophage, and/or monocyte, an improve in or retoration of gut epithelium homeostasis, an enhancement of Foxa2 gene expression, an inhibition of an Akt-1-mediated inactivation of a Foxa2 biological activity, an increase in expression of VAMP7, miR-375, or both in epithelial cells, etc.). Actual dosage levels of the compositions of the presently disclosed subject matter can be varied so as to administer an amount of the composition that is effective to achieve the desired response for a particular subject. The selected dosage level will depend upon the activity of the composition, the route of administration, combination with other drugs or treatments, the severity of the disease, disorder, and/or condition being treated, and the condition and prior medical history of the subject being treated. However, it is within the skill of the art to start doses of the compositions of the presently disclosed subject matter at levels lower than required to achieve the desired therapeutic effect and to gradually increase the dosage until the desired effect is achieved. The potency of a composition can vary, and therefore an “effective amount” can vary. However, using the methods described herein, one skilled in the art can readily assess the potency and efficacy of a composition of the presently disclosed subject matter and adjust the regimen accordingly.

As such, after review of the instant disclosure, one of ordinary skill in the art can tailor the dosages to an individual subject, taking into account the particular formulation, method of administration to be used with the composition, and particular disease, disorder, and/or condition treated or biologically relevant outcome desired. Further calculations of dose can consider subject height and weight, severity and stage of symptoms, and the presence of additional deleterious physical conditions. Such adjustments or variations, as well as evaluation of when and how to make such adjustments or variations, are well known to those of ordinary skill in the art.

### EXAMPLES

The presently disclosed subject matter will be now be described more fully hereinafter with reference to the accompanying EXAMPLES, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

### Materials and Methods for Examples 1-8

Mice. 8- to 12- week-old male C57BL/6 mice were purchased from the Jackson Laboratory (Bar Harbor, Me.) and maintained on a 12-h/12-h light/dark cycle in a pathogen-free animal facility at the University of Louisville. Mice were fed a regular chow diet or a high-fat diet during the study. AhR knockout mice were purchased from Taconic Biosciences (Rensselaer, N.Y.). Germ-free mice were purchased from the National Gnotobiotic Rodent Resource Center (University of North Carolina, Chapel Hill, N.C.) and maintained in flexible film isolators (Taconic Biosciences) at the Clean Mouse Facility of the University of Louisville (Louisville, KY.). Animal care was performed following the Institute for Laboratory Animal Research (ILAR) guidelines, and all animal experiments were conducted in accordance with protocols approved by the University of Louisville Institutional Animal Care and Use Committee (Louisville, Ky.).

Cells. Murine hepatocytes (FL83B) and human hepatocytes (HepG2) (obtained from the American Type Culture Collection, ATCC®, Manassas, Va.) were grown in tissue culture plates with F12K medium (Thermo Fisher Scientific,) supplemented with 10% heat-inactivated fetal bovine serum (FBS), 100 U ml-1 penicillin, and 100 mg ml-1 streptomycin at 37° C. in a 5% CO₂ atmosphere. Human monocytes (U937) were grown in RPMI 1640 medium (Thermo Fisher Scientific, Waltham, Mass.) supplemented with 10% FBS. For fecal exosomes (with or without PKH26 labeling) treatment for FL83B and HepG2 cells, 2×10⁴ cells were seeded into six well plates. After achieving 50-60% confluence, fecal exosomes (numbers indicated in Figures) were added and incubated for 16 hours at 37° C. in a 5% CO₂ atmosphere. Cells were washed with PBS and processed for imaging, RNA isolation, and protein extraction. The C57BL/6 murine colon carcinoma MC-38 cell line and human embryonic kidney 293 cells (ATCC®) were grown at 37° C. in 5% CO₂ in Dulbecco's modified Eagle's medium (DMEM; Thermo Fisher Scientific) supplemented with 10% heat-inactivated FBS, 100 U ml⁻¹ penicillin and 100 μg ml⁻¹ streptomycin.

Human subjects. The study involved five healthy volunteers between the ages of 25 to 45 years (all males) and five Type 2 diabetes (T2D) patients. No healthy volunteers had a history of chronic gastrointestinal disease. All volunteers were recruited from the University of Louisville Hospital, Louisville, Ky., USA. Type 2 diabetes was diagnosed according to the American Diabetes Association diagnostic criteria (American Diabetes Association 2012). All clinical fecal samples were collected from patients in the outpatient endocrinology clinic. All participants were educated regarding their participation and signed a written consent form. Approval for the study was granted by the University of Louisville Research Ethics committee.

Generation of colon epithelial MC38 cells expressing GFP in mouse colon. 8 to 12-week-old C57BL/6 male mice (n=5 per group) were anaesthetized with a mixture of ketamine and xylazine by intraperitoneal injection and 0.5×10⁶ of green fluorescent protein (GFP) labeled-MC38 colon cancer cells or PBS were administered via endoscopy-guided colonic submucosal injection. After six weeks, mice were euthanatized and livers, mesenchymal lymph nodes (MLNs), and spleens were collected for histological evaluation.

Isolation and purification of feces exosomes. Feces pellets were re-suspended in PBS and minced manually. Differential centrifugation was deployed to isolate the feces exosomes. Fecal suspension was centrifuged at 1000×g for 10 minutes, 2,000×g for 20 minutes, and 4,000×g for 30 minutes to remove larger and junk particles. The supernatant was centrifuged at 8,000×g for 1 hour to remove the micro-particles. Finally, the suspension was centrifuged at 100,000×g for 2 hours. Pellets were suspended in PBS. The exosomes were further purified by sucrose gradient (8, 30, 45, and 60% sucrose in 20 mM Tris-Cl, pH 7.2) centrifugation. An aliquot of the purified exosomes was fixed in 2% paraformaldehyde for transmission electron microscopy (EM) using a conventional procedure and observed using an FEI Tecnai F20 sent to EM facility equipped at the University of Alabama (Birmingham, Ala., USA). The EM was done with the following settings: 80 kV at a magnification of 15,000 and defocus of 100 and 500 nm.

Nanoparticle tracking analysis. Purified exosome samples were analyzed for particle concentration and size distribution using the nanoparticle tracking analysis method provided by the Malvern NanoSight NS300 (Malvern Instruments Ltd, Malvern, United Kingdom). The assays were performed in accordance with the manufacturer's instructions. Briefly, for the NanoSight, three independent replicates of diluted exosome preparations in PBS were injected at a constant rate into the tracking chamber using the provided syringe pump. The specimens were tracked at room temperature for 60 seconds. Shutter and gain were manually adjusted for optimal detection and were kept at optimized settings for all samples. The data were captured and analyzed with NTA Build 127 software (version 2.2; Malvern Instruments Ltd).

For labelled or stained exosomes, the sample was first run without any fluorescent channel and then the sample was run into a specific (PE) fluorescent channel. Percent positivity was calculated as fluorescent positive exosomes/total exosomes x 100.

Immuno-isolation of exosomes. A standard method for immune-isolation was followed. Briefly, antibodies for immuno-isolation (mouse monoclonal anti-human CD63; NBP2-32830 0.1 mg; Novus Biologicals LLC, Centennial, Colo.) and normal mouse polyclonal IgG (Cat. No. 12-371; Millipore, Burlington, Mass.) at a ratio of 1 μg of antibody per 100 μL of beads were coupled to Pierce Protein A Magnetic Beads (DYNABEADS®) by overnight incubation at 4° C. Beads were then washed three times with 500 μL of PBS 0.001% Tween, and re-suspended in 500 μL of the same buffer, to which exosomes (2×10¹⁰) were added followed by overnight incubation at 4° C. with rotation. Bead-bound exosomes were collected and washed three times in 500 μL PBS-Tween. Exosomes were eluted with high salt buffer and again washed and centrifuged at 100,000×g for 1 hour at 4° C. in a TLA 110 rotor (Optima TL100 Centrifuge, Beckman Coulter, Indianapolis, Ind.).

Liquid chromatography-mass spectrometry (LC-MS) analysis. LC-MS was carried out. Acquired high-resolution data was analyzed using Proteome Discoverer v1.4.1.114 (Thermo Fisher Scientific) with Matrix Science Mascot v2.5.1 and SequestHT searches and the 2/17/2017 version of the mouse proteome from UniprotKB (Proteome ID UP000000955). Scaffold Q+S (ProteomeSoftware) used the Peptide and Protein Prophet algorithms to model and calculate the data by the false discovery rate. Proteins were grouped to satisfy the parsimony principle. The proteins were clustered based on differential expression, and heat maps representing differentially regulated proteins by feces exosomes were generated using R software.

Quantitative reverse transcription PCR (qPCR) analysis mRNA expression. Total RNA was isolated from tissue and cells using a miRNeasy mini kit (Qiagen Inc. Valencia, Calif.). For analysis of AHR, IRS-2, IGF1R, IGF2, LDLR, PTPRF, and JUN mRNA expression, 1 μg of total RNA was reverse transcribed using SuperScript III reverse transcriptase (Invitrogen) and quantitation was performed using primers (Eurofins) with QuantiTect SYBR Green PCR (Qiagen). GAPDH was used for normalization. The primer sequences are listed in Table 1. qPCR was run using the BioRad CFX96 qPCR System with each reaction run in triplicate. Analysis and fold change were determined using the comparative threshold cycle (Ct) method. The change in miRNA or mRNA expression was calculated as fold change.

TABLE 1
List of Mouse Primers Employed

| | Target | |
|---|---|---|
| | name | Primer sequence 5′-3′ |
| 1 | mAHR-F | GCAATAGCTACTCCACTTCAG; SEQ ID NO: 1 |
| | mAHR-R | GGTGTGAAGTCTAGCTTGTG; SEQ ID NO: 2 |
| 2 | mIRS2-F | GTCCAGGCACTGGAGCTTT; SEQ ID NO: 3 |
| | mIRS2- | GCTGGTAGCGCTTCACTCTT; SEQ ID NO: 4 |
| 3 | mIGF1R-F | TGACATCCGCAACGACTATCA; SEQ ID NO: 5 |
| | mIGF1R-R | CCAGTGCGTAGTTGTAGAAGAGT; SEQ ID NO: 6 |
| 4 | mIGF2-F | GTGCTGCATCGCTGCTTAC; SEQ ID NO: 7 |
| | mIGF2-R | CGGTCCGAACAGACAAACTG; SEQ ID NO: 8 |
| 5 | mLDLR-F | TCAGACGAACAAGGCTGTCC; SEQ ID NO: 9 |
| | mLDLR-R | CCATCTAGGCAATCTCGGTCTC; SEQ ID NO: 10 |
| 6 | mPTPRF-F | TGCTCTCGTGATGCTTGGTTT; SEQ ID NO: 11 |
| | mPTPRF-R | ATCCACGTAATTCGAGGCTTG; SEQ ID NO: 12 |
| 7 | mJUN-F | TTCCTCCAGTCCGAGAGCG; SEQ ID NO: 13 |
| | mJUN-R | TGAGAAGGTCCGAGTTCTTGG; SEQ ID NO: 14 |

miRNA PCR array. miRNA expression profiling for exosomes was performed using the Qiagen miScript miRNA PCR Array Mouse miRBase Profiler (Cat# 331223) using an Applied Biosystems ViiA 7 Real-Time PCR System. Normalization to endogenous control genes included SNORD61, SNORD68, SNORD72, SNORD95m and RNU6 to correct for potential RNA input or RT efficiency biases. miRNA data generated from exosomes were comparatively analyzed by the online free data analysis software available at the Qiagen website. Quantile normalization and subsequent data processing were performed using software R. Scatter plots representing differentially regulated genes were generated using software R.

Immuno-staining of exosomes. Immuno-staining was carried out. Exosomes suspended in PBS were incubated with 5% BSA for 1 hour at room temperature (RT) and washed three times with PBS and primary antibodies added at (1:1000) or directly conjugated antibodies and incubated at 4° C. overnight. The mixture was washed three times with PBS and fluorescent secondary antibody (1:2000 dilution) was added and incubated at RT for 1 hour. The mixture was washed again with PBS and the pellet was dissolved in PBS. Finally, the pellet was re-suspended into PBS and passed through a 200 nM syringe filter to disaggregate the exosomes.

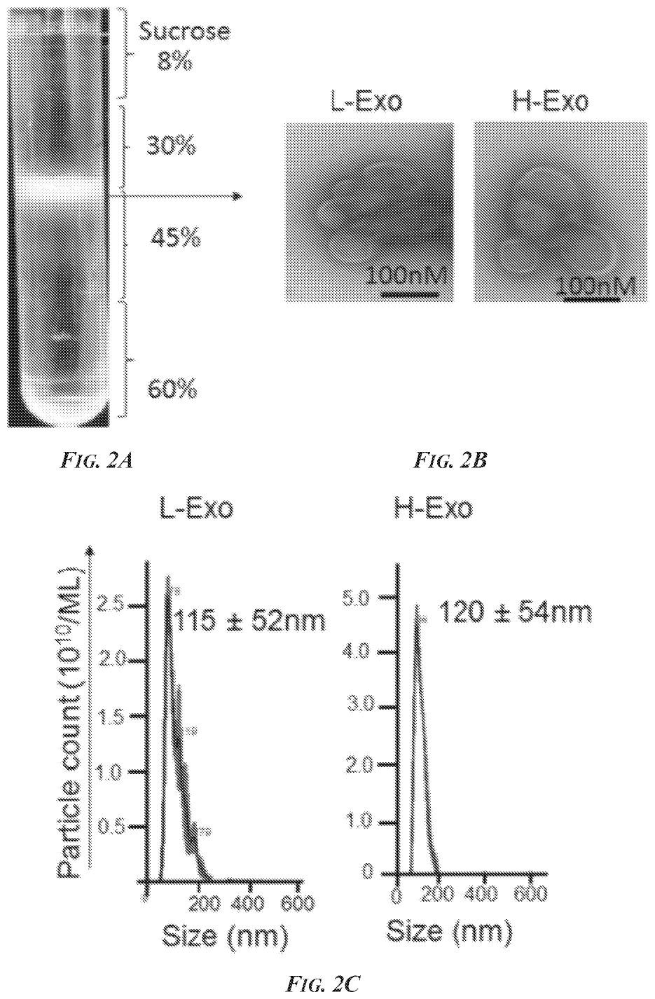

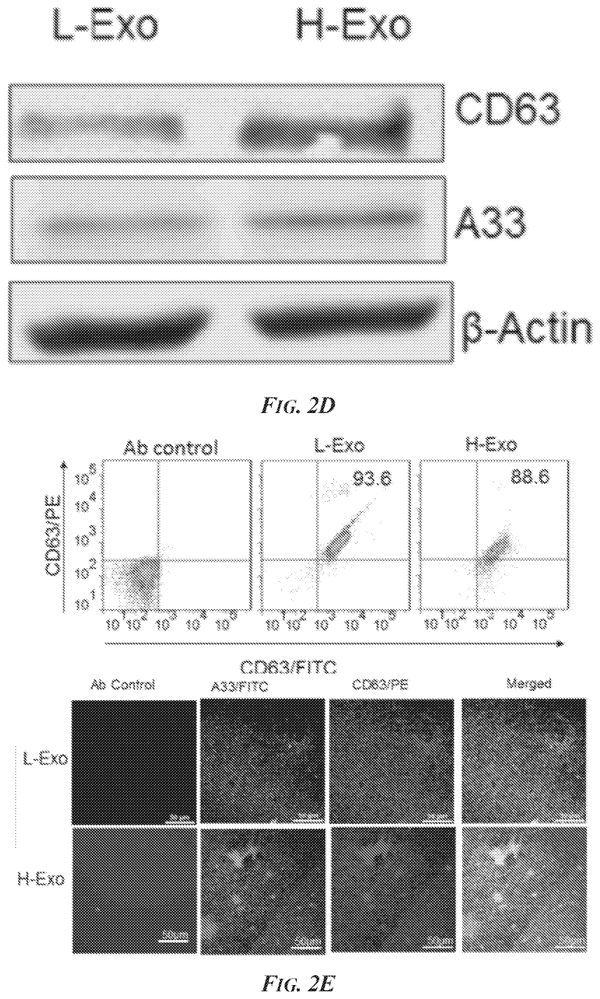

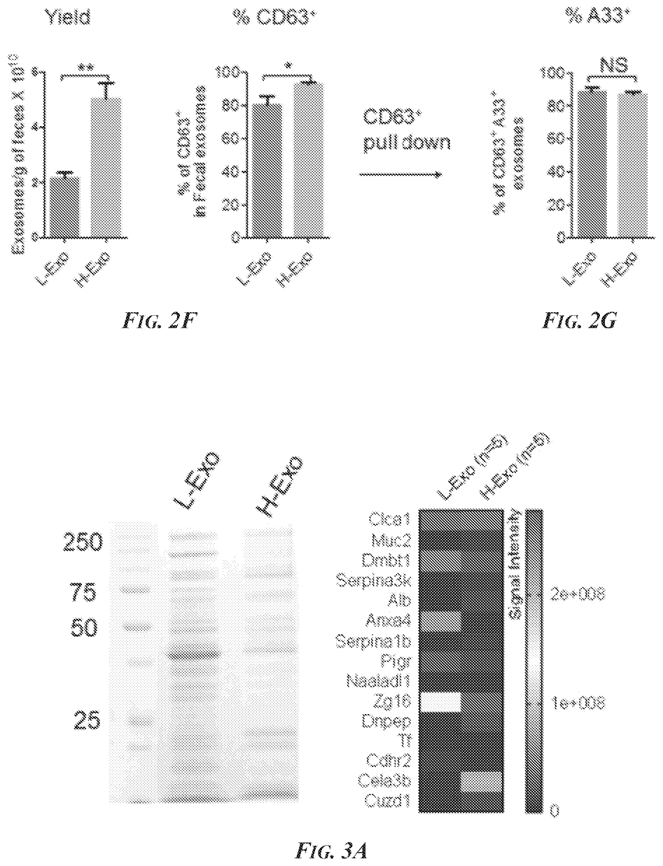

Lipid extraction from feces exosomes. Total lipids were extracted from a sucrose gradient band (FIG. 2A) of processed fecal exosomes. Briefly, 1.9 ml of a 2:1 (v/v) MeOH:CHCl₃ mixture was added to 0.5 ml (2×10¹²) of exosomes in PBS. 0.625 ml of CHCl₃ and water (1:1) were added sequentially and vortexed thoroughly. The aqueous and organic phases were separated by centrifugation at 850×g for 10 minutes at 22° C. in glass tubes. The organic phase was collected using a glass pipette. The organic phase was aspirated and dispensed into fresh glass tubes. The organic phase was dried by heating under nitrogen (2 psi) and dried overnight under vacuum. Total lipids were determined using a phosphate assay.

Lipidomic analysis with MS. Lipids extracted from feces exosomes were submitted to the Lipidomics Research Center, Kansas State University (Manhattan, Kans.) for analysis using MS55. In brief, the lipid composition was determined using triple quadrupole MS (Applied Biosystems Q-TRAP, Applied Biosystems, Foster City, Calif.). The data are reported as the concentration (nmol mg-1 feces exosomes) and percentage of each lipid within the total signal for the molecular species determined after normalization of the signals to internal standards of the same lipid class.

HPLC analyses of phosphoethanolamine (PE) and phosphatidylcholine (PC). The lipids extracted from fecal exosomes were diluted with an equal volume of methanol and filtered through a 0.22 nM filter. 25 μl of lipids in methanol were injected for high-performance liquid chromatography (HPLC) analysis. The HPLC analysis was performed on an Agilent 1260 Infinity system equipped with an Agilent 300, SB-C8 column (4.6×250 mm, 5 μm), with the following parameters: mobile phase A: water with 0.1% formic acid; mobile phase B: 100% acetonitrile modified with 0.1% formic acid (v/v); gradient: 10% B in first 5 minutes, 10-95% B for 10 minutes, hold 95% B for 5 minutes, 95%-10% B for 5 minutes, with a 2 minute post run. Flow rate: 0.5 ml/min; temperature: 30° C. UV detection at 220 nm was used to monitor PE and PC. The standards for PE and PC were purchased from Avanti Polar Lipids (Alabaster, Ala.; Catalog Nos. 841118C-25 mg and 850458C-25 mg, respectively).

Transfections experiments. Mouse hepatocytes (FL83B) were transfected with 200 ng of construct pGL3B-PEMT-luc (kindly provided Dr. Jongsook Kim Kemper, Department of Molecular and Integrative Physiology, University of Illinois at Urbana, Ill.) and pBABE puro mouse IRS-2 (Cat. No. 11371; Addgene, Watertown, Mass.) were used. Transfections were performed using a kit from Invitrogen (Cat. No. L3000-015) in accordance with manufacturer's instructions.

Luciferase assay. pGL3B-PEMT-luc plasmid transfected FL83B cells were treated with 100 μl of fecal metabolites from either RCD or HFD mice for 16 hours at 37° C. in a CO₂ incubator. Luciferase activity was measured using dual luciferase system (Cat. No. E1910; Promega Corp., Madison, Wis.) as per manufacturer's instructions.

Glucose and Insulin tolerance tests (GTT and ITT). 2×10⁹/dose exosomes were orally administered to each mouse for 14 days. For glucose tolerance tests, after an overnight fast, baseline glucose levels were determined using a glucometer (Prodigy Diabetes Care, LLC, Charlotte, N.C.). Then, mice were given an intraperitoneal injection of glucose (dextrose) at a dose of 2 mg/g of body weight. Blood glucose levels were measured at 30, 60, 90, and 120 minutes after glucose injection. For insulin tolerance tests, mice were fasted for 4-6 hours and basal blood glucose levels were determined. Mice were then given an intraperitoneal injection of insulin (1.2 units per gram of body weight). Blood glucose levels were measured at 30, 60, and 90 minutes (unless otherwise indicated in Figures) after insulin injection.

Thin-layer chromatography (TLC) analysis. Total lipids from fecal exosomes were quantitatively analyzed and used for TLC analysis. Briefly, HPTLC-plates (silica gel 60 with a concentrating zone, 20 cm×10 cm; Merck) were used for the separation. After extracting samples of concentrated lipid from fecal exosomes, the lipids were separated on a plate that had been developed with chloroform/methanol/acetic acid (190:9:1, by vol). After drying in air, the plates were sprayed with a 10% copper sulfate and 8% phosphoric acid solution and then charred by heating at 120° C. for 5-10 minutes. The bands of lipid on the plates were imaged using an Odyssey Scanner (Licor Bioscience, Lincoln, Nebr.).

Generation of the GFP-MC38 cell line releasing green fluorescent protein (GFP) exosomes. A lentivirus preparation was made. Stable HEK293T cells expressing GFP were generated by transfecting the GFP expression plasmids (PalMGFP; kindly provided by Xandra O. Breakefield, Department of Neurology and Radiology, Massachusetts General Hospital, Harvard Medical School, Charlestown, Mass.). The plasmid was transfected with lentivirus packing vectors pCMVdelta8.2 and VSV-G using the Lipofectamine 3000 transfection kit (Invitrogen). Pseudovirus-containing culture medium was collected after 72 hours of transfection and the viral titer was estimated. PalMGFP expressing lentivirus were used to generate GFP-MC38 cells. MC38 (2×10⁵ ) cells were dispensed into a six-well plate along with an appropriate amount of viral stock in the medium. After selection by puromycin, the cells with the highest expression of GFP were sorted using a BD FACSAria III cell sorter (BD Biosciences, San Jose, Calif.) and used further. GFP expression was further confirmed by confocal fluorescence microscopy (Nikon, Melville, N.Y.).

For AhR knock out (AhRKO) cells, CRISPR/CAS9 (sc-419054) plasmid for mouse AhR was purchased from Santa Cruz Biotechnology Inc. (Dallas, Tex.). Pseudovirus and lentiviral particles were generated as above described herein.

Nanoparticle preparation. Total lipids from fecal exosomes were extracted with chloroform and dried under vacuum. 200 nM of lipid was suspended in 200-400 μl of 155 nM NaCl. After ultraviolet (UV) irradiation at 500 mJ/cm² in a Spectrolinker crosslinker (Spectronic Corp., Westbury, N.Y.) and a bath sonication (F S60 bath sonicator, Fisher Scientific) for 30 minutes, the nanoparticles were collected by centrifugation at 100,000×g for 1 hour at 4° C.

Labelling of nanoparticles and fecal exosomes. Nanoparticle or fecal exosomes were labeled with DIR or PKH26 Fluorescent Cell Linker Kits (Sigma) using the manufacturer's instructions. Nanoparticle or fecal exosomes were suspended in 250 μl of diluent C with 4 μl of DIR or PKH26 dye and subsequently incubated for 30 minutes at room temperature. After washing with PBS and centrifugation at 100,000×g for 1 hour at 4° C., the pellet was re-suspended in PBS and used in experiments.

Western blot analysis. The tissues were washed with ice cold PBS and homogenized. The cells were lysed in radioimmunoprecipitation assay (RIPA) lysis buffer with addition of protease inhibitor for 1 hour at 4° C. The crude lysates were centrifuged at 14,000×g for 15 minutes. Protein concentrations were determined using the BioRad Protein Assay Reagent. Samples were diluted in SDS sample buffer. Proteins were separated by 10% SDS-PAGE and transferred to nitrocellulose membranes (Bio-Rad). Individual protein was detected with specific antibodies and visualized by infrared fluorescent secondary antibodies (Table 2). The protein bands were visualized and analyzed on an Odyssey CLx Imager (LiCor Inc, Lincoln, Nebr.).

TABLE 2
List of Antibodies Employed

| Target | Source | Cat. No. | Application |
|---|---|---|---|
| AHR | Santa Cruz | sc133088 | Western blot |
| | Biotechnology | | |
| pAHR | ThermoScientific | PA5-38404 | Western blot |
| pIRS-2 | Abcam | Ab3690 | Western blot |
| CD63 | Biolegend | 143902 | Western blot/Flow |
| | | | cytometry/ |
| | | | immunofluorescence |
| | | | (IF) |
| CD63 | Novus | NBP2-32830 | Pull down |
| | Biologicals | | |
| A33 | Biorybt | orb15687 | Western blot/Flow |
| | | | cytometry |
| PEMT | ThermoScientific | PA5-42383 | Western blot |
| Albumin | Cell Signaling | 4929S | Western blot/IF |
| F4/80 | eBioscience | | Flow cytometry |
| F4/80 | eBioscience | | IF |
| TNF-α | ThermoScientific | 39-8321-60 | ELISA |
| TNF-α | R&D Systems | AF-410-SP | Neutralization |
| IL-6 | R&D Systems | MAB406 | ELISA |
| IL-6 | R&D Systems | MAB406 | Neutralization |
| CD14 | eBioscience | 12-0141-81 | Flow cytometry |
| GAPDH | Santa Cruz | sc47724 | Western blot |
| Histone | Santa Cruz | sc10807 | Western blot |
| β-Actin | Santa Cruz | sc47778 | Western blot |
| IgG | Santa Cruz | sc65662 | Pull down |

Flow cytometry. The livers of mice were perfused with perfusion buffer ((Ca²⁺-Mg²⁺-free HBSS containing 0.5 mM EGTA, 10 mM HEPES and 4.2 mM NaHCO₃ supplemented with Type I collagenase (0.05%) and trypsin inhibitor (50 μg/ml; pH 7.2)) and then harvested into complete medium. Cells isolated from liver tissue were fixed with 2% paraformaldehyde (PFA) and stained with albumin and F4/80 primary antibodies for 40 minutes at 4° C. After three washes with PBS, cells were stained with ALEXA FLUOR® 488 or PE conjugated secondary antibodies for 1 hour at RT. Stained liver cells (monocytes and hepatocytes) treated with PKH26+ nanoparticle or fecal exosomes were acquired using a BD FACSCanto flow cytometer (BD Biosciences, San Jose, Calif.) and analyzed using FlowJo software (Tree Star Inc., Ashland, Oreg.).

Confocal microscopy. For frozen sections, periodate-lysine-paraformaldehyde (PLP) fixed tissues were dehydrated with 30% sucrose in PBS overnight at 4° C. and embedded into optimal cutting temperature (OCT) compound. Tissue was subsequently cut into ultrathin slices (5 μm) using a microtome. The tissue sections were blocked with 5% bovine serum albumin (BSA) in PBS. Primary antibodies (1:800) were added and incubated at 4° C. overnight. Sections were washed three times followed by secondary antibodies conjugated to a fluorescent dye (at 1:2000 dilution). Nuclei were stained with 4′, 6-diamidino-2-phenylindole dihydrochloride (DAPI). For in vitro cultured cells, 2×10⁵ cells were grown on coverslips in six well plates and co-cultured with PKH26 labeled feces exosomes for 16 hours at 37° C. in a CO₂ incubator. Cells were washed with PBS and fixed with 2% PFA. Nuclei were stained with DAPI. Tissues and cells were visualized via confocal laser scanning microscopy (Nikon, Melville, N.Y.).

Histological analysis. For hematoxylin and eosin (H&E) staining, tissues were fixed with buffered 10% formalin solution (SF93-20; Fisher Scientific, Fair Lawn, N.J.) overnight at 4° C. Dehydration was achieved by sequential immersion in a graded ethanol series of 70%, 80%, 95%, and 100% ethanol for 40 minutes each. Tissues were embedded in paraffin and subsequently cut into ultrathin slices (5 μm) using a microtome. Tissue sections were deparaffinized in xylene (Fisher), rehydrated in decreasing concentrations of ethanol in PBS, stained with H&E, and the slides were scanned with an Aperio ScanScope (Leica Biosystems, Wetzlar, Germany).

Glucose uptake assay. Glucose uptake was performed using the Glucose Uptake-Glo™ Assay from Promega (J1341) in accordance with the manufacturer's instructions. Briefly, 2×10⁴ cells were seeded into 96 well tissue culture plate in complete medium. When cells achieved 50-60% confluency, fecal exosomes (1×10⁶) and PBS as control were added and incubated for 16 hours at 37° C. in a CO₂ incubator. Cells were treated with 1 nM of insulin for an additional 1 hour. Medium was removed and cells were washed twice with PBS. 50 μof 2-deoxyglucose (DG, 1 mM per well) was added and incubated for 1 hour at room temperature (RT). 25 μl of stop buffer added and mixed briefly, then 25 μl of neutralization buffer added and shaken briefly. 100 μl of 2DG6P detection reagent was added and the mixture shaken for 3 hours at RT. Luminescence was recorded with 135 gain efficiency using a SYNERGY H1 (BioTek Instruments, Inc., Winooski, Vt.) luminometer.

For glucose uptake testing, hepatocytes were cultured with supernatant from monocytes (U937) pre-cultured with fecal exosomes (2×10⁶ L-Exo or 1×10⁵, 5×10⁵, 1×10⁶, 2×10⁶, or 5×10⁶ H-Exo. U937 cells (2×10⁵) were seeded into six well plates and when the cells reached 50-60% confluence, fecal exosomes (2×10⁶) were added and incubated for 16 hours at 37° C. in a CO₂ incubator. Culture supernatants were harvested and centrifuged at 100,000×g to remove fecal exosomes. These supernatants (0.5 ml) were further used in hepatocyte (cultured with 0.5×10⁶ fecal exosomes) cultures to determine the which cytokines were induced by fecal exosome treatment of macrophages.

Cytokine production in peripheral blood. To investigate effects of fecal exosomes on the regulation of cytokine production in peripheral blood, peripheral blood was collected from mice orally administered exosomes for 14 days and plasma was extracted. Cytokines were assayed with a PROTEOME PROFILER™ Mouse XL Cytokine Array Kit (Catalog No. ARY028; R&D Systems, Minneapolis, Minn.) per the manufacturer's instructions. Quantification of the spot intensity in the arrays was conducted with background subtraction using HLImage++ (Western Vision Software, Salt Lake City, Utah).

Macrophage depletion. For macrophage depletion, CLODROSOME® brand macrophage depletion reagent (Encapsula Nano Sciences, Brentwood, Tenn.) was used in accordance with the manufacturer's instructions. In brief, a single intravenous injection (100 μl) of CLODROSOME® brand macrophage depletion reagent was given to each mouse and 72 hours later macrophage depletion was confirmed by whole blood staining of F4/80 and analysis by flow cytometry.

Enzyme-linked immunosorbent assay (ELISA). Tumor necrosis factor (TNF)-α and interleukin (IL)-6 levels in plasma were quantified using an ELISA method. ELISA reagents were purchased from eBioscience and assays were performed in accordance with the manufacturer's instructions. Briefly, a microtiter plate was coated with anti-mouse IL-6 and TNF-α antibody at 1:200 overnight at 4° C. Excess binding sites were blocked with 100 μl/well of blocking solution (PBS containing 0.5% BSA) at room temperature for 1 hour. After washing three times with PBS containing 0.05% Tween 20, sera collected from mice were diluted 2-fold, added in a final volume of 50 μl to the plate wells and incubated for 1 hour at 37° C. After 3 washes with PBS, the plate was incubated with 100 μl of HRP-conjugated anti-mouse antibody (Pierce) diluted 1:50,000 in blocking solution for 1 hour at RT. After the final 3 washes with PBS, the reaction was developed for 15 min, blocked with H2SO4 and optical densities were recorded at 450 nm using a microtiter plate reader (BioTek Synergy HT).

Neutralization of TNF-α and IL-6. To neutralize TNF-α and IL-6 in conditioned media (CM) before adding the CM to the hepatocytes (FL83B) cultures, the harvested CM was pre-incubated at 37° C. for 1 hour with a rat anti-TNF-α antibody (R&D system), a rat anti-IL-6 antibody (R&D System), or with a mixture of both antibodies. The neutralizing dose50 (ND₅₀) for the anti-TNF-α antibody was 0.2 μg/ml. For rat anti-IL-6, 1.0 μg ml-1 antibody was used based on the ND₅₀ provided by R&D System. Normal rat IgG at the same concentration as the anti-TNF-α and anti-IL-6 antibodies was used as a control.

Affymetrix array. Total mRNA was extracted isolated from tissues using Qaigen total RNA isolation kit (Cat. No. 74104). 100 ng of RNA for each sample submitted to Invitrogen/ThermoFisher Scientific Affymetrix facility, Santa Carla, Calif. Transcriptome Analysis Console (TAC) 4.0 from ThermoFisher Scientific was used to analyze the data.

Surface plasmon resonance (SPR). SPR experiments were conducted on an OPENSPR™ (Nicoya Lifesciences, Ontario, Canada). Experiments were performed on a LIP-1 sensor and NTA sensor (Nicoya Lifesciences). Tests were run at a flow rate of 20 μl/min using HBS running buffer (20 mM HEPES, 150 mM NaCl, pH 7.4). First, the LIP-1 sensor chip was cleaned with octyl β-D-glucopyranoside (40 mM) and CHAPS (20 mM). Liposomes (1 mg/ml) were injected on the sensor chip for 10 min until stable resonance was obtained. After immobilization of nanoparticles, the surface was blocked with BSA (3%) in running buffer. After a stable signal was obtained, recombinant AhR protein (Cat. No. OPCD01209; Aviva Systems Biology Corp., San Diego, Calif.) was run over the immobilized liposomes. A negative control test was also performed by injecting protein onto a blank sensor chip to check for non-specific binding. After 10 minutes, the nanoparticles binding protein were eluted using NaOH (200 μM). For NTA sensor (protein), AhR protein was injected (0.5 mg/ml) for 10 minutes until stable resonance was obtained. After immobilization of protein, nanoparticles were run over the immobilized protein. The sensograms were analyzed using TRACEDRAWER™ kinetic analysis software (Ridgeview Instruments AB, Vänge, Sweden)

Direct binding of PC lipid with AhR. 5 nM PC or PE lipid was coated onto 96 well plates in 200 μl of 1× coating buffer (Cat. No. 00-0044-59; eBioscience) for overnight at 4° C. Wells were washed three times with 1× wash buffer (PBS with 0.05% TWEEN® 20) and blocked with ELISPOT buffer (Cat. No. 00-4952-54; eBioscience) for an hour at RT. After washing the wells, recombinant AhR protein (0.5 mg/ml) in 100 μl A of diluent buffer (Cat. No. 00-4202-55; eBioscience) was added and incubated for two hours at RT. After appropriate washing, anti-AhR antibody (1:1000) in 100 μl of diluent buffer was added and incubated for 1 hour at RT and subsequently detected with fluorochrome conjugated secondary (anti-mouse) antibody and plates was scanned using an Odyssey Scanner (Licor Bioscience, Lincoln, Nebr.).

Insulin signaling Array. 1 μg of total RNA from FL83B cells treated PBS, L-Exo, or H-Exo was reverse transcribed using Superscript III reverse transcriptase (Invitrogen). Insulin signaling array (PAMM030ZE) from Qiagen was performed on Applied Biosystems ViiA 7 Real-Time PCR System in accordance with manufacture's instructions.

Lipids analysis in peripheral blood. Peripheral blood samples from mice were collected into non-heparinized capillary tubes coated with 4% sodium citrate. The levels of cholesterol and triglycerides were determined by a PICCOLO® lipid panel plus (Abaxis, Inc, Union City, Calif.).

Statistical analysis. Statistical significance was determined using the Student's one tailed t-test or one-way analysis of variance (ANOVA) with post-test for multiple comparisons or two-way ANOVA as appropriate. GraphPad Prism 5.0 and 7.0 (GraphPad Software, San Diego, Calif.) were used for data analysis. Results are presented as mean±standard deviation (SD). p values <0.05 were considered statistically significant. NS—not significant. * p<0.05; ** p<0.01; *** p<0.001.

### Example 1

### A High-fat Diet Altered the Composition of Intestinal Epithelial CD63+A33+ Exosomes

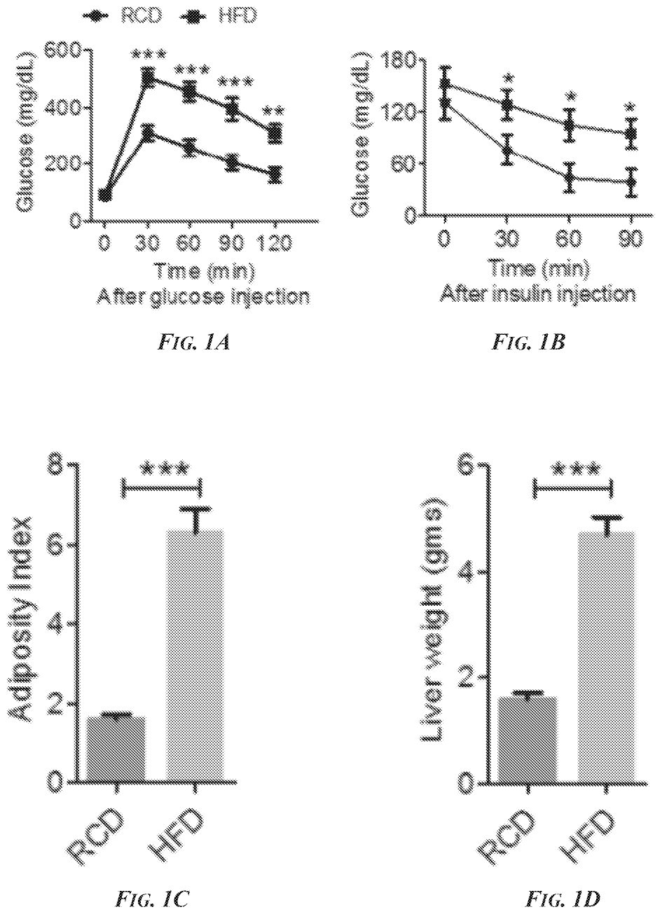

To study the effect of high-fat diet (HFD) on intestinal epithelial-released exosomes, a 12-month HFD-induced obesity mouse model was employed. Fecal exosomes were isolated at 2, 6, and 12 months on regular chow diet (RCD) or HFD. HFD mice developed glucose intolerance (FIG. 1A) and insulin resistance (FIG. 1B) compared to mice fed a regular chow diet (RCD) for 12 months. The HFD mice were obese and had an increased adiposity index (FIG. 1C), as well as fatty liver and steatosis (FIG. 1D) as determined by Oil O red staining of liver sections.

Exosomes were isolated from the feces of a group of 12-month HFD mice (H-Exo) and age- and sex-matched lean RCD mice (L-Exo) by differential centrifugation. Sucrose purified exosomes (FIG. 2A) from lean and HFD mice were characterized by transmission electron microscopy (FIG. 2B). Exosome size was estimated by nanoparticle tracking analysis, and the size ranges of L-Exo and H-Exo were 115±52 nM and 120±54 nm (FIG. 2C), respectively. Both L-Exo and H-Exo exosomes were positive for CD63 (exosome marker) and A33 (intestinal epithelial cell marker) as assessed by western blot (FIG. 2D) and by flow cytometry analysis and confocal microscopy (FIG. 2E). Significantly higher numbers of exosomes were isolated from HFD mice (˜5×10¹⁰ nanoparticles/g feces) than from lean RCD mice (˜2×10¹⁰ nanoparticles/g feces; FIG. 2F). However, no significant difference in the number of CD63+A33+ double-positive exosomes was found in HFD mice compared to lean RCD mice (FIG. 2G), suggesting that the number of exosomes released from intestinal epithelial cells is not affected by a HFD.

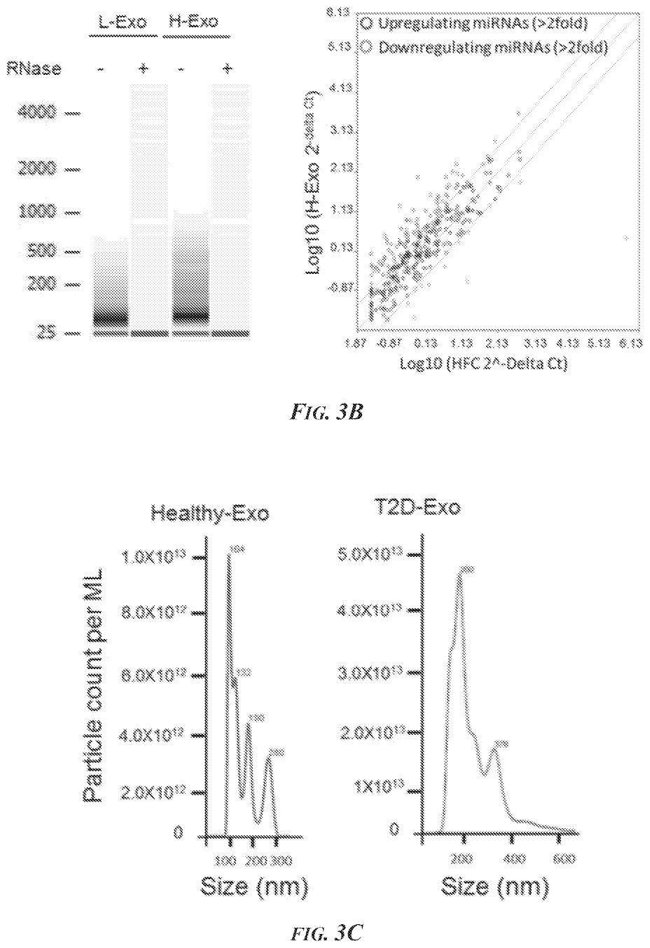

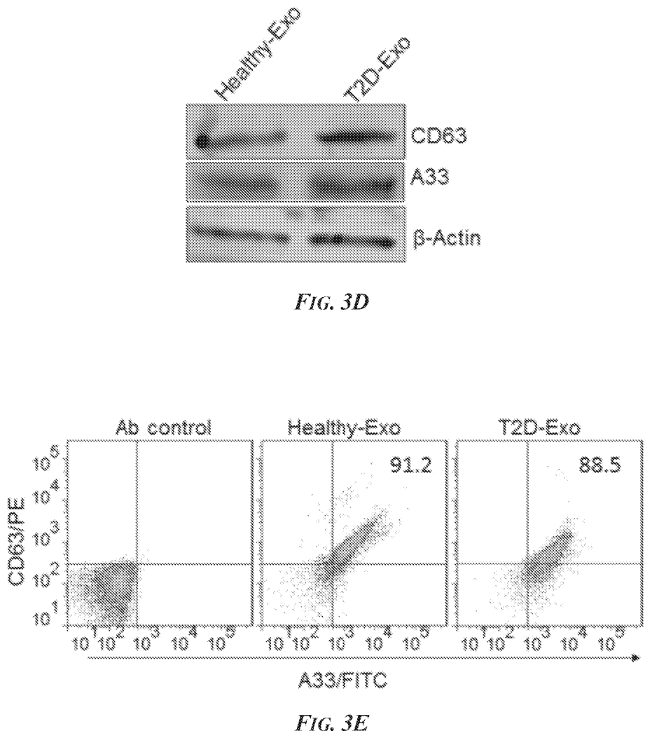

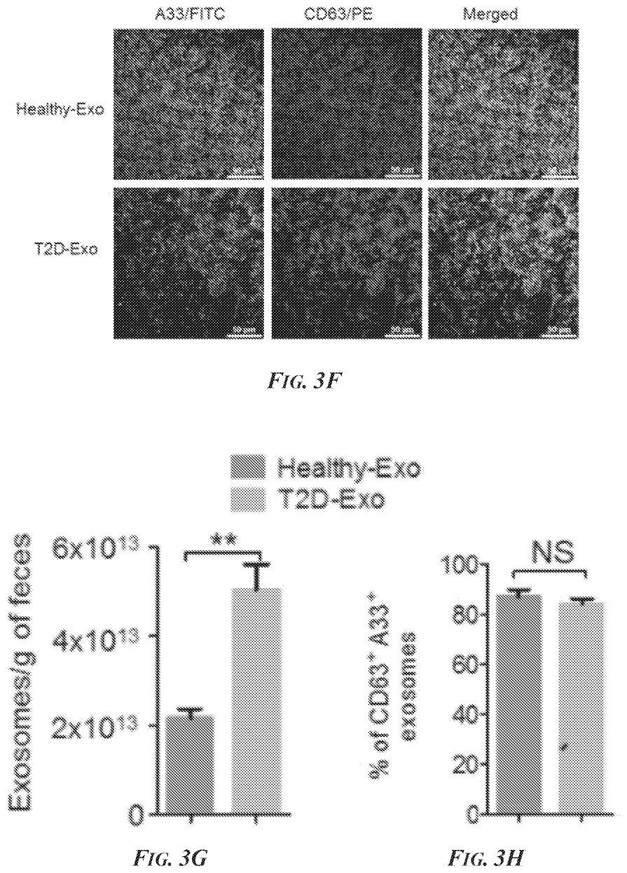

Whether HFD affected the composition of CD63+A33+ exosomes was next assessed. While changes in protein (FIG. 3A) and miRNA expression were noted (FIG. 3B) the exosomal lipid profile was the most dramatically affected by HFD (see Table 3).Quadrupole mass spectrometry (MS) analysis of total lipids revealed that L-Exo (collected after 6 months of feeding on RCD) was enriched in PE (53%) and ether-phosphoethanolamine (ePE; 37%), whereas H-Exo collected after 6 months of HFD feeding was enriched in lysophosphatidylcholine (LPC; 14%), ether-phosphatidylcholine (ePC; 10%), and PC, 10%. Furthermore, 12 months after HFD feeding, the percentages of PC lipids in H-Exo dramatically increased, as LPC increased to 51%, and PC increased to 34% (ePC decreased to 3%). Notably, the L-Exo lipid composition was not significantly altered after 12 months vs. 6 months of RCD feeding.

TABLE 3
Exosomal Lipid Profiles at 6 and 12 Months on HFD

| | L-Exo at | L-Exo at | H-Exo at | H-Exo at |
|---|---|---|---|---|
| | 6 months (%) | 12 months (%) | 6 months (%) | 12 months (%) |
| PE | 53 | 55 | 9 | 2 |
| PC | 0 | 0 | 10 | 34 |
| LPC | 1 | 0 | 14 | 51 |
| ePC | 0 | 1 | 10 | 3 |
| SAM | 2 | n.d | 14 | n.d. |
| PA | 1 | 1 | 6 | 2 |
| PG | 1 | 1 | 10 | 5 |
| ePS | 1 | 1 | 3 | 0 |
| PS | 3 | 2 | 9 | 1 |
| PI | 1 | 1 | 8 | 1 |
| ePE | 37 | 39 | 7 | 1 |

The dynamic changes in exosomal PE and PC levels were determined using HPLC analysis of CD63+A33+ exosomes from RCD and HFD mice at 3, 6, and 12 months of feeding with their respective diet. In agreement with the MS analysis, HPLC analysis also showed that H-Exo contained increased levels of PC compared to lean RCD mice after 6 and 12 months of HFD (˜40 μM and 80 μM; p<0.001 at each time point).

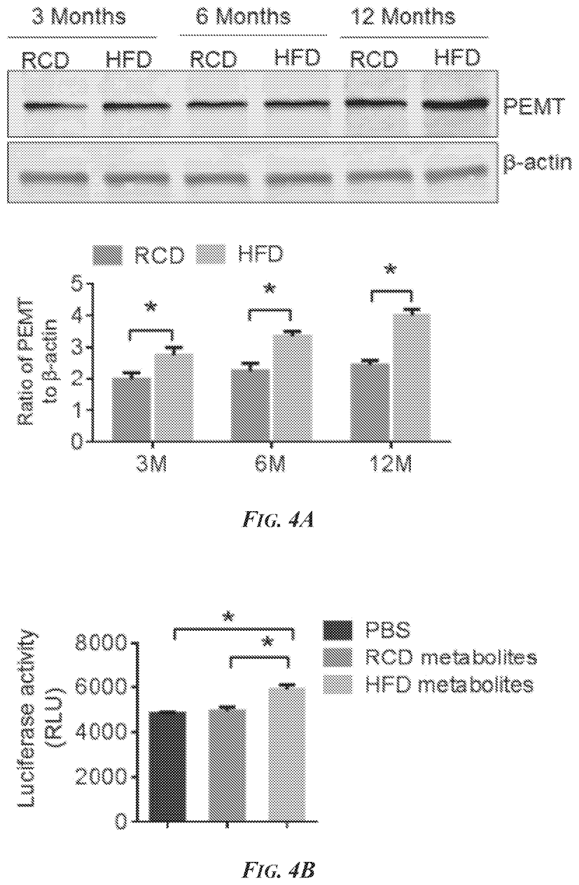

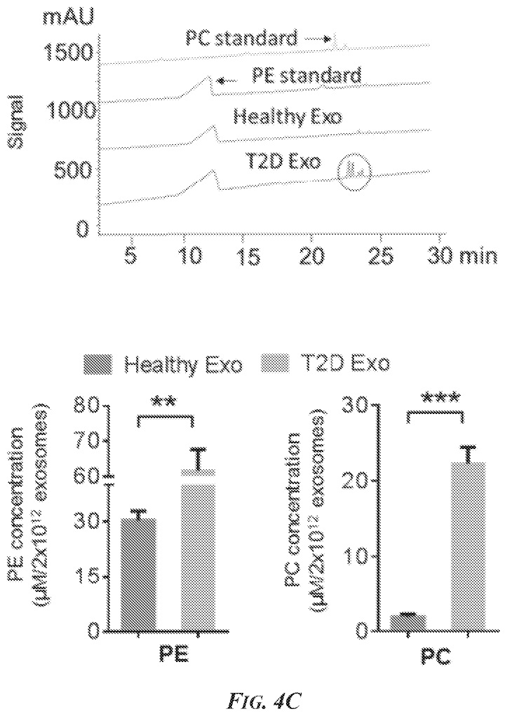

Whether phosphatidylethanolamine N-methyl transferase (PEMT), the transferase enzyme that converts PE to PC, was increased in the intestinal tissue of mice fed a HFD vs RCD lean controls was determined. Western blot analysis indicated that over the time course of 3, 6, and 12 months of feeding on their respective diets, the levels of PEMT increased in intestinal tissue of HFD mice compared to lean mice (FIG. 4A). Moreover, hepatocytes (FL83B) treated with the fecal metabolites from HFD mice showed increased expression of the luciferase gene driven by the PEMT promoter (FIG. 4B), suggesting that the expression of the gene encoding PEMT was modulated by a HFD. Collectively, these results suggested that HFD shifted the lipid composition of intestinal epithelial exosomes from predominantly PE to PC, potentially via an increase in levels of PEMT.

### Example 2

The Composition of Intestinal Epithelial CD63+A33+ Exosomes was Altered in Insulin Resistant Type II Diabetes Patients

To determine whether the findings in obese mice applied also to insulin resistant type II diabetes in humans, exosomes isolated from stool samples of insulin-resistant type II diabetic (T2D) patients and healthy subjects were characterized. The size ranges of the xxosomes derived from healthy subjects (Healthy-Exo) and diabetic-derived exosomes (T2D-Exo) estimated by nanoparticle tracking analysis were 104±81 nM and 190±156 nm, respectively (FIG. 3C). As in mice, the human-derived exosomes were positive for CD63 and A33 (FIGS. 3D-3F). Significantly higher numbers of total exosomes, as measured by the nanoparticle weight, were found in the stool samples of diabetic patients (˜5×10¹³ nanoparticles/g feces) than in those from healthy subjects (˜1×10¹³; FIG. 3G). However, no significant difference in the number of CD63+A33+ double-positive exosomes was found in diabetic patients compared to healthy subjects (FIG. 3H).

Next, PE and PC from stool exosome samples from healthy and T2D patients were quantitatively analyzed with quadrupole MS. Similar to the results from mice, T2D patient exosomes also carried elevated levels of PC (10%) compared to healthy individuals (˜0%; see also Table 4), whereas no change of LPC (1%). In agreement with the MS analysis, HPLC quantitative analysis also showed elevated levels of PC (˜40 μM) in T2D Exo (FIG. 4C), whereas in healthy-Exo, PC was undetectable.

TABLE 4
Exosomal Lipid Profiles of Healthy and T2D Subjects

| | Healthy-Exo (%) | T2D-Exo (%) |
|---|---|---|
| PE | 22 | 46 |
| PC | 0 | 10 |
| LPC | 1 | 1 |
| ePC | 2 | 1 |
| SAM | 23 | 1 |
| PA | 3 | 1 |
| PG | 9 | 2 |
| ePS | 2 | 2 |
| PS | 18 | 7 |
| PI | 8 | 4 |
| ePE | 12 | 25 |

### Example 3

### H-Exo and T2D-Exo Contributed to the Development of Insulin Resistance and Glucose Intolerance

Whether the altered lipid profile of H-Exo played a role in the response to glucose and insulin sensitivity was tested. To assess the in vivo effect of H-Exo, lean mice were given either CD63+A33+ L-Exo or H-Exo (2×10⁹/dose in 200 μPBS, 3, 5, 10, or 14 doses) by gavage every day for 14 days while being fed a HFD. H-Exo (at any dose) had no effect on the body weight of mice over the 14-day treatment period but 10 doses of H-Exo resulted in a dose-dependent impairment in glucose tolerance and insulin sensitivity (p<0.05 at 30 and 60 minutes after insulin injection). Fourteen doses of H-Exo resulted in further impairments in glucose tolerance and insulin sensitivity, as mice showed insulin resistance at all time points after insulin injection following 14 doses of H-Exo (p<0.05 at 30, 60, and 90 minutes after insulin injection). Based on this result, 14 doses of H-Exo were given for all mouse experiments throughout the study. Overall these results showed that H-Exo caused insulin resistance and glucose intolerance in mice.

Next, it was determined whether CD63+A33+ T2D-Exo from the stool of T2D human patients elicited glucose intolerance and insulin resistance in C57BL/6 SPF mice. Mice were treated with human CD63+A33+ T2D-Exo for 14 days while being fed a HFD. T2D-Exo had no effect on the body weight of mice over the 14-day treatment period. While the effects were not as pronounced as those observed after H-Exo treatment, GTT and ITT results suggested that mice that received T2D-Exo developed glucose intolerance and insulin resistance.

To determine whether the deleterious effect of H-Exo on the insulin response was dependent on the gut microbiome, aged-matched SPF and germ-free C57BL/6 male mice were orally administered H-Exo or L-Exo for 14 days while being fed a HFD. Germ-free mice were maintained in a germ-free environment during the experiments. After two weeks of H-Exo/L-Exo administration, glucose and insulin tolerance tests (GTT and ITT) were performed. Surprisingly, H-Exo treatment led both SPF and germ-free mice to develop glucose intolerance and reduced the response to insulin. These results suggest that H-Exo has a negative impact on the insulin response that is independent of the gut microbiome.

Since dynamic changes in PC levels in H-Exo were observed, the role of exosomal lipids in regulating insulin sensitivity was examined. To this end, nanoparticles were generated from total lipids of both L-Exo and H-Exo (2×10⁹ exosomes). To generate H-Exo-derived nanoparticles with depleted PC, total lipids from exosomes were extracted with chloroform and separated via thin layer chromatography (TLC) and PC was then depleted from H-Exo (H-Exo^{nano} PC-) while a fixed amount of PC was added to L-Exo (L-Exo^{nano} PC+). The band containing PC was identified based on standard PC migration in TLC and then removed. These lipid nanoparticles were orally administered to HFD-fed mice for 14 days. Again, the nanoparticles had no effect on the body weight of mice over the 14-day treatment period, but GTT and ITT results showed that mice receiving H-Exo^{nano} or L-Exo^{nano} PC+ developed glucose intolerance and insulin resistance compared to mice receiving L-Exo^{nano} or H-Exo^{nano} PC- (p<0.05 at 60, 90, and 120 minutes after glucose injection for GTT and at 60 and 90 minutes after glucose injection for ITT). These results suggested that HFD-induced elevations in exosomal PC contributed to insulin resistance and glucose intolerance.

### Example 4

### CD63+A33+ Exosomal PC Modulated the Preferential Targeting of Exosomes to Particular Liver Cell Types

The liver plays a critical role in maintaining glucose homeostasis. Crosstalk between the gut and liver is increasingly recognized due in part to the parallel rise in the incidence of obesity and type II diabetes. However, the role of gut epithelial cell-derived exosomes in the context of liver/gut axis communication has not been investigated.

In order to examine how intestinal epithelial cell-derived exosomes modulate gut-liver communication, a mouse colon epithelial cell line (GFP-MC38) that released GFP- positive exosomes was developed. Exosome size was estimated by nanoparticle tracking analysis and were found to be 110±45 nM. These exosomes were positive for CD63 and A33.

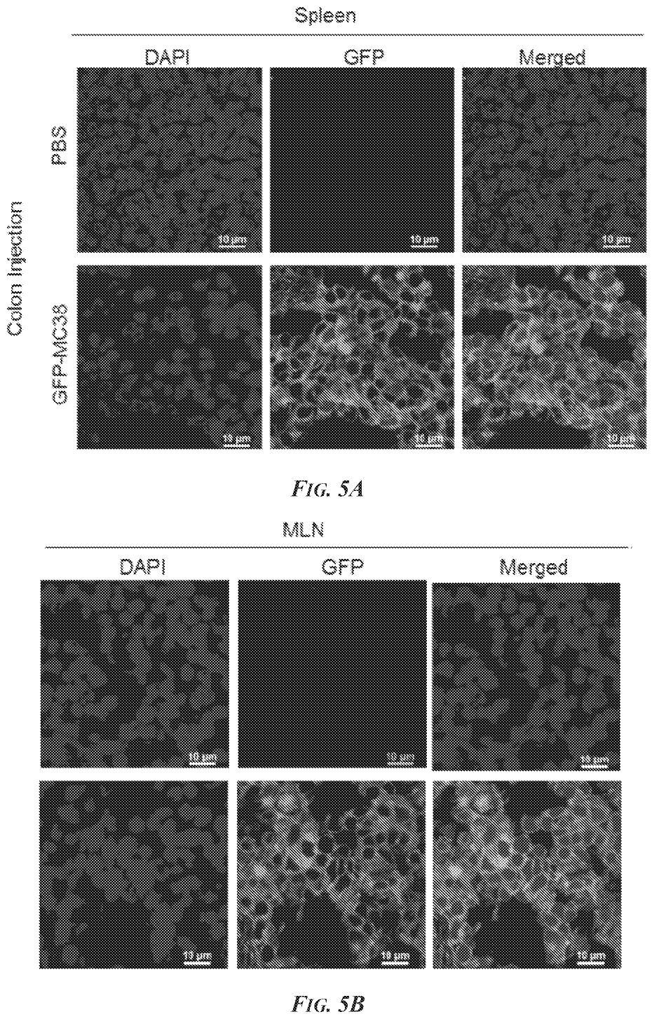

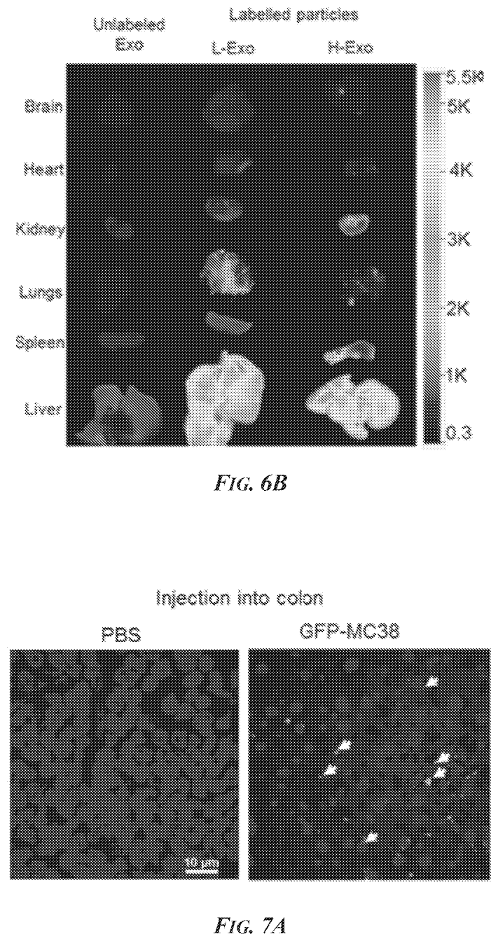

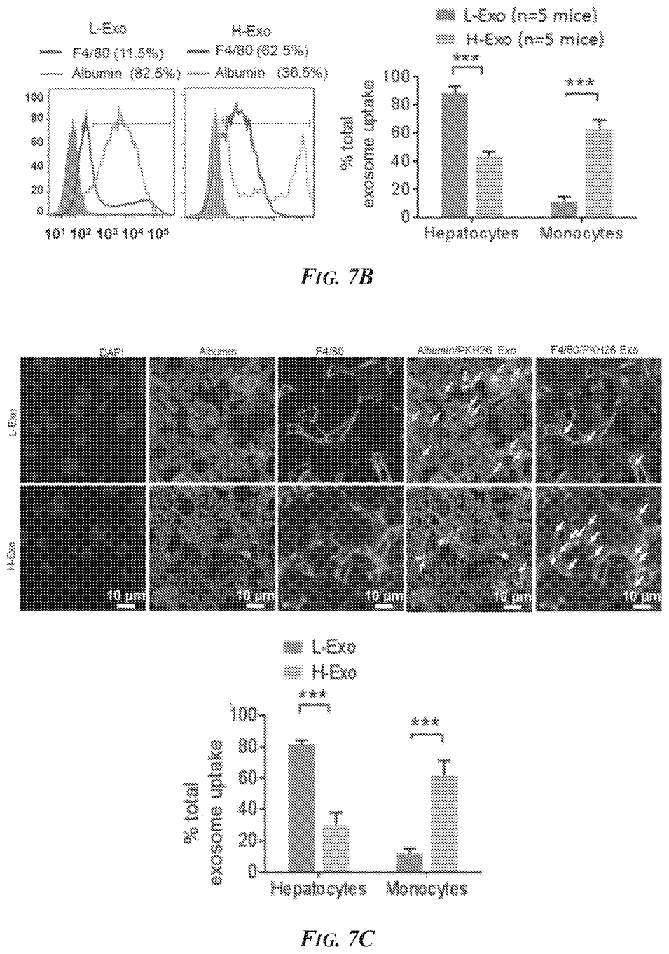

GFP-MC38 cells (5×10⁵) were injected into the colon of C57BL/6 mice. Six weeks post-injection, mice were sacrificed, and organs were harvested. Visualization of liver, spleen, and mesenteric lymph nodes (MLN) with confocal microscopy revealed that the injected GFP-MC38 exosomes indeed reached the liver (FIG. 7A), spleen, and MLN (FIGS. 5A and 5B).

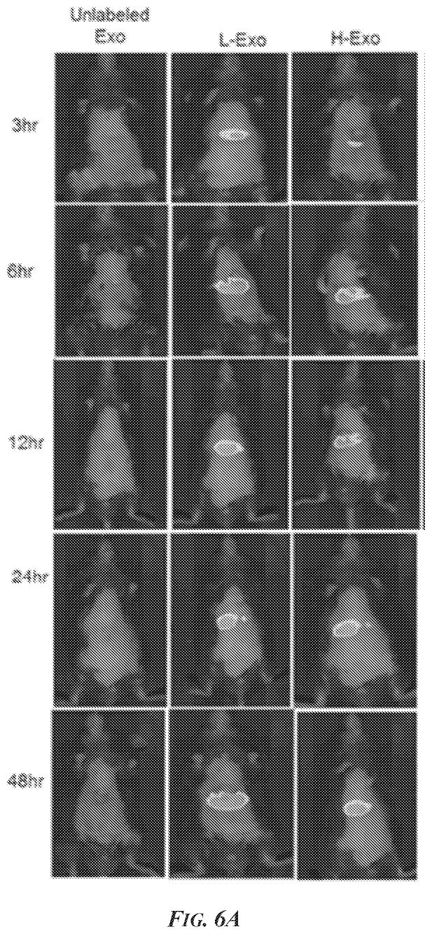

Next, whether endogenous gut epithelial exosomes administered via oral gavage have similar trafficking routes as colon-injected GFP-MC38 exosomes was determined by double labeling H-Exo and L-Exo (2×10⁹ exosomes) with DIR and PKH-26 fluorescent dyes for live mouse imaging at multiple time points (3, 6, 12, 24, and 48 hours; FIG. 6A). Forty-eight hours after oral administration of H-Exo or L-Exo, mice were sacrificed and organs were collected for imaging (FIG. 6B). Scanned organ images suggested that labeled H-Exo or L-Exo trafficked to the liver with the strongest signal compared with the signals detected in other organs.

Exosome recipient cells were identified. Liver cells of the mice imaged as described above were then isolated and stained with anti-albumin (a marker for hepatocytes) and F4/80 (a marker for macrophages or Kupffer cells) and analyzed by flow cytometry. Flow cytometry analysis revealed that the majority of L-Exo (>80%) were taken up by hepatocytes (FIG. 7B, L-Exo panel) and far fewer (˜11%) by F4/80 positive macrophages. By contrast, the majority of H-Exo (>60%) were taken up by F4/80-positive macrophages compared to ˜36.5% uptake by hepatocytes (FIG. 7B, H-Exo panel). These results agreed with those generated with confocal microscopy (FIG. 7C).

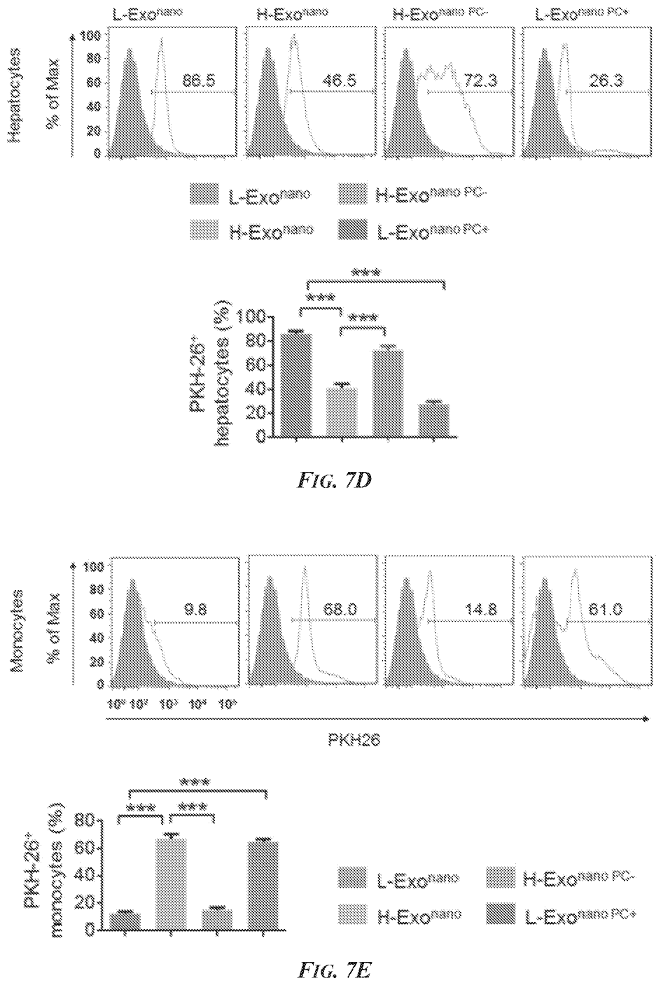

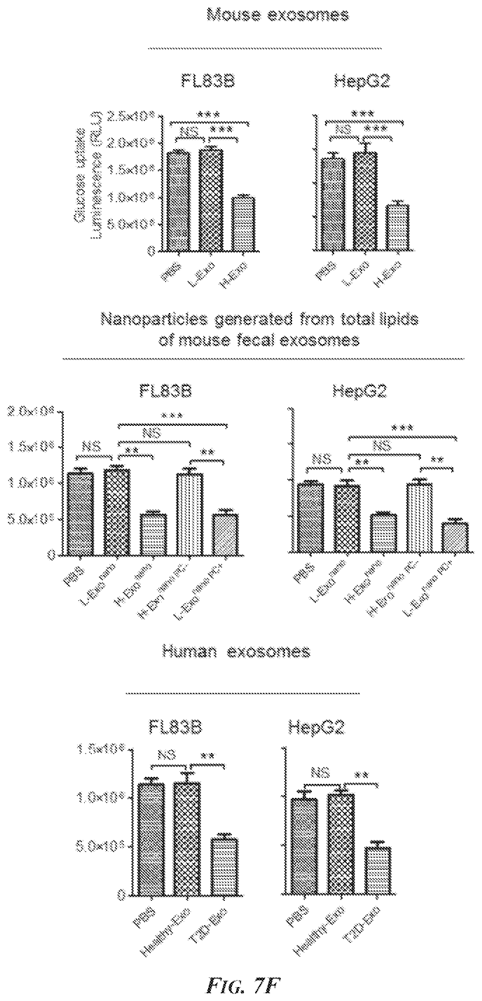

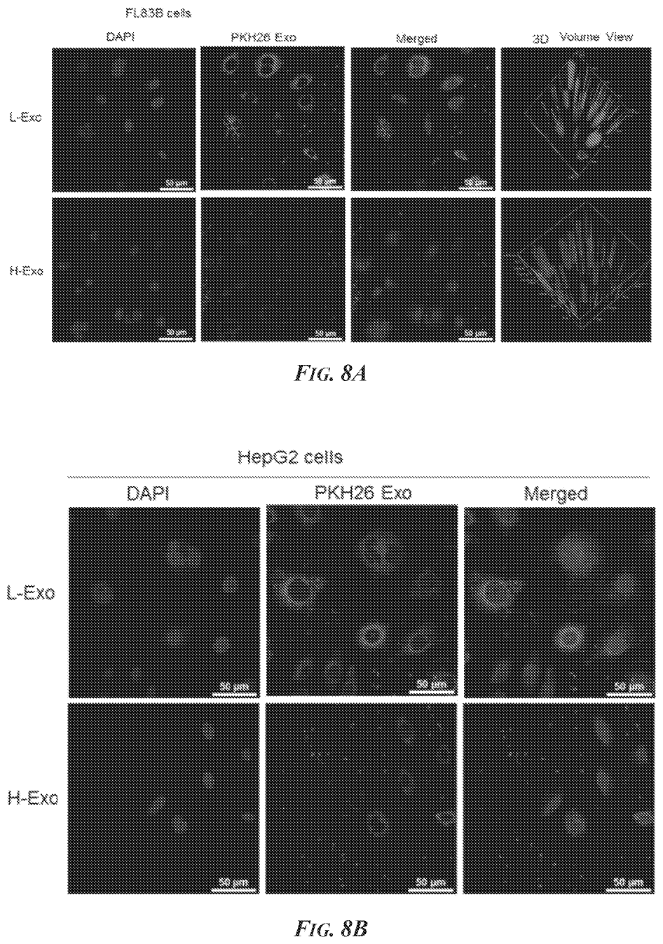

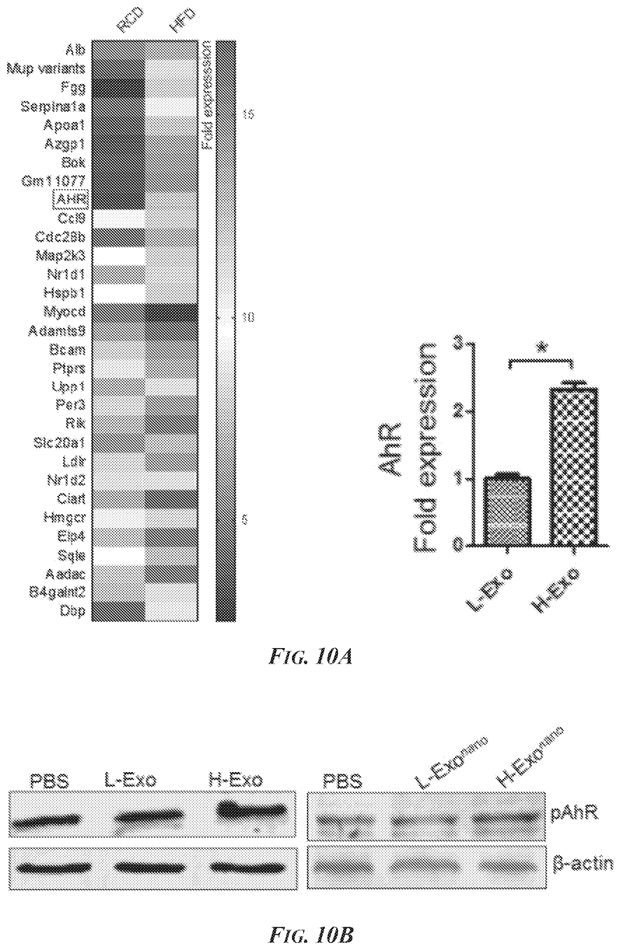

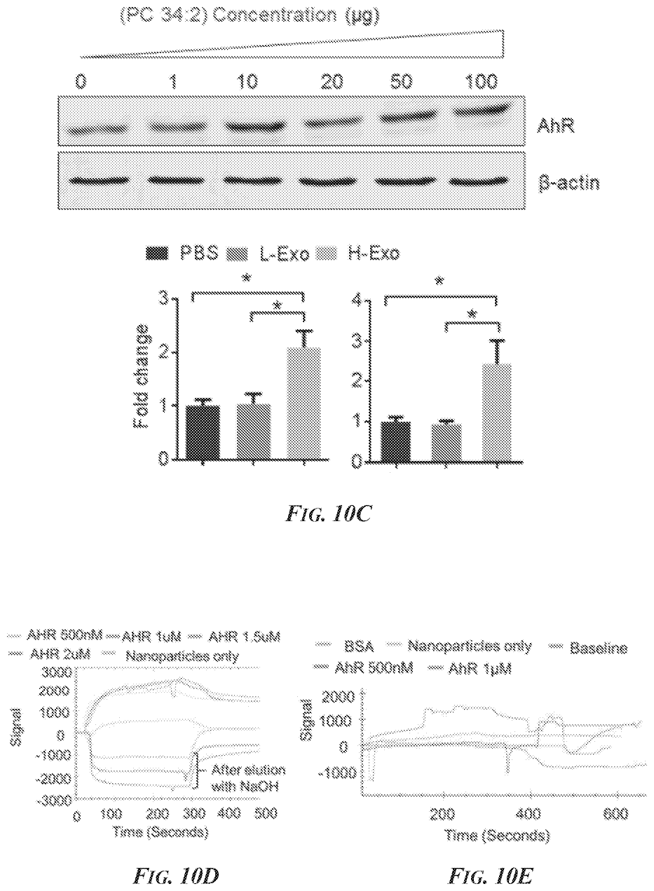

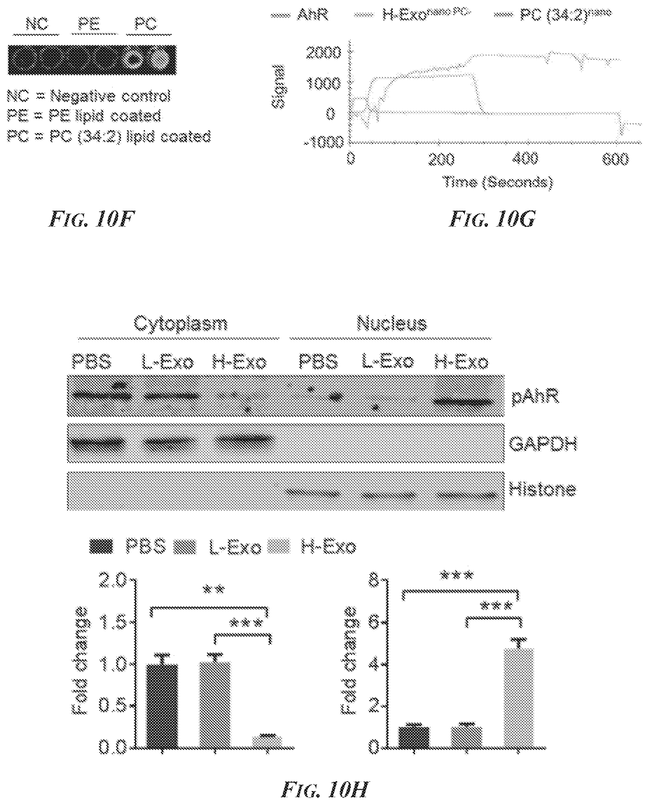

The preferential uptake of L-Exo and H-Exo by different cells was further confirmed using different cell lines in vitro (FIGS. 8A and 8B). Indeed, confocal imaging showed that hepatocytes (mouse FL83B and human HepG2 cells) preferentially took up L-Exo compared to H-Exo, whereas human monocytes (U937 cells) preferentially took up H-Exo compared to L-Exo (FIG. 10B).

Since H-Exo is enriched with PC, whether exosomal PC played a role in mediating the preferential uptake of exosomes by specific cell types was examined by studying the effects of PC depletion from H-Exo. Nanoparticles were prepared with either a depleted band containing PC (PC-) or were supplemented with a known amount of synthesized PC (PC+). Nanoparticles were then labeled with PKH26 dye and co-cultured with hepatocytes (FL83B) and human monocytes (U937) for 16 hours. Flow cytometry results (FIG. 7D) generated following administration of L-Exo^{nano} PC+ indicated that the addition of PC lipids to L-Exo, led to a significant reduction in exosome uptake by hepatocytes (from 86.5% to 26.3%), whereas the removal of PC from H-Exo lipids (H-Exo^{nano} PC-) increased their uptake by hepatocytes (from 46.5% to 72.3%). In monocytes (FIG. 7E), removal of PC from H-Exo led to a reduction in exosome uptake (from 68% to 14.8%), and addition of PC lipid to L-Exo led to an increase in their uptake (from 9.8% to 61%).

These results suggested that the preferential uptake of CD63+A33+ exosomes by particular liver cell types was dependent upon their lipid composition, specifically the percentage of PC lipids they contained.

Once targeting of the liver by gut exosomes was confirmed, whether this had an effect on glucose homeostasis was evaluated. In particular, the effect of exosomes on hepatocyte glucose uptake was evaluated. Inhibition of glucose uptake was observed in vitro in mouse (FL83B cells) and human (HepG2 cells) hepatocytes treated with H-Exo, H-Exo^{nano}, T2D-Exo compared with L-Exo and Healthy-Exo (FIG. 7F). These results suggested that H-Exo, H-Exo^{nano} nanoparticles, and T2D-Exo inhibited glucose uptake by mouse and human hepatocytes.

### Example 5

### H-Exo-Activated Macrophages Induced Release of Inflammatory Cytokines

Pro-inflammatory cytokines can cause insulin resistance in liver by inhibiting insulin signal transduction. Next, whether exosomes had an effect on the cytokine profile was examined. H-Exo vs L-Exo treatment resulted in the induction of numerous inflammatory cytokines detected in the plasma by cytokine array, including TNF-α and IL-6, which are known to contribute to insulin resistance. Increased TNF-α (˜4-fold; p<0.001) and IL-6 (˜3-fold; p<0.01) levels following H-Exo treatment were further confirmed by ELISAs, whereas L-Exo treatment did not result in a statistically significant elevation in either TNF-α or IL-6. Taken together, these findings suggested that the preferential uptake of H-Exo by liver macrophages resulted in macrophage activation and subsequent release of TNF-α and IL-6, thus contributing to the development of insulin resistance.

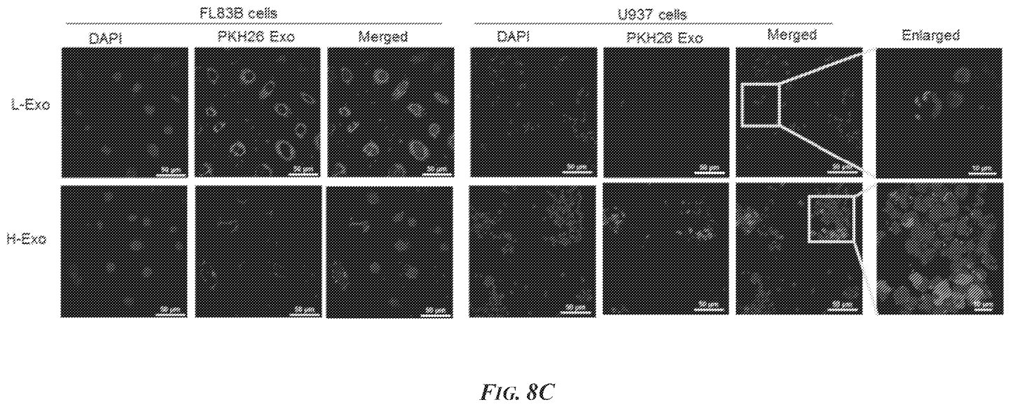

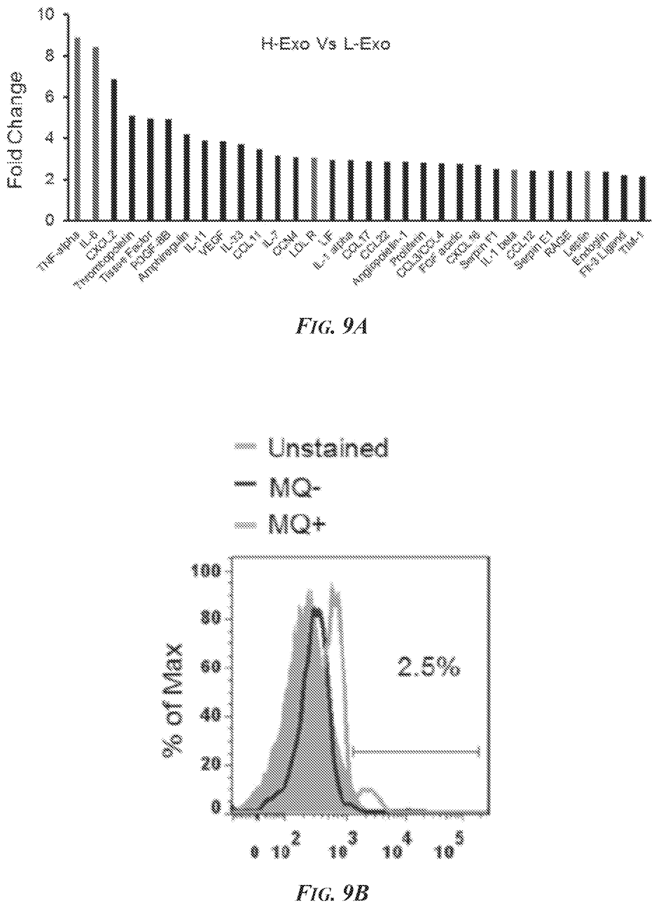

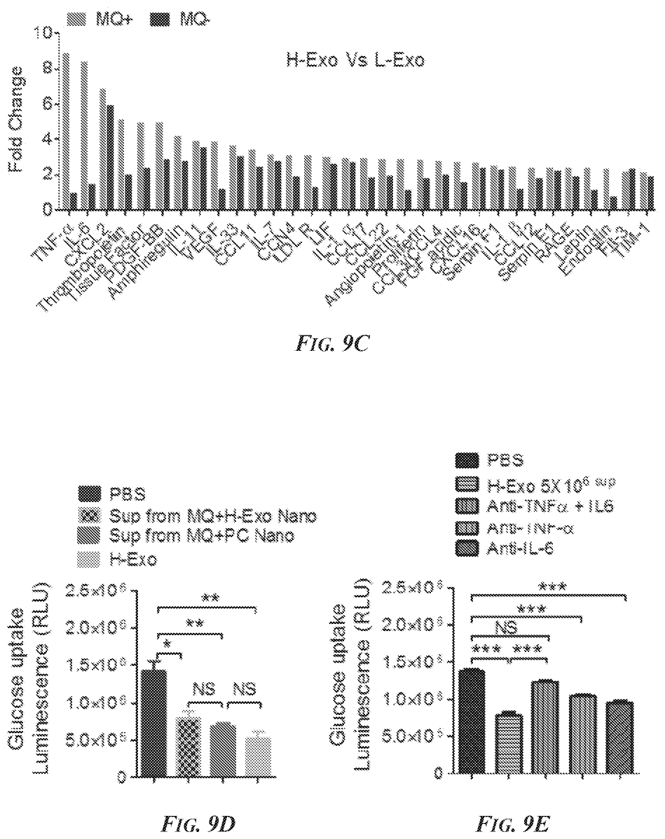

To determine whether macrophages played a role in H-Exo-mediated insulin resistance, macrophages were depleted in mice treated with H-Exo. The effectiveness of the depletion was confirmed by flow cytometry by F4/80 staining in whole blood (FIG. 9B). Depletion of macrophages led to a reduction in TNF-α and IL-6 levels following treatment with H-Exo vs L-Exo (FIG. 9C). Moreover, insulin sensitivity was improved in H-Exo treated mice with depleted macrophages compared with H-Exo treated mice without depletion of macrophages, although the improvement was found to be significantly different at only one time point (p<0.05 at 60 minutes after insulin injection). These results suggested that these macrophage cytokines released in response to H-Exo at least partially contributed to insulin resistance.

Macrophages activation plays a pathogenic role in hepatic insulin resistance. Next, the effects of cytokines from H-Exo- or L-Exo-treated macrophages on hepatocyte glucose uptake was tested. First, the minimum concentration of H-Exo that caused inhibition of hepatocyte glucose uptake was determined. Glucose uptake assay results suggested that a minimum dose of 5×10⁵ H-Exo was required to significantly inhibit glucose uptake (p<0.01 as compared to a PBS-treated negative control at 5×10⁵ H-Exo, and p<0.001 with H-Exo concentrations of 1×10⁶, 2×10⁶, or 5×10⁶). Adding the supernatant from macrophage cultures treated with an elevated dose of H-Exo to hepatocytes further decreased their glucose uptake. However, no reduction in glucose uptake was observed when the supernatant from macrophages treated with 2×10⁶ L-Exo was added to the hepatocytes. Furthermore, nanoparticles generated from H-Exo total lipids (H-Exo^{nano}) and synthesized PC (34:2) had similar impacts on glucose uptake as H-Exo (FIG. 9D). Neutralizing both TNF-α and IL-6 (FIG. 9E) in macrophage supernatants improved glucose uptake, suggesting that macrophage-derived IL-6 and TNF-α played an additive role with H-Exo in inhibiting glucose uptake in hepatocytes.

### Example 6

### AhR Involvement in H-Exo-induced Insulin Resistance

To examine the molecular mechanism underlying H-Exo-mediated insulin resistance and glucose intolerance, AhR-mediated pathways were examined. AhR is a ligand-activated transcription factor that integrates dietary and metabolic cues to control transcriptional programs including insulin-signaling pathway in hepatocytes. Whether H-Exo altered the expression of AhR in mouse livers was thus tested, and indeed the Affymetrix and qPCR results obtained indicated that the gene encoding AhR was upregulated following H-Exo gavage (FIG. 10A). The induction of AhR expression was further confirmed by western blot analysis.

Furthermore, H-Exo and nanoparticles made from H-Exo lipids (H-Exo^{nano}) induced expression of AhR in mouse hepatocytes (FIG. 10B). Induction of AhR in FL83B cells was also confirmed in a PC-dose dependent manner (FIG. 10C). The glucose uptake in mouse hepatocytes treated with PC (34:2), was also inhibited in a PC-dose dependent manner over a two-log difference in PC (34:2) concentration. These results suggested that PC mediated inhibition of hepatic glucose uptake was associated with induction of AhR.

Whether PC bound to AhR was tested. Surface plasmon resonance (SPR) was employed. H-Exo^{nano} (FIG. 10D) and PC (34:2)^{nano} (FIG. 10E) were immobilized on a LIP-1 sensor. Recombinant AhR protein was prepared and run over the immobilized nanoparticles. As shown in FIGS. 10D and 10E, the sensogram of SPR peaks revealed that the AhR protein interacts with H-Exo^{nano} and PC (34:2)^{nano} nanoparticles. Furthermore, PC (34:2) lipid was coated onto ELISA plate and incubated with recombinant AhR protein and subsequently detected by anti-AhR antibody (FIG. 10F). Then, PC binding to AhR was demonstrated by immobilizing recombinant AhR on the NTA chip (protein sensor). H-Exo^{nano} PC- and PC (34:2)^{nano} were run over the immobilized AhR (FIG. 10G).

Whether H-Exo treatment resulted in increased AhR activation (phosphorylation) was also tested. Phosphorylated AhR (pAhR) was increased in the nucleus of H-Exo treated hepatocytes (FIG. 10H, while no induction of pAhR was observed when the hepatocytes treated were with L-Exo and PBS.

Using AhR-deficient (AhR^{-/-}) mice, the roles of AhR in H-Exo-mediated insulin resistance were tested. In AhR^{-/-} mice, H-Exo did not impair glucose tolerance or insulin responsiveness, unlike its effects in wild-type C57BL/6 mice. Consistent with these results, there was no difference in glucose uptake in AhR^{-/-} mouse hepatocytes treated with H-Exo vs L-Exo (FIGS. 10I and 10J). These results suggested that H-Exo PC contributed to insulin resistance via overexpression and subsequent activation of AhR.

### Example 7

Overactivation of AhR Led to Downregulation of IRS-2 Expression and Contributed to Insulin Resistance

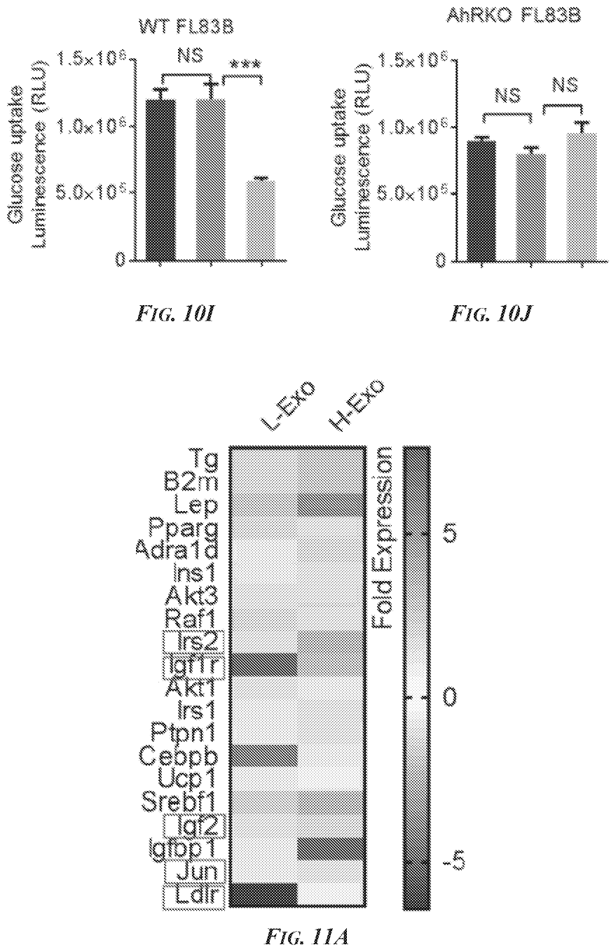

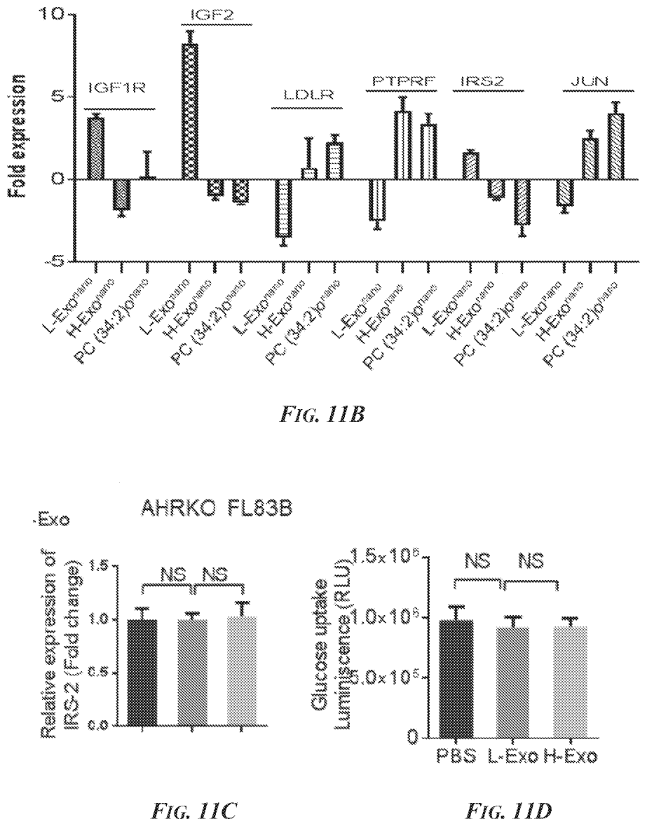

To understand the mechanism underlying H-Exo-induced and AhR-mediated insulin resistance, an insulin signaling array (Qiagen) was employed to quantitatively analyze the expression of genes associated with the insulin pathway in mouse hepatocytes. Array data revealed that the expression of several genes was altered in hepatocytes treated with H-Exo vs L-Exo (FIG. 11A, highlighted by boxes). Among those genes, IRS2 expression was >4-fold lower in H-Exo-treated hepatocytes, and this result was confirmed by qPCR in mouse hepatocytes treated with nanoparticles from total lipids of H-Exo and L-Exo and nanoparticles from PC (34:2) only (FIG. 11B).

IRS-2 is especially important in hepatic nutrient homeostasis. Mice lacking the IRS2 gene develop diabetes due to peripheral insulin resistance and display many of the hallmarks of type 2 diabetes in human subjects. IRS-2 is directly phosphorylated by the insulin receptor, which leads to the recruitment and activation of downstream signaling proteins. Activation of IRS-2 was tested by measuring pIRS-2 levels by western blot analysis, the results of which suggested that the PC in H-Exo played a role in inhibiting the expression as well as activation of IRS-2.

Treatment of AhR^{-/-} mouse hepatocytes with H-Exo or L-Exo did not inhibit IRS2 expression (FIG. 11C), indicating that IRS2 inhibition occurred via the AhR receptor-mediated pathway.

Finally, to determine if IRS-2 was involved in H-Exo-mediated inhibition of glucose uptake, IRS-2 was overexpressed in hepatocytes. No inhibition of glucose uptake was observed in H-Exo treated hepatocytes (FIG. 11D), suggesting that IRS2 is an essential gene for H-Exo-mediated inhibition of glucose uptake.

### Example 8

H-Exo-Mediated Activation of AhR Led to Mouse Dyslipidemia

AhR is known to be involved in cholesterol synthesis, and high-fat intake induces insulin resistance and dyslipidemia including high cholesterol and triglyceride levels. Plasma cholesterol levels in HFD-fed mice treated with mouse fecal exosomes (L-Exo and H-Exo) for 14 days were determined. H-Exo-treated C57BL/6 and C57BL/6 germ-free mice showed significantly elevated levels of plasma cholesterol and triglycerides, whereas AhR^{-/-} mice showed no significant change in plasma cholesterol and triglycerides (FIG. 12). Furthermore, plasma ALT and AST levels were significantly elevated in H-Exo treated mice vs PBS and L-Exo treated mice. Collectively, these results suggest H-Exo causes dyslipidemia via activation of AhR-mediated signaling.

### Materials and Methods for Examples 9-18

Mice. 6- to 8- week-old male C57BL/6 mice were purchased from the Jackson Laboratory (Bar Harbor, Me.) and maintained on a 12-h/12-h light/dark cycle in a pathogen-free animal facility at the University of Louisville. AhR knockout mice were purchased from Taconic Biosciences (Rensselaer, N.Y.). Germ-free mice were purchased from the National Gnotobiotic Rodent Resource Center (University of North Carolina, Chapel Hill, N.C.) and maintained in flexible film isolators (Taconic Farm) at the clean mouse facility of the University of Louisville. Animal care was performed following the Institute for Laboratory Animal Research (ILAR) guidelines, and all animal experiments were conducted in accordance with protocols approved by the University of Louisville Institutional Animal Care and Use Committee (Louisville, Ky.).

Human subjects. The study involved fourteen healthy volunteers between the ages of 25 to 45 years (all males), seven obese (age matched) and fourteen Type 2 diabetes (T2D) patients. No healthy volunteers had a history of chronic gastrointestinal disease. Seven healthy volunteers and seven T2D patients were recruited from patients in the outpatient endocrinology clinic at University of Louisville Hospital, Louisville, Ky., USA. Seven obese and seven T2D patients clinical fecal samples were collected from patients in the Department of Surgery, Huai'an First People's Hospital, Huai'an, Jiangsu, China. Type 2 diabetes was diagnosed according to the American Diabetes Association diagnostic criteria (American Diabetes Association 2012). All participants were educated regarding their participation and signed a written consent form. Approval for the study was granted by the University of Louisville Research Ethics committee.

Cells. Murine colon (MC-38), human colon (Caco-2) and human embryonic kidney 293T (American Type Culture Collection, ATCC) cells were grown in tissue culture plate/dishes with Dulbecco Modified Eagle Medium (DMEM, Thermo Fisher Sci.) supplemented with 10% heat-inactivated fetal bovine serum (FBS), 100 U ml⁻¹ penicillin, and 100 mg ml⁻¹ streptomycin at 37° C. in a 5% CO² atmosphere. For murine hepatocytes (FL83B) (American Type Culture Collection, ATCC) were grown in tissue culture plates with F 12K medium (Thermo Fisher Sci) supplemented with 10% heat-inactivated fetal bovine serum (FBS), 100 U ml⁻¹ penicillin, and 100 mg ml⁻¹ streptomycin at 37° C. in a 5% CO₂ atmosphere.

Generation of knock out cells. Lentivirus were prepared to generate knockout for Foxa2, VAMP7, AhR and Akt-1. In brief, HEK293T cells were transfected with three plasmids, pCMVdelta8.2 (5 μg) and VSV-G (1 μg) along with 6 μg of specific gene knock out plasmid (CRISPR/CAS9) using the 30 μl of P3000 in 500 μl of Opti-MEMO® in tube 1. Tube 2 contained 500 μl of Opti-MEMO® plus 22 μl L3000 from transfecting kit (cat. no. L3000-015, Invitrogen, USA). The contents of the tubes were mixed and incubated at RT for 15 minutes. The mixture was added to 100 mm dish of HEK293T (50-60% confluent). Pseudovirus-containing culture medium was collected after 72 h of transfection, and the viral titer was estimated. MC-38 cells (2×10⁵) in a six-well plate received 10 μg ml⁻¹ of puromycin as well as an appropriate amount of viral stocks in the medium. After selection by puromycin, results were confirmed by qPCR and western blot. All plasmids were purchased from Santa Cruz Biotechnology (see Table 5).

TABLE 5
List of CRISPR/Cas9 Plasmids Employed

| Target | Santa Cruz Biotechnology Catalog No. |
|---|---|
| Foxa2 | sc-420890 |
| AHR | sc-419054 |
| VAMP7 | sc-423230 |
| Akt-1 | sc-419071 |

Isolation and purification of Ginger derived nano-particles (GDNP). Hawaiian ginger roots were purchased from local market and skin was peeled out manually. After that, ginger was chopped into small pieces and blended in the blender and collected juice was diluted in PBS, differentially centrifuged (500 g for 10 min, 2,000 g for 20 min, 5,000 g for 30 min, 10,000 g for 1 h) and the nano-particles then purified on a sucrose gradient (8, 30, 45 and 60% sucrose in 20 mM Tris—Cl, pH 7.2). The band settled at 30% sucrose was re-purified by washing with PBS. The purified GDNPs were prepared for transmission electron microscope (TEM) using a conventional procedure and observed using an FEI Tecnai F20 sent to electron microscope facility equipped at UAB (University of Alabama, Alabama, USA). The electron micrographs were taken at the following settings, 80 kV at a magnification of 15,000 and defocus of 100 and 500 nm. The size and concentration of GDNP was estimated by NS300 (Malvern Panalytical, UK).

Endotoxin detection in fecal exosomes. Endotoxin in fecal exosomes was detected using a PIERCE™ brand Chromogenic Endotoxin Quant kit (Cat. No. A39552S). In brief, all reagents were prepared according to the manufacturer's instructions. 50 μl of endotoxin standards or test samples were added per well (triplicates) of the plate. 50 μl of amebocyte lysate reagent was added into each well, mixed and the plate incubated for 30 min at 37° C. 100 μl of pre-warmed chromogenic substrate was added into each well and vigorously mixed and the plate incubated for 6 min at 37° C. 50 μl of stop solution (25% acetic acid) was added and mixed. The plate was read for optical density at 405 nm. A standard graph was plotted and calculations for endotoxin in test samples was done accordingly.

Bio distribution targeting of orally administrated GDNP by live imaging and confocal microscopy. After 6 h of orally administering 50 mg of either DiR or PKH26 fluorescent dye (Sigma) labelled GDNP, mice were sacrificed and small intestine, colon, MLN, spleen and liver tissues were used for imaging. DiR fluorescent signal was detected and measured using the Imaging Station Pearl Impulse (Li-COR Biosciences). The labeled GDNP in the gut of mice were visualized using an Odyssey CLx Imaging System (Li-COR Biosciences). PKH26 signal in frozen tissue sections was observed using the confocal laser scanning microscopy system (Nikon, Melville, N.Y.).

Nanoparticle tracking analysis. Sucrose purified nanoparticles samples were analyzed for particle concentration and size distribution using the nanoparticle tracking analysis method provided by the Malvern NanoSight NS300 (Malvern Instruments Ltd, Malvern, United Kingdom). The assays were performed in accordance with the manufacturer's instructions. Briefly, for the NanoSight, three independent replicates of diluted particles preparations in PBS were injected at a constant rate into the tracking chamber using the provided syringe pump. The samples were tracked at room temperature for 60 seconds. Shutter and gain were manually adjusted for optimal detection and were kept at optimized settings for all samples. The data were captured and analyzed with NTA Build 127 software (version 2.2, Malvern Instruments Ltd, Malvern, UK).

High fat diet mouse model. 6 to 8-week-old C57BL/6 male mice (n=10 per group) were fed either regular chow diet (RCD; 10% Fat) or high fat diet (HFD; 60% Fat). One HFD fed group was treated along with PBS and another HFD group along with GDNP (6×10⁸ mL^{-l}) by adding into drinking water for at least 12 months or entire lifespan. The glucose and insulin tolerance test (GTT & ITT) were performed at 3, 6, 9 & 12 months after treatment. A more detailed description of high fat diet employed is presented in Table 6.

TABLE 6
Composition of the High Fact Diet

| Class description | Ingredient | Grams |
|---|---|---|
| Protein | Casein, Lactic, 30Mesh | 200 |
| Protein | Cystine, L | 3 |
| Carbohydrate | Lodex 10, | 125 |
| Carbohydrate | Fine granulated Sucrose | 72.8 |
| Fiber | Solka Floc, FCC200 | 50 |
| Fat | Lard | 245 |
| Fat | Soybean oil, USP | 25 |
| Mineral | S10026B | 50 |
| Vitamin | Choline bitartrate | 2 |
| Vitamin | V10001C | 1 |
| Dye | Dye Blue FD&C #1, Alum. Lake 35-42% | 0.05 |

Lipid extraction from GDNP. Total lipids were extracted from sucrose purified/washed band of processed ginger derived nano-particles. Briefly, 1.9 ml 2:1 (v/v) methanol:chloroform was added to 0.5 ml of GDNPs in PBS and 0.625 ml of chloroform and water were added sequentially and vortexed thoroughly. The aqueous and organic phase were separated by centrifugation at 2,000 r.p.m. for 10 min at 22° C. in glass tubes. Organic phase was collected by a glass pipette and dispensed into fresh glass tubes. The organic phase was dried under nitrogen (2 psi). Total lipids were determined using the phosphate assay.

Lipidomic analysis with mass spectrometry (MS). Extracted total lipids from GDNP or lipid band 1 (LB1) were submitted to the Lipidomics Research Center, Kansas State University (Manhattan, Kans.) for analysis using MS. In brief, the lipid composition was determined using triple quadrupole MS (Applied Biosystems Q-TRAP, Applied Biosystems, Foster City, Calif.). The data are reported as the concentration (nmol) and percentage of each lipid within the total signal for the molecular species determined after normalization of the signals to internal standards of the same lipid class.

Thin-layer chromatography (TLC) analysis. Total lipids from GDNP were quantitatively analyzed using a method previously described and used for TLC analysis. Briefly, HPTLC-plates (silica gel 60 with a concentrating zone, 20 cm×10 cm; Merck) were used for the separation. After extracting samples of concentrated lipid from GDNP, the lipids (PA and PC from Avanti Polar Lipids, Inc. were used as standards) were separated on a plate that had been developed with chloroform/methanol/acetic acid (190:9:1, by vol). After drying in air, the plates were stained either by iodine powder fumes or sprayed with a 10% copper sulfate and 8% phosphoric acid solution and then charred by heating at 120° C. for 12 min or until bands were developed. The lipid bands on the plate were imaged using an Odyssey Scanner (Licor Bioscience, Lincoln Nebr.).

Nanoparticles preparation from lipid extracted from GDNP. To prepare GDNP nanoparticles, GDNP derived lipids were extracted with chloroform and dried under vacuum. 300 nmol of lipid was suspended in 600 μl of 155 mM NaCl with or without microRNA (miR-375) or scramble RNA (20 nm each). 4 μl of PEI was added. Ultra-sonicate the mixture/solution for 20 minutes in bath sonicator (FS60 bath sonicator, Fisher Scientific, Pittsburg, Pa.) at 4° C. After sonication, nanoparticles were pellet down by ultracentrifuge for 1 hour at 100,000g. Before being used in experiments, the nanoparticles were homogenized by passing the samples through a high-pressure homogenizer (Avestin Inc., Ottawa, Canada) using a protocol provided in the homogenizer instruction manual.

Affymetrix mRNA microarray. Total RNA was extracted from tissues using Qiagen RNeasy mini kit (Cat. no. 74104). 100 ng of RNA for each sample submitted to Invitrogen/ThermoFisher Scientific Affymetrix facility, Santa Carla, Calif., USA. Transcriptome Analysis Console (TAC) 4.0 from ThermoFisher Scientific was used to analyze the data.

Surface plasmon resonance (SPR). SPR experiments were conducted on an OPENSPR™ (Nicoya, Lifesciences, ON, CA). Experiments were performed on a LIP-1 sensor (Nicoya, Lifesciences). Tests were run at a flow rate of 20 μl/min using BBS running buffer (20 mM HEPES, 150 mM NaCl, pH 7.4). First, the LIP-1 sensor chip was cleaned with octyl -D-glucopyranoside (40 mM) and CHAPS (20 mM). Nanoparticles (1 mg/ml) were injected on the sensor chip for 10 min until stable resonance was obtained. After immobilization of nanoparticles, the surface was blocked with BSA (3%) in running buffer. After a stable signal was obtained, recombinant human Foxa2 protein (Cat. no. ab95848; Abcam, USA) or synthesized peptides (GenScript Biotech, USA) was run over the immobilized liposomes. A negative control was also performed by injecting protein onto a blank sensor chip to check for non-specific binding. After 10 min, the nanoparticles binding protein were eluted using NaOH (200 μM). The sensograms were analyzed using TraceDrawer kinetic analysis software.

Cytoplasmic and nuclear protein extraction. To prepare nuclear protein extracts, MC-38 cells were washed with a cold PBS. After washing with cold PBS for 4 min, the cell pellets were re-suspended in cold cytoplasmic extract buffer (10 mM HEPES, 60 mM KCl, 1 mM EDTA, 1 mM DTT and 1 mM PMSF, pH 7.6) containing 0.075% (v/v) NP40. After incubating on ice for 3 min, the cell suspension was centrifuged at 400 g for 4 min, the supernatant (cytoplasmic protein) was collected and the pellet was washed with cytoplasmic extract buffer without NP40 one more time. Nuclear protein was extracted from the pellet with nuclear extract buffer (20 mM Tris Cl, 420 mM NaCl, 1.5 mM MgCl₂, 0.2 mM EDTA, 1 mM PMSF and 25% (v/v) glycerol, pH 8.0). The proteins were quantified.

Quantitative reverse transcription polymerase chain reaction (qPCR) analysis mRNA expression. Total RNA was isolated from tissue and cells using RNeasy mini kit (Qiagen). For analysis of Foxa2, VAMP7, AhR, and IRS-1 & 2 mRNA expression. 1 μg of total RNA was reverse transcribed using SuperScript III reverse transcriptase (Invitrogen) and quantitation was performed using primers (Eurofins) with QuantiTect SYBR Green PCR (Qiagen). GAPDH was used for normalization. The primer sequences are listed in Table 7. qPCR was run using the BioRad CFX96 qPCR System with each reaction run in triplicate.

TABLE 7
Primer Sequences Employed

| Target* | Sequence (5′-3′) |
|---|---|
| Foxa2F | CCCTACGCCAACATGAACTCG; SEQ ID NO: 15 |
| Foxa2R | GTTCTGCCGGTAGAAAGGGA; SEQ ID NO: 16 |
| AhR F | GCAATAGCTACTCCACTTCAG; SEQ ID NO: 1 |
| AhR R | GGTGTGAAGTCTAGCTTGTG; SEQ ID NO: 2 |
| VAMP7 F | TCAAGAGCACAGACAGCACTTCC; SEQ ID NO: 17 |
| VAMP7 R | GCCATGTAAATCCACCACAGAGAG; SEQ ID NO: 18 |
| Bmal1 F | CCAAGAAAGTATGGACACAGACAAA; SEQ ID NO: 19 |
| Bmal1 R | GCATTCTTGATCCTTCCTTGGT; SEQ ID NO: 20 |
| Pri-miR375F | GCTCCGCCTCCATGAGTCAATA; SEQ ID NO: 21 |
| Pri-miR375R | CACGCGAGCCGAACGAACAA; SEQ ID NO: 22 |
| pGL3Mlu1 F | GTACGCGTCCCACATGTGTTCACCAGCA; SEQ ID NO: 23 |
| pGL3Nco1 R | GAGGTACCCCGGAGCGGAAGACCC; SEQ ID NO: 24 |
| Mut 375PromF | GTGTGCTCCGCCTCCACAAGCCACGATTTGCCCCGAGCAAA; |
| | SEQ ID NO: 25 |
| Mut 375PromR | TTTGCTCGGGGCAAATCGTGGCTTGTGGAGGCGGAGCACAC; |
| | SEQ ID NO: 26 |
| E. coli tnaA F | TGCAACCATCACCAGTAAC; SEQ ID NO: 27 |
| E. coli tnaA R | GTCCATTACCACCGGAATATC; SEQ ID NO: 28 |
| CyP7a1 F | AGCAACTAAACAACCTGCCAGTACTA; SEQ ID NO: 29 |
| CyP7a1 R | GTCCGGATATTCAAGGATGCA; SEQ ID NO: 30 |
| miR-375 F | TTTGTTCGTTCGGCTCGCGTGA;; SEQ ID NO: 31 |

*F: forward primer; R: reverse primer.

miRNA PCR microarray. Total RNA contained small RNA was isolated from tissue and cells using a miRNeasy mini kit (Qiagen; cat. no. 217004). miRNA expression profiling for exosomes was performed using the Qiagen miScript miRNA PCR Array Mouse miRBase Profiler (Cat. No. 331223) using an Applied Biosystems ViiA 7 Real-Time PCR System. Normalization to endogenous control genes included SNORD61, SNORD68, SNORD72, SNORD95, and RNU6 to correct for potential RNA input or RT efficiency biases. miRNA data generated from exosomes were comparatively analyzed by the online free data analysis software at https://dataanalysis.qiagen.com. Quantile normalization and subsequent data processing were performed and scatter plots representing differentially regulated miRNAs were generated.

BLASTN analysis. Basic Local Alignment Search Tool was used for sequence match as online available via the website for the National Center for Biotechnology Information of the National Institutes of Health.

Chromatin immunoprecipitation (ChIP) Assay. Nuclear extracts from MC-38 cells (5×10⁶), prepared, pull down assay performed with either Foxa2 antibody or IgG control antibody by using the R&D Systems ChIP protocol (Minneapolis, Minn., United States of America). In brief, the cells were incubated in 1% formaldehyde on a rocker shaker for 15 minutes at RT. 125 mM of glycine was added to quench the formaldehyde. Cells were pelleted down and remove the media. Cells were re-suspended in 500 μL of Lysis Buffer per 5×10⁶ cells containing protease inhibitors (10 μg/mL Leupeptin, 10 μg/mL Aprotinin, and 1 mM PMSF) followed by 10 minutes incubation on ice. Samples were sonicated to shear the chromatin and transfer 500 μL of each sample to a 1.5 mL micro-centrifuge tube and centrifuge the lysates for 10 minutes using ultracentrifuge at 12,000 g at 4° C. Supernatant was collected in a clean tube. Supernatant was diluted into 1 mL of dilution buffer and 5 μg of the antibody or normal IgG were added to the samples followed by 15 minutes incubation at RT or overnight at 4° C. Secondary antibody (biotinylated) was added and incubate at RT for 15 minutes. 50-60 μL of Streptavidin beads (Dyna beads) were added to the samples and rotate for 30 minutes at 2-8° C. on a rotator. Beads were pelleted by centrifugation at 12000 g for 1 minute. Beads were washed for four times with pre-chilled wash buffer. 100 μL of chelating resin solution were added to the beads and mixed well. Samples were boiled for 10 minutes using heat block. Centrifuge the suspension at 12000 g for 1 minute and transfer the supernatant to a clean tube. DNA was cleaned up by DNA purification kit (K182104A, Thermofisher, scientific) and eluted into 50 μL of deionized or distilled water. Eluted DNA was used as template for miR-375 or Foxa2 promoter region amplification by PCR by using designed primers (Table 7).

Transfections of constructs and luciferase assay. MC-38 cells were transfected with 500 ng of pGL3, pGL3miR375 or mutated constructs using kit from Invitrogen (Cat. No. L3000-015) in accordance with manufacturer's instructions. Details were described in generation of knock out cells section. pGL3-miR375 or pGL3 or Mut- pGL3-miR375 plasmids transfected wild type (WT) or Foxa2KO MC-38 cells were treated with PBS or GDNP for 16 hours at 37° C. in a CO₂ incubator. Luciferase activity was measured using dual luciferase system (Cat. No. E1910) from Promega Corp., Madison, Wis., USA as per manufacturer's instructions. In case of miRNA transfection, MC-38 cells were transfected with 20 nm miRNA (miR-375 and biotinylated miR-375) by using RNAiMAX (Invitrogen). Cells were incubated for 72 h at 37° C. in a CO₂ incubator. For pull-down of biotinylated miR-375 was performed with streptavidin antibody. Pulled products were used for western blot.

Glucose and Insulin tolerance tests (GTT & ITT). For glucose tolerance tests, after an overnight fast, baseline glucose levels were determined using the glucometer (Priology, USA). Then, mice were given an intraperitoneal injection of glucose (dextrose) at a dose of 2 mg g⁻¹ of body weight. The blood glucose levels were measured at 30, 60, 90, and 120 minutes after glucose injection. For insulin tolerance tests, mice were fasted for 6 h and basal blood glucose levels were determined. Then mice were given an intraperitoneal injection of insulin (1.2 units g⁻¹ of body weight). The blood glucose levels were measured at 30, 60, and 90 minutes (otherwise indicated in the Figures) after insulin injection.

Labelling of nanoparticles. Nanoparticle were labeled with DIR or PKH26 Fluorescent Cell Linker Kits (Sigma) using the manufacturer's instructions. Nanoparticle were suspended in 250 μl of diluent C with 4 μl of DIR or PKH26 dye and subsequently incubated for 30 min at room temperature. After washing with PBS and centrifugation at 10,000g for 1 h at 4° C., the pellet was washed twice to remove unbound dye and finally re-suspended in PBS and used in experiments.

Bacteria GDNP nanoparticles uptake assay. Briefly, 1×10⁷ E. coli cells were incubated for 60 min at room temperature with 1 mg of PKH26-labeled nanoparticles or 500 ng of miR-375/scrambled RNA encapsulated in nanoparticles. After two washes with PBS, E. coli uptake of nanoparticles was visualized using a confocal microscope. To exclude the possibility of detecting nanoparticles remaining (adhering) on the outside of bacteria, the bacteria were washed three times with medium and treated with 100 μl of 0.5% Triton X-100 for eight minutes, followed by the immediate addition of bacteria broth to wash bacteria 2× before the bacteria were imaged using confocal microscopy. (Note: 0.5% Triton X-100 did not affect bacterial viability for at least 30 minutes after addition).

Western blot analysis. The tissues were washed with ice cold PBS and homogenized. The cells were lysed in radio-immunoprecipitation assay (RIPA) lysis buffer with addition of protease inhibitor for 1 h at 4° C. The crude lysates were centrifuged at 14,000 g for 15 min. Protein concentrations were determined using the BioRad Protein Assay Reagent. Samples were diluted in 1× SDS sample buffer. Proteins were separated by 10-12% or gradient SDS-PAGE and transferred to nitrocellulose membranes (Bio-Rad). Individual protein was detected with specific antibodies and visualized by infrared fluorescent secondary antibodies (Table 8). The protein bands were visualized and analyzed on an Odyssey CLx Imager (LiCor Inc, Lincoln, Nebr.).

TABLE 8
List of Antibodies Employed

| S. | | | | |
|---|---|---|---|---|
| No. | Target | Application | Source | Cat. No. |
| 1 | Foxa2 | ChIP | Thermo Scientific | 701698 |
| 2 | Foxa2 | IF/Western | R&D Systems | MAB2400 |
| 3 | pFoxa2 | IF/WB | Thermo Scientific | 710680 |
| 4 | pAhR | IF/WB | Thermo Scientific | PA5-38404 |
| 5 | AHR | WB | Santa Cruz | Sc133088 |
| 6 | VAMP7 | IF/WB | Cell Signaling | 13876S |
| 7 | CD63 | IF | Novus Biologicals | NBP2-32830 |
| 8 | A33 | IF/Flow | Biorybt | Orb15687 |
| 9 | pAkt-1 | IF/WB | Cell Signaling | 9018S |
| 10 | pAkt-2 | IF/WB | Cell Signaling | 8599S |
| 11 | B-Actin | WB | Santa Cruz | Sc47778 |
| 12 | IgG | Pull down | Santa Cruz | Sc65662 |
| 13 | GAPDH | WB | Santa Cruz | Sc47724 |
| 14 | F4/80 | Flow cytometry | eBioscience | 11-4801-82 |
| 15 | F4/80 | IF | eBioscience | 14-4801-82 |

Flow cytometry. The liver of mice was perfused with perfusion buffer ((Ca²⁺-Mg²⁺ free HBSS containing 0.5 mM EGTA, 10 mM HEPES and 4.2 mM NaHCO₃ supplemented with Type I collagenase (0.05%) and trypsin inhibitor (50 μg/ml; pH 7.2)) and then harvested into complete medium. Cells isolated from liver tissue were fixed with 2% paraformaldehyde (PFA) and stained with albumin and F4/80 primary antibodies for 40 min at 4° C. After three washes with PBS, cells were stained with Alexa488 or PE conjugated secondary antibodies for 1 h at RT. Stained liver cells (monocytes and hepatocytes) treated with PKH26⁺ nanoparticle or fecal exosomes were acquired using a BD FACSCanto flow cytometer (BD Biosciences, San Jose, Calif.) and analyzed using FlowJo software (Tree Star Inc., Ashland, Oreg.). For sorting of bacteria, feces samples form mice gavaged with labeled exosomes (after 6 h) were re-suspended with PBS and centrifuged at 2000 g for 10 minutes to remove big pellets. Suspension from this step was used for sorting. PKH26 positive bacteria were sorted by BD FACSARIA™ III instrument equipped with 488 and 633nm laser.

Confocal microscopy. For frozen sections, periodate-lysine-paraformaldehyde (PLP) fixed tissues were dehydrated with 30% sucrose in PBS overnight at 4° C. and embedded into optimal cutting temperature (OCT) compound. Tissue was subsequently cut into ultrathin slices (5 μm) using a microtome. The tissue sections were blocked with 5% bovine serum albumin (BSA) in PBS. Primary antibodies (1:800) were added and incubated at 4° C. overnight. Sections were washed three times followed by secondary antibodies conjugated to a fluorescent dye (at 1:2000 dilution). Nuclei were stained with 4′, 6-diamidino-2-phenylindole dihydrochloride (DAPI). For in-vitro cultured cells, 2×10⁵ cells were grown on coverslips in six well plates and co-cultured with PKH26 labeled feces exosomes for 16 h at 37° C. in a CO₂ incubator. Cells were washed with PBS and fixed with 2% PFA. Nuclei were stained with DAPI. Tissues and cells were visualized via confocal laser scanning microscopy (Nikon, Melville, N.Y.).

Histological analysis. For hematoxylin and eosin (H&E) staining, tissues were fixed with buffered 10% formalin solution (SF93-20; Fisher Scientific, Fair Lawn, N.J.) overnight at 4° C. Dehydration was achieved by sequential immersion in a graded ethanol series of 70%, 80%, 95%, and 100% ethanol for 40 min each. Tissues were embedded in paraffin and subsequently cut into ultrathin slices (5 μm) using a microtome. Tissue sections were deparaffinized in xylene (Fisher), rehydrated in decreasing concentrations of ethanol in PBS, stained with H&E, and the slides were scanned with an Aperio ScanScope.

In vivo intestinal permeability assay. For in vivo intestinal permeability studies, fluorescein-5-isothiocyanate (FITC)-conjugated dextran (MW 4000; Sigma-Aldrich, St. Louis, Mo.) was administered by oral gavage at a concentration of 60 mg/100 g of body weight. Blood was collected retro-orbitally five hours later and serum was harvested. Fluorescence intensity was determined with a fluorescence spectrophotometer (BioTek) at emission and excitation wavelengths of 485 nm and 528 nm, respectively. FITC concentration was measured from standard curves generated by serial dilution of FITC-dextran.

Cytokines production in plasma & skin tissues. To investigate effects of GDNP on the regulation of cytokine production in peripheral blood and skin tissues, peripheral blood and skin tissues from belly surrounding area was collected from HFD mice treated with PBS or GDNP for 12 months and plasma was extracted. Cytokines were analyzed with a Proteome Profiler Mouse XL Cytokine Array Kit (R&D Systems, ARY028) as per the manufacturer's instructions. Quantification of the spot intensity in the arrays was conducted with background subtraction using HLlmage++ (Western Vision Software).

Enzyme-linked immunosorbent assay (ELISA). Tumor necrosis factor (TNF)-α, interleukin (IL)-1β, IL-6 and IL-10 levels in plasma were quantified using an ELISA method. ELISA reagents were purchased from eBioscience and assays were performed in accordance with the manufacturer's instructions. Briefly, a microtiter plate was coated with anti-mouse TNF-α, IL-1β, IL-6 and IL-10 antibody at 1:200 overnight at 4° C. Excess binding sites were blocked with 100 μl/well of blocking solution (PBS containing 0.5% BSA) at room temperature for 1 hour. After washing three times with PBS containing 0.05% Tween 20, sera collected from mice were diluted 2-fold, added in a final volume of 50 μto the plate wells and incubated for 1 hour at 37° C. After 3 washes with PBS, the plate was incubated with 100 μl of HRP-conjugated anti-mouse antibody (Pierce) diluted 1:50,000 in blocking solution for 1 hour at RT. After the final 3 washes with PBS, the reaction was developed for 15 min, blocked with H2504 and optical densities were recorded at 450 nm using a microtiter plate reader (BioTek Synergy HT).

Insulin signaling Array of hepatocytes cultured with Nano-miR375. 0.3×10⁶ FL83B cells were seeded into six well plate containing DMEM/F12 medium supplemented with 10% FBS. After achieving 50-60% confluence, Nanoparticles (2×10⁶ per mL) were added and incubated for 12 h at 37° C. in a 5% CO₂ atmosphere. Cells were washed with PBS and processed for RNA isolation or protein extraction for western blots. 1 μg of total RNA from FL83B cells treated nanoparticles was reverse transcribed using Superscript III reverse transcriptase (Invitrogen). Insulin signaling array (PAMM030ZE) from Qiagen was performed on Applied Biosystems VIIA™ 7 Real-Time PCR System in accordance with manufacture's instructions.

Indole estimation. Indole levels in mice or human feces and plasma were estimated by using QUANTICHROM™ Indole Assay kit (DIND-100) from BioAssay Systems in accordance with manufacturer's instructions. Briefly, 100 μl of standards or samples were placed into separate wells (triplicates) of clear flat bottom 96 well plate. 100 μl of reagent to each well was added. Plate was tapped to mix briefly and thoroughly. Optical density was measured at 565 nm.

Lipids analysis in plasma. Peripheral blood sample of mice were collected into non-heparinized capillary tubes coated with 4% sodium citrate. The levels of cholesterol and triglycerides were determined by a Piccolo lipid panel plus (Abaxis Inc, USA).

LC-MS analysis of plasma and fecal metabolites. Exsosome-free fecal supernatants and plasma samples from lean, PBS and GDNP HFD mice were used for LC-MS analysis. All samples were analyzed on a Thermo Q Exactive HF Hybrid Quadrupole-Orbitrap Mass Spectrometer coupled with a Thermo DIONEX UltiMate 3000 HPLC system (Thermo Fisher Scientific, Waltham, Mass., USA). The UltiMate 3000 HPLC system was equipped with a hydrophilic interaction chromatography (HILIC) column and a reverse phase chromatography (RPC) column. The HILIC column was a SEQUANT® ZIC®-cHILIC HPLC column (150×2.1 mm i.d., 3 μm) purchased from Phenomenex (Torrance, Calif., USA). The RPC column was an ACQUITY UPLC HSS T3 column (150×2.1 mm i.d., 1.8 μm) purchased from Waters (Milford, Mass., USA). The two columns were configured in parallel 2DLC mode. For 2DLC separation, the mobile phase A for RPC was water with 0.1% formic acid and the mobile phase A for HILIC was 10 mM ammonium acetate (pH adjusted to 3.25 with acetate). Both RPC and HILIC used the same mobile phase B, acetonitrile with 0.1% formic acid. The RPC gradient was 0 min, 5% B, hold for 5.0 min; 5.0 min to 6.1 min, 5% B to 15% B; 6.1 min to 10.0 min, 15% B to 60% B, hold for 2.0 min; 12.0 min to 14.0 min, 60% B to 100% B, hold for 13.0 min; 27.0 min to 27.1 min, 100% B to 5% B, hold for 12.9 min. The HILIC gradient was 0 to 5.0 min, 95% B to 35% B, hold for 1.0 min; 6.0 min to 6.1 min, 35% B to 5% B, hold for 16.9 min; 23.0 min to 23.1 min, 5% B to 95% B, hold for 16.9 min. The flow rate was 0.4 mL/min for RPC and 0.3 mL/min for HILIC. The column temperature was 40° C. for both columns. The injection volume was 2 μL. To avoid systemic bias, the samples were analyzed by 2DLC-MS in a random order. All samples were first analyzed by 2DLC-MS in positive mode followed by 2DLC-MS in negative mode to obtain the full MS data of each metabolite. For quality control purposes, a pooled sample was prepared by mixing a small portion of the supernatant from each sample and was analyzed by 2DLC-MS after injection of every six biological samples. The pooled sample was also analyzed by 2DLC-MS/MS in positive mode and negative mode to acquire MS/MS spectra for metabolite identification. For 2DLC-MS data analysis, MetSign software was used for spectrum deconvolution, metabolite identification, cross-sample peak list alignment, normalization, and statistical analysis. To identify metabolites, the 2DLC-MS/MS data of pooled sample were first matched to our in-house MS/MS database that contains the parent ion m/z, MS/MS spectra, and retention time of 187 metabolite standards. The thresholds used for metabolite identification were MS/MS spectral similarity ≥0.4, retention time difference ≤0.15 min, and m/z variation ≤4 ppm. The 2DLC-MS/MS data without a match in the in-house database were then analyzed using

Compound Discoverer software (Thermo Fisher Scientific, Inc., Germany), where the threshold of MS/MS spectra similarity score was set as ≥40 with a maximum score of 100. The remaining peaks that did not have a match were then matched to the metabolites in our in-house MS database using the parent ion m/z and retention time. The thresholds for assignment using the parent ion m/z and retention time were ≤4 ppm and ≤0.15 min, respectively.

Glucose uptake assay. Glucose uptake was performed in accordance with the manufacturer's instructions. Glucose UPTAKE-GLO™ Assay from Promega (J1341) was used. Briefly, 2×10⁴ cells (hepatocytes cell lines) were seeded in complete medium into a 96-well tissue culture plate. When cells achieved 50-60% confluency, H-Exo plus nanoparticles (1×10⁶) and PBS only as control were added and incubated for 16 h at 37° C. in a CO₂ incubator. Cells were treated with 1 nM of insulin for an additional 1 h. Medium was removed and cells were washed twice with PBS. 50 μl of 2-deoxyglucose (DG, 1 mM per well) was added and incubated for 1 h at RT. 25 μl of stop buffer was added and mixed briefly, and then 25 μl of neutralization buffer was added and shaken briefly. 100 μl of 2DG6P detection reagent was added and the mixture shaken for 3 h at RT. Luminescence was recorded with 135 gain efficiency using a SYNERGY H1 (BioTek) luminometer.

Statistical analysis. Unless otherwise indicated, GraphPad Prism 7.0 (GraphPad software) were used for data analysis. The data are presented as values with standard deviation (mean±SD). The significance of differences in mean values between two groups was analyzed using Student's t-test (one tailed). In case of more than two groups, differences between individual groups were analyzed via one-way (Bonferroni multiple comparison) or two-way ANOVA. Pearson correlation coefficient test was used for two variables such as miR-375 & indole etc. Differences were considered significant when the P-value was less than 0.05. P values >0.05 were considered not significant (NS). * <0.05, ** <0.01, *** <0.001, ****<0.0001.

### Example 9

### Ginger-Derived Nanoparticles (GDNP) Prevent High-Fat Diet-Mediated Inhibition of Foxa2 Expression in the Intestine

Ginger extract administration can prevent HFD-induced obesity and fructose overconsumption-induced insulin resistance in rats. Foxa2 is a downstream target of insulin signaling. Whether GDNP affects the expression of Foxa2 in intestinal epithelial cell was investigated.

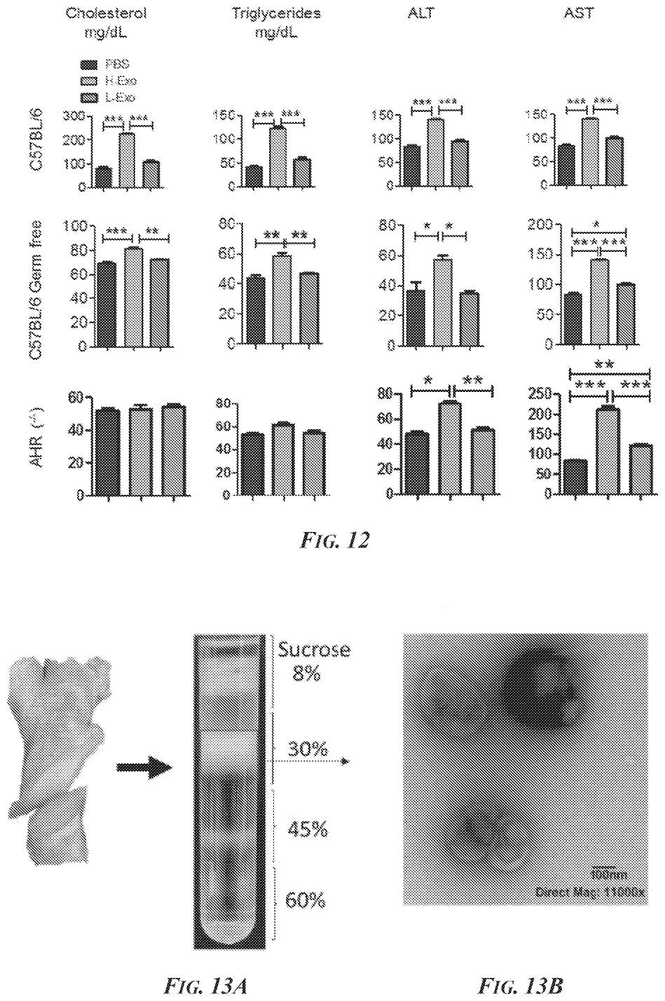

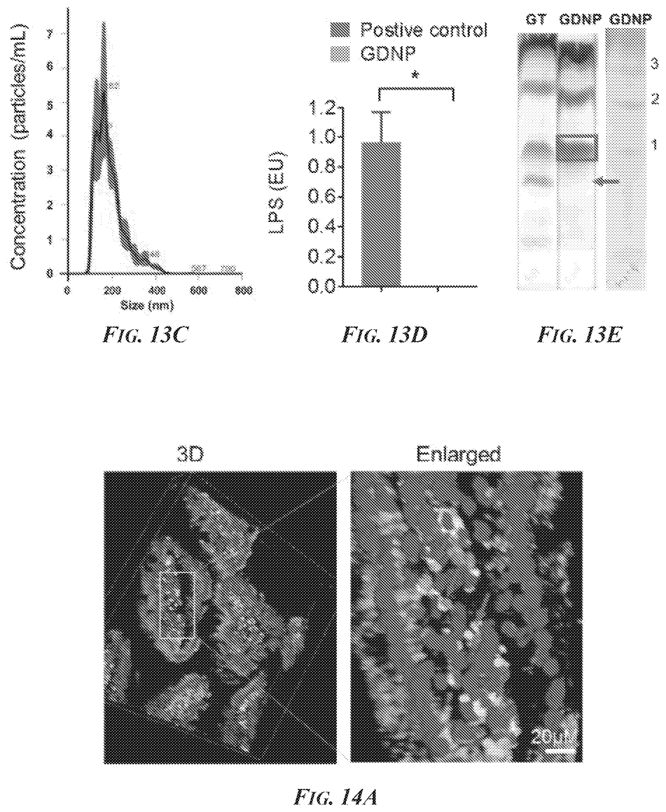

First, GDNP were isolated through differential centrifugation. Sucrose gradient-purified (FIG. 13A) ginger particles from the centrifuged pellet (10,000 g) were characterized by electron microscopy (FIG. 13B) and Nanoparticle Tracking Analysis. The GDNP had a mean size of 162±52 nm (FIG. 13C) with a yield (1×10¹² GDNPs/g ginger tissue). The GDNP had no detectable levels of bacterial LPS (FIG. 13D).

Thin-layer chromatography (TLC) revealed that some lipids in GDNP were enriched (FIG. 13E; box) while others are absent (indicated by arrow) when compared to the lipids extracted from whole ginger root (FIG. 13E). Quadrupole mass spectrometry (MS) analysis of the GDNP lipid profile revealed that phosphatidic acid (PA) represented more than 38.5% of the lipid content, followed by 32.7% digalactosyldiacylglycerol and 21.3% monogalactosyldiacylglycerol (MGDG). Other lipids present in minor concentrations were PI (2.9%), PC (0.02%), PG (0.6%), and LysoPG (3.6%). Further MS analysis of the TLC extracted lipid band (LB1) revealed that >74% of PA present were in LB1 followed by LysoPG (12.4%), DGDG (6.2%), PI (3.7%), and MGDG (2.2%).

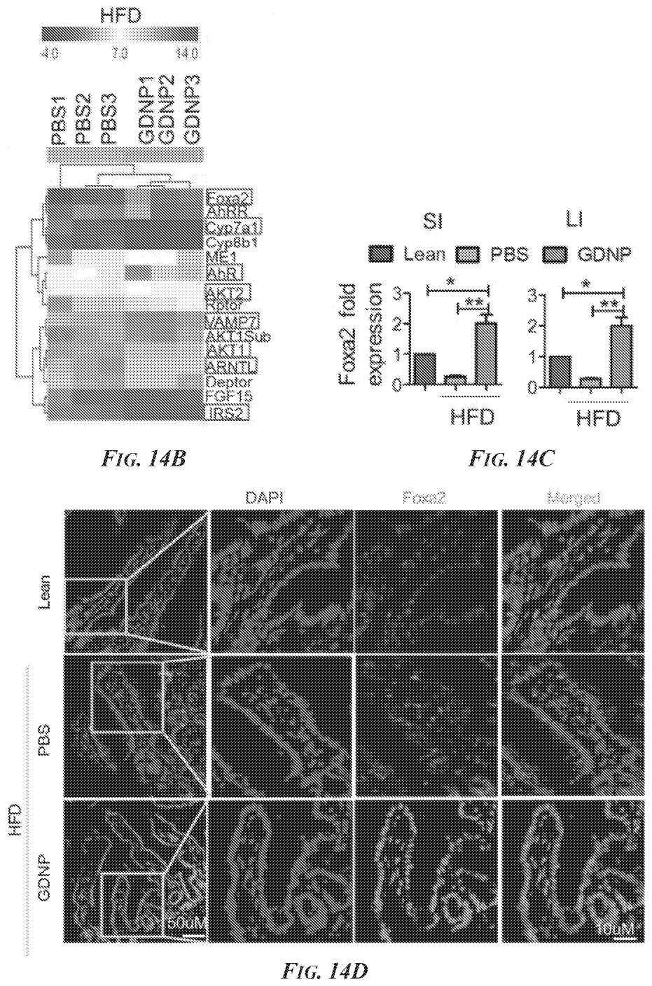

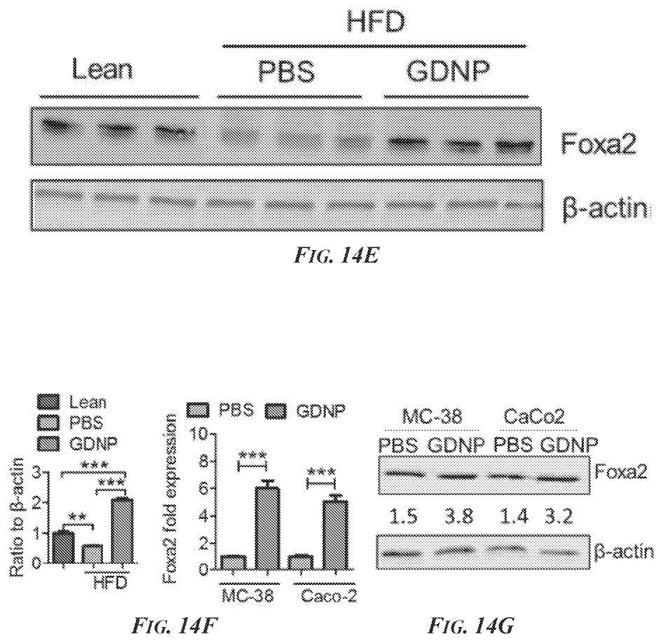

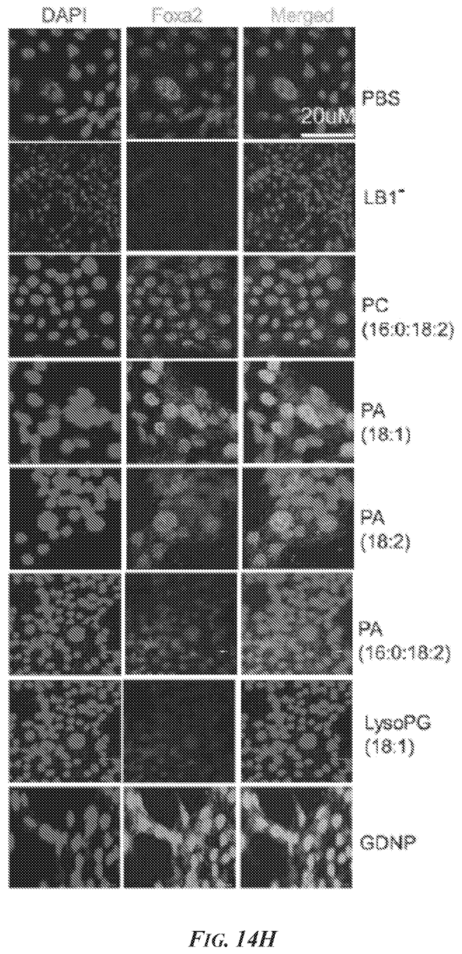

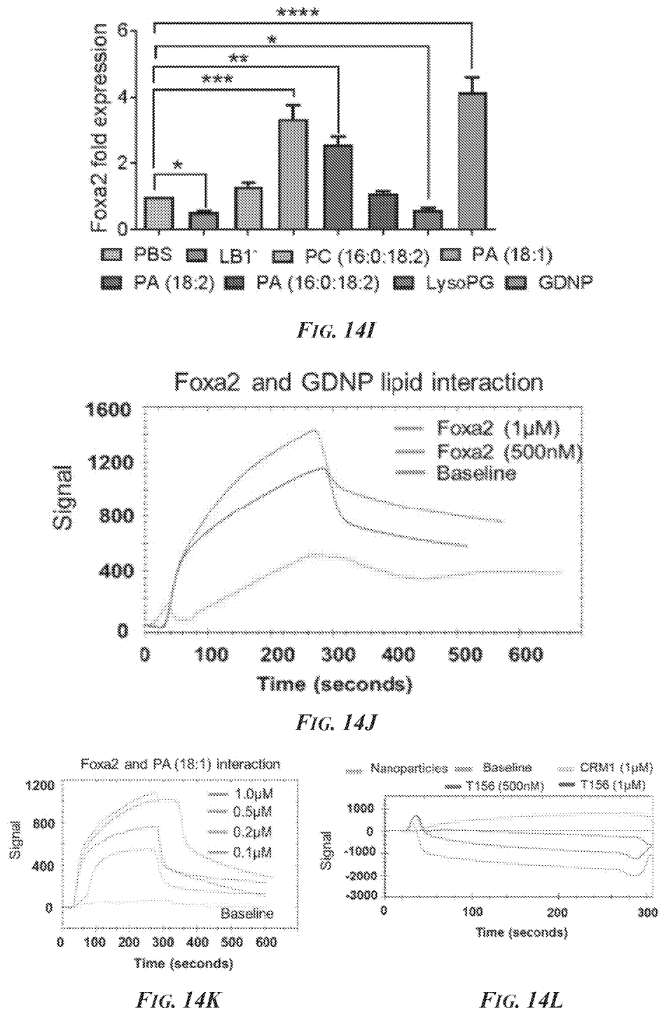

Next, the biological effects of orally administered GDNP were determined. To do so, GDNP were labeled with two different fluorescent dyes (DIR for live imaging and PKH-26 for confocal based analysis), and were then gavaging-given to mice. Live imaging of the mice suggested that GDNP were still present in the intestine at 6 hours. Confocal image analysis of the intestinal tissues suggested that GDNP is taken up by gut epithelial (PKH26⁺A33⁺) cells (FIG. 14A).

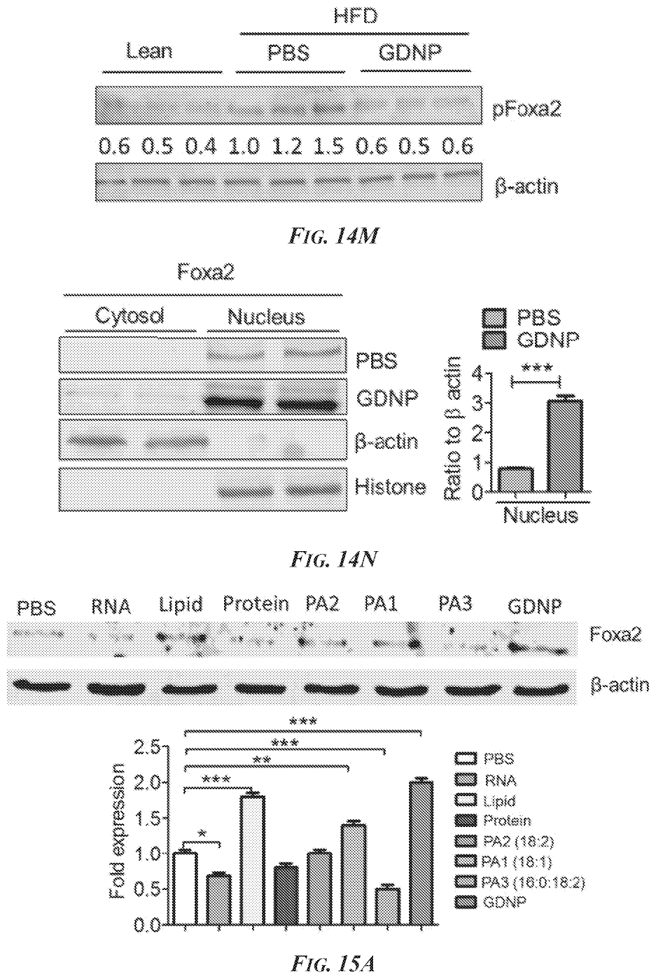

The effects of GDNP uptake on Foxa2 expression were investigated. Affymetrix array analysis of intestinal tissue, followed by confirmation with qPCR, revealed that GDNP treatment induced the expression of Foxa2 and several other genes involved in glucose metabolism and insulin signaling (indicated with boxes, FIG. 14B). The qPCR results suggested that a 12-month HFD feeding led to a decrease in the expression of Foxa2, and GDNP treatment of 12-month HFD-fed mice caused a two-fold increase in Foxa2 mRNA levels in the small and large intestinal tissues relative to lean mice (FIG. 14C). Consistent with the qPCR results, confocal images (FIG. 14D) and western blot analysis of small intestinal tissue (FIGS. 14E and 14F) also suggested an increase in total Foxa2 protein in HFD mice treated with GDNP. Moreover, when mouse (MC-38) and human (Caco2) colon cells were cultured with GDNP (12 hours), the Foxa2 mRNA and protein levels were upregulated (FIG. 14G). Although the GDNP contained proteins and RNAs as well, the effects of GDNP and nanoparticles made from total lipids extracted from GDNP on induction of Foxa2 displayed no differences (FIG. 15A), So, in this study, only GDNP lipids were further studied relative to a Foxa2 mediated insulin response. Taken together, these results suggested that orally administered GDNP can prevent HFD-induced decreases in Foxa2 expression in the intestine.

### Example 10

### Phosphatidic Acid (PA) from GDNP Induced Foxa2 Expression in Intestinal Epithelial Cells

Since PA was the most enriched component of GDNP, whether PA in particular might be responsible for the observed GDNP-induced upregulation of Foxa2 expression in gut epithelial cells was tested. Total lipids were extracted from GDNP and separated by TLC (FIG. 13A), and each lipid band was excised and reconstituted as lipid nanoparticles. Lipid nanoparticles from LB1 extracted from TLC plated total GDNP lipid increased Foxa2 expression (˜4-fold) in MC-38 cells compared to PBS-treated cells (FIG. 15B). Lipid nanoparticles made from total GDNP lipid with LB1 depleted with techniques previously described abolished the induction of Foxa2 expression in these cells, suggesting that LB1 was responsible for the upregulation of Foxa2 (FIG. 14H and 14I; panel LB⁻).

To determine the specific role of PA in the upregulation of Foxa2 expression in intestinal epithelial cells, nanoparticles were generated from commercially available PA lipids. Nanoparticles generated from LysoPG (18:1) and PC (16:0:18:2) were used as controls. Treating MC-38 cells with these lipid nanoparticles indicated that PA (18:1) and (18:2) significantly induced Foxa2 expression, whereas PA (16:0:18:2) did not affect Foxa2 (FIGS. 14H and 14I). Moreover, LysoPG nanoparticles downregulated both Foxa2 protein and mRNA expression (FIGS. 14H and 141). Collectively, these results confirmed that GDNP's PA was largely responsible for the GDNP-induced upregulation of Foxa2 expression in intestinal epithelial cells.

### Example 11

### PA of GDNP Prevented Phosphorylation of Foxa2 by Inhibiting Akt-1 Activation

Whether PA of GDNP not only induced the expression of Foxa2 in intestinal cells but also enhanced the activity of Foxa2 was tested. The potential interaction of GDNP lipids, PA in particular, with Foxa2 was investigated using surface plasmon resonance (SPR), which is an optical technique utilized for detecting molecular interactions. GDNP total lipids were coated on the LIP-1 sensor chip containing a covalently attached lipophilic group to determine whether GDNP lipids interact with recombinant Foxa2 protein. A SPR sensogram peak was identified indicating that GDNP total lipids showed a strong interaction with recombinant Foxa2 protein (FIG. 14J). Furthermore, lipid nanoparticles made from commercially available PA (18:1) were coated onto the LIP-1 sensor, and recombinant Foxa2 was run over the sensor. Consistent with the GDNP total lipid results, Foxa2 recombinant protein also showed a strong interaction with PA (18:1) nanoparticles, and the strength of this interaction was found to be Foxa2 protein concentration-dependent (FIG. 14K).

The PA-binding site of Foxa2 was determined. It has been suggested that phosphorylation of Foxa2 at T156 results in its translocation from the nucleus to the cytoplasm, which in turn leads to its inactivation. Another signal sequence for nuclear exclusion, called CRM1, has also been reported. T156 and CRM1 protein peptides were designed and were run over the PA nanoparticles coated on the LIP-1 sensor. SPR sensograms from the testing of these peptides suggest that the peptide T156 interacts with PA, whereas the CRM1 peptide did not show any notable interactions (FIG. 14L). Altogether, these results confirmed that PA from GDNP binds to Foxa2, potentially at T156.

Phosphorylated Foxa2 (pFoxa2) is translocated from the nucleus to the cytoplasm during insulin exposure (hyperinsulinemia) and results in the inactivation of Foxa2, and it remains permanently inactive in conditions characterized by insulin resistance, such as T2D. Thus, whether GDNP regulates the insulin-mediated phosphorylation of Foxa2 was tested. Relative to lean mice, PBS-treated HFD-fed mice had significantly elevated levels of pFoxa2 in the small intestine tissue. Treatment of HFD-fed mice with GDNP, however, led to a reduction in small intestine pFoxa2 leveled relative to the PBS-treated animals (FIG. 14M), suggesting that GDNP treatment inhibits the phosphorylation of Foxa2. This in vivo result was further confirmed in MC-38 cells, since cells grown in the presence of insulin (50 nM) showed high levels of cytoplasmic pFoxa2 relative to control cells, but co-treatment with insulin and GDNP resulted in reduced levels of pFoxa2 relative to treatment with insulin alone.

Previous studies have shown that phosphorylation of Foxa2 is mediated by pAkt-1, which itself is phosphorylated by mTOR. Whether GDNP treatment inhibited the expression of pAkt-1 and mTOR was thus examined. Indeed, pAKT-1 expression was also reduced in MC-38 cells grown in the presence of insulin (50 nM) relative to controls (p<0.01); co-culture with insulin and GDNP (12 hours) reduced the expression of pAKT-1 (p<0.001). Moreover, the levels of mTOR (p<0.01) and pFoxa2 (p<0.001), but not the pAKT-2 levels, were significantly decreased in GDNP-treated MC-38 cells compared to PBS-treated cells grown in the presence of insulin for 12 hours. Finally, GDNP-treated MC-38 cells showed increased expression of Foxa2 in the nucleus compared to PBS-treated cells (FIG. 14N). Collectively, these results suggested that GDNP treatment blocks insulin-mediated phosphorylation of Foxa2 via the inhibition of pAkt-1.

### Example 12

### Foxa2 Induces miR-375 Expression

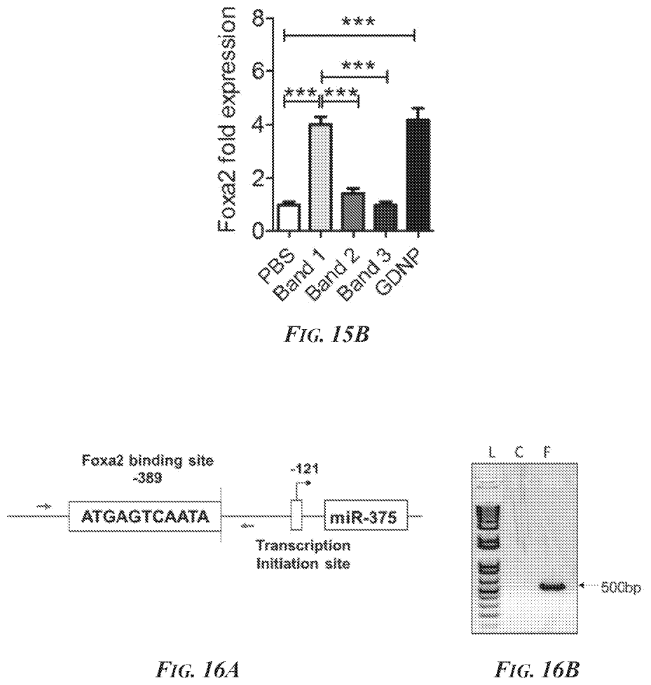

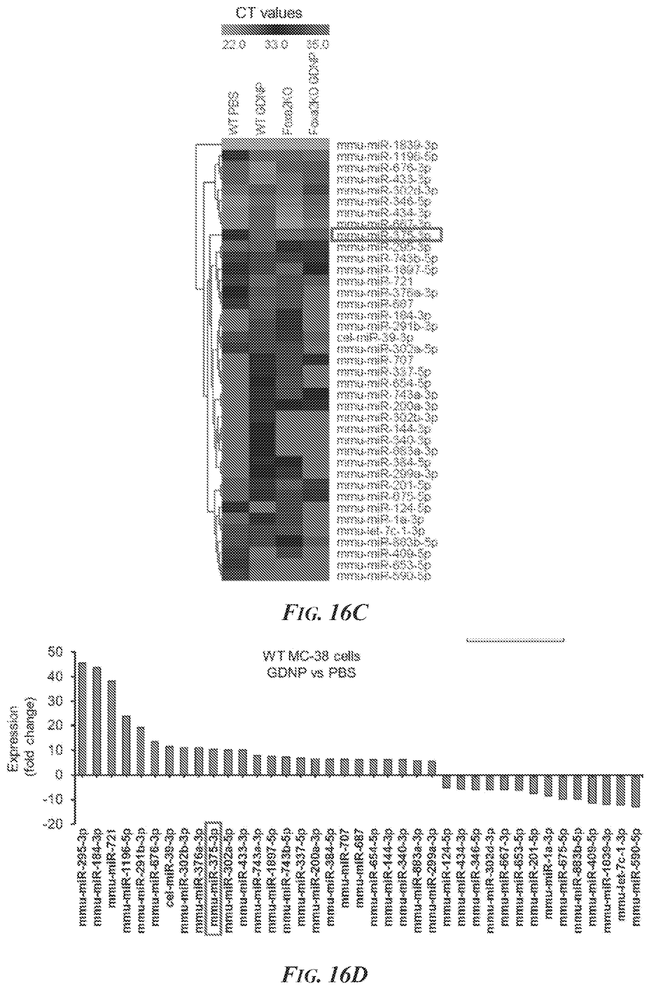

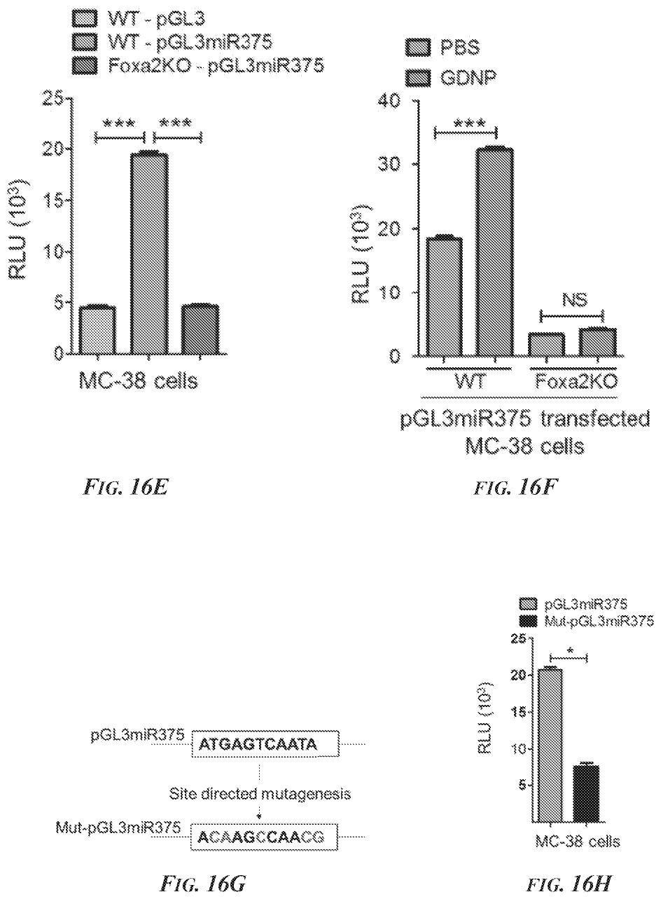

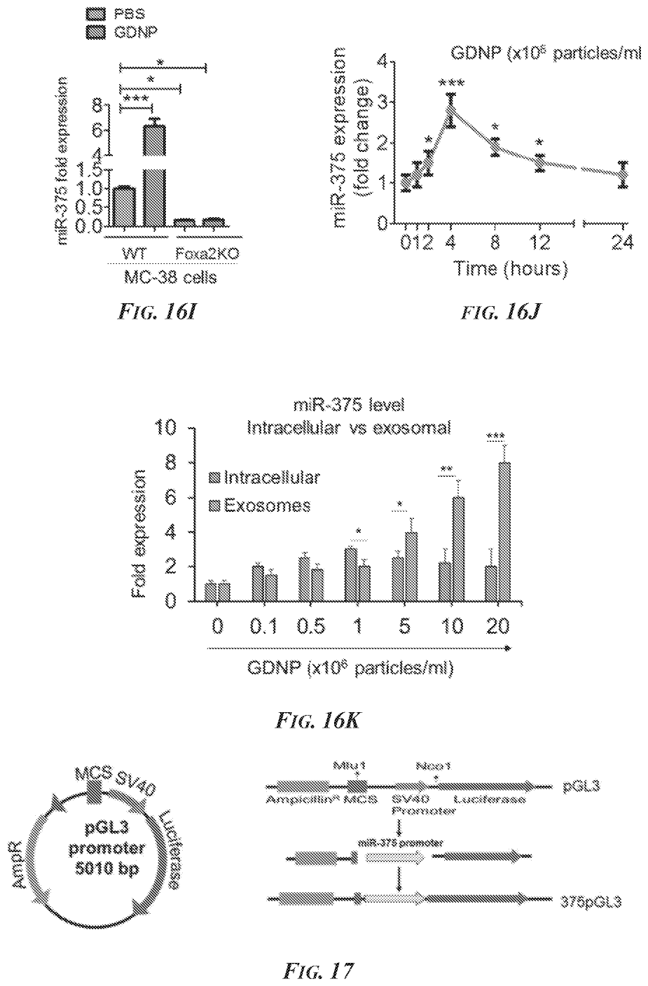

miRNAs regulate multiple pathways including insulin signaling and lipid metabolism, and have important roles in the development of obesity and T2D. Foxa2 regulates the expression of miRNAs that may modulate T2D disease risk. Genetic deletion of miR-375 results in a severely diabetic state. Tfscan (http://www.bioinformatics.nl/cgi-bin/emboss/tfscan) was used to predict Foxa2 binding sites in the mouse genome. Prediction analysis revealed a potential binding site for Foxa2 in the miR-375 upstream sequence (FIG. 16A), and a chromatin immunoprecipitation (ChIP) assay for probing Foxa2-DNA interactions further suggested that Foxa2 binds to the miR-375 promoter (FIG. 16B). To confirm these results, a Foxa2 knockout lentivirus (lentivirus particles generated with a Foxa2 CRISPR/Cas9 plasmid) was used to generate Foxa2 knockout (Foxa2KO) MC-38 cells. A microRNA array of wild-type (WT) MC-38 cells showed a more than 10-fold increase in the expression of miR-375 with GDNP treatment compared to PBS treatment. This GDNP-induced upregulation of miR-375 was greatly reduced in Foxa2KO MC-38 cells (FIGS. 16C and 16D).

The microarray data was further confirmed by measuring pGL3miR375 luciferase activity assay. The miR-375 promoter was cloned into the pGL3-promoter vector (using the cloning strategy illustrated in FIG. 17), and the resulting pGL3miR375 construct was transfected into wild-type (WT-pGL3miR375) and Foxa2 knockout (Foxa2KO-pGL3miR375) MC-38 cells. An approximately four-fold increase in normalized luciferase activity (RLU) was observed in WT-pGL3miR375 cells compared to vehicle (WT-pGL3) and Foxa2KO cells (FIG. 16E), suggesting that Foxa2 regulated expression of miR-375. GDNP treatment of WT—pGL3miR375 cells but not Foxa2 KO cells resulted in a further 1.5-fold enhancement of luciferase activity due to GDNP treatment (FIG. 16F). Moreover, transfection of MC-38 cells with a construct with mutations in the Foxa2 binding site of miR-375 (Mut-pGL3miR375; FIG. 16G) led to a three-fold decrease in luciferase activity relative to transfection with pGL3miR-375 (FIG. 16H). The data generated from luciferase activity was also consistent with miR-375 expression. miR-375 expression in Foxa2KO cells was substantially reduced compared to that in WT MC-38 cells (FIG. 16I), and was not increased in Foxa2KO cells even after GDNP treatment. By contrast, GDNP treatment of WT MC-38 cells upregulated miR-375 expression in a time-dependent manner (FIG. 16J), and the expression peaked after 4 hours of GDNP treatment and declined thereafter. Collectively, these results confirmed that GDNP-mediated Foxa2 induction was responsible for miR-375 induction.

One of possibilities of causing reduction of intracellular miRNAs could result from sorting intracellular miRNAs into exosomes for intercellular communication. Exosomes were thus harvested from MC-38 cells treated with various concentrations of GDNP for 12 hours and analyzed by qPCR. It was determined that the miR-375 level in exosomes increased with increasing concentrations of GDNP, whereas the intracellular expression of miR-375 peaked at a concentration of 10⁶ GDNPs/mL and subsequently decreased with increasing concentrations of GDNP (FIG. 16K). Collectively, these data indicated that GDNP-stimulated Foxa2-dependent upregulation of miR-375 led to the sorting of miR-375 into exosomes in a GDNP dose- and time-dependent manner.

### Example 13

### miR-375-3p Inhibited the Expression of the Aryl Hydrocarbon Receptor

The cumulative findings disclosed herein suggested that GDNP treatment of HFD mice induced miR-375 expression via Foxa2 activation. Moreover, array data (FIG. 14B) suggested that AhR mRNA levels were downregulated in small intestinal tissues from GDNP-treated HFD mice compared to those treated with PBS. With these findings in mind, the potential connection between the induction of miR-375 expression and the downregulation of applicants hereby reserve was tested.

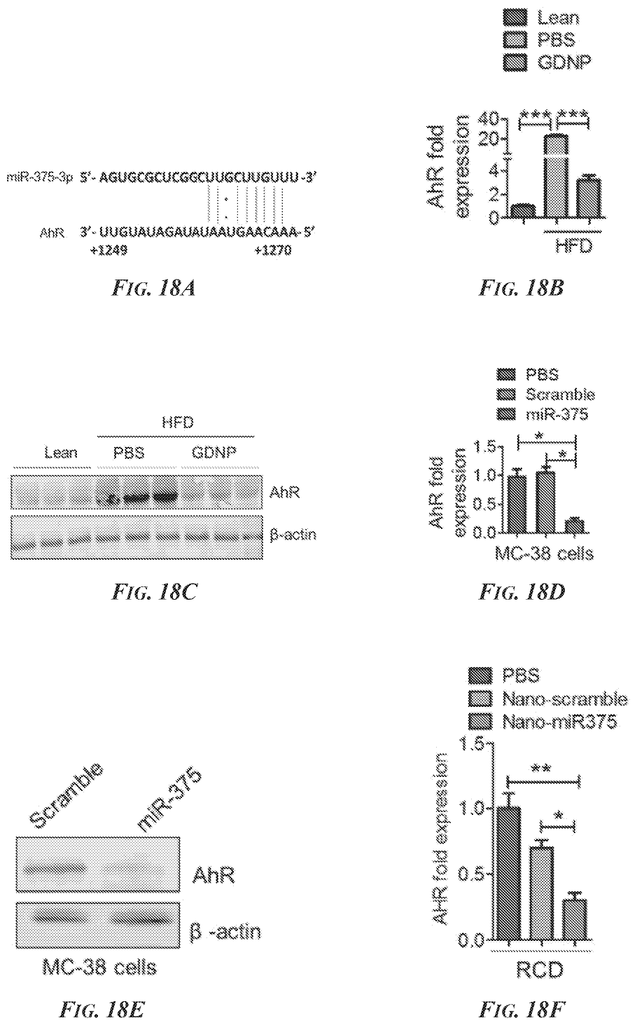

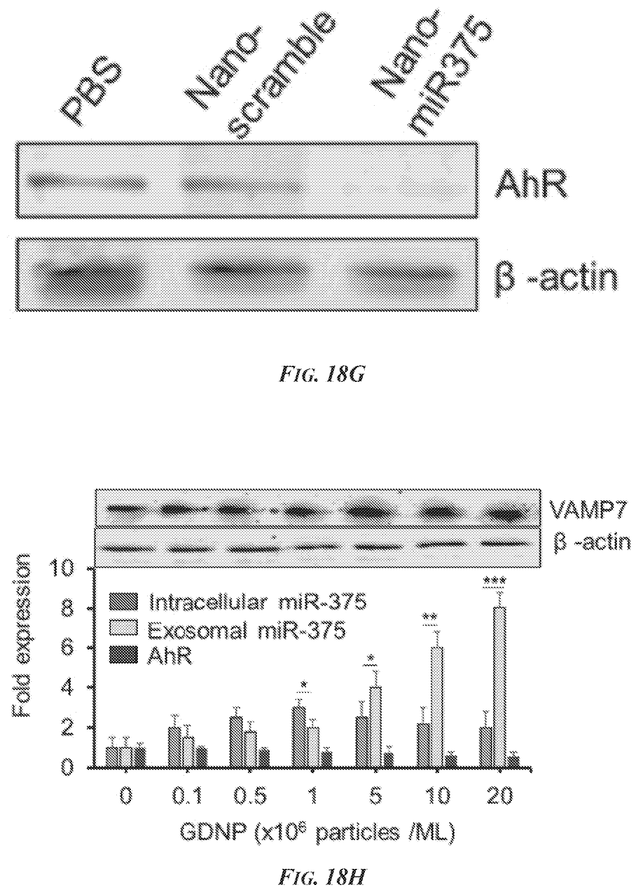

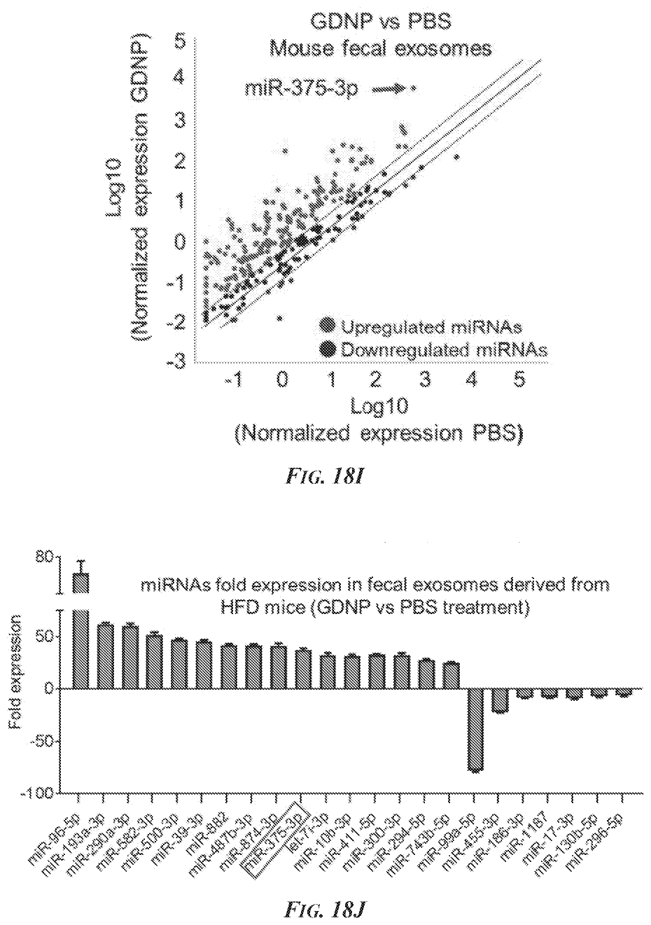

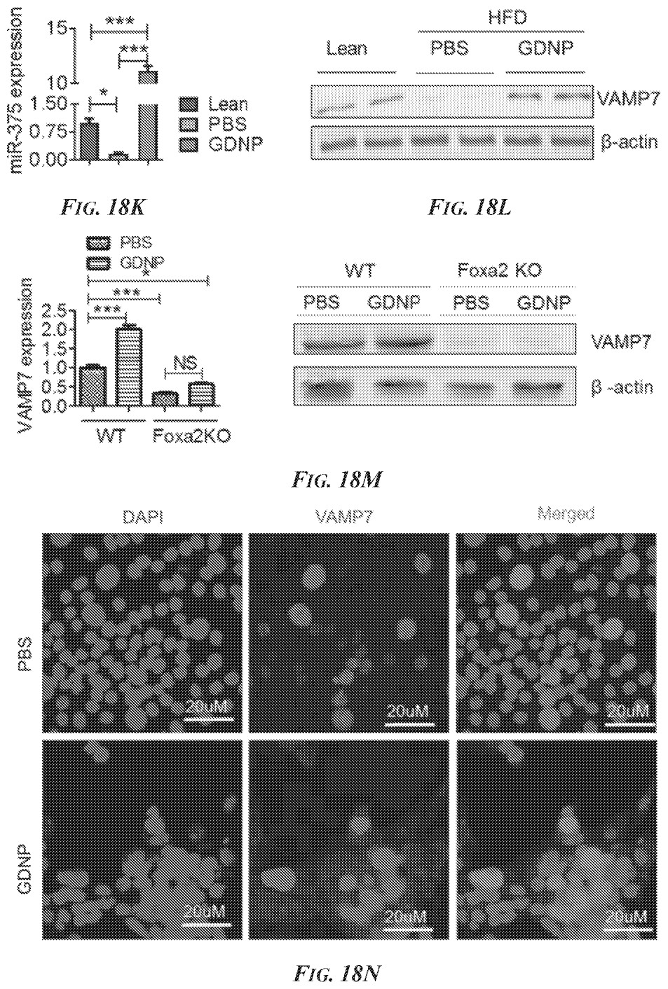

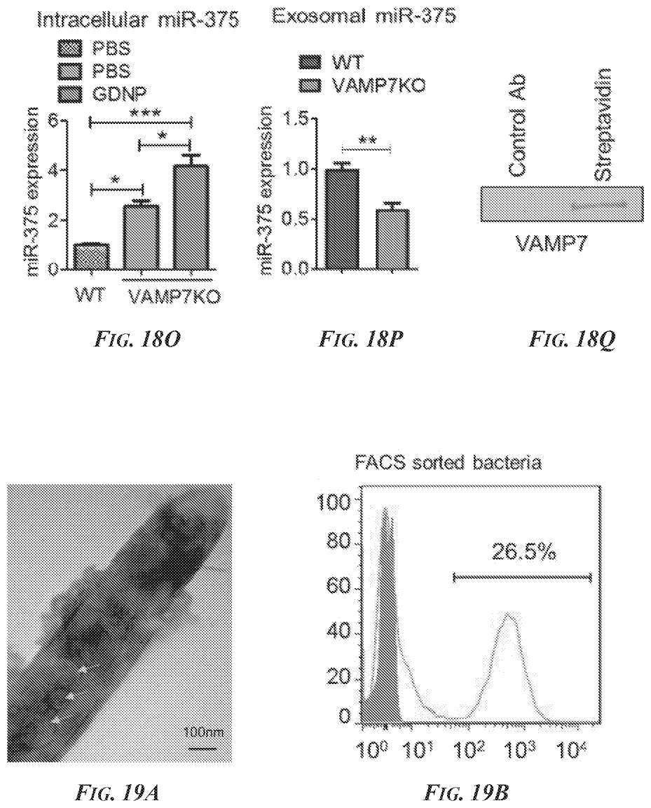

To do so, a BLASTN sequence comparison/search was run against mouse AhR mRNA genomic plus transcript (Mouse G+T) and a potential target site at 3′ UTR of AhR mRNA (FIG. 18A) was identified. Moreover, array and qPCR analyses of small intestinal tissue suggested that GDNP treatment of HFD mice resulted in a ˜7-fold reduction in AhR mRNA levels compared to PBS treatment (FIG. 18B), and western blotting revealed a reduction in AhR protein levels (FIG. 18C). Accordingly, an in vitro assay of transfection of a miR-375 into MC-38 cells led to a reduction in AhR mRNA and protein levels (FIGS. 18D and 18E).

Next, whether miR-375 regulated AhR in vivo was tested. To this end, wild-type C57BL/6 RCD mice were orally administered PBS or nanoparticles made from total lipids extracted from GDNP and packaged with miR-375 (nano-miR375, 20nM) or scrambled RNA (Nano-scramble, 20 nM) daily for 14 days, and the resulting effects on AhR expression were assessed. qPCR and western blot analyses suggested a significant reduction in AhR expression in the small intestine of mice treated with Nano-miR-375 compared to those treated with PBS or Nano-scramble (FIGS. 18F and 18G).

Whether AhR expression was reduced in a GDNP concentration-dependent manner in MC-38 cells was also tested. qPCR assays showed that the greatest reduction in AhR mRNA expression was observed at a concentration of 1×10⁶ GDNPs/mL, and intracellular miR-375 expression was found to be the highest at the same concentration (FIG. 18H; bottom panel). However, at with concentrations higher than 1×10⁶ GDNPs/mL, no further reduction in AhR mRNA expression was observed in the MC-38 cells treated, and the levels of intracellular miR-375 began to decrease, whereas levels of exosomal miR-375 began to increase (FIG. 18H, bottom panel). Collectively, these results suggested that GDNP treatment induced the expression of miR-375. The increased expression of miR-375 inhibited AhR expression.

### Example 14

### miR-375 was Sorted into Intestinal Epithelial Cell Exosomes via Foxa2-Mediated Induction of VAMP7, and GDNP-Induced Foxa2 Restored HFD Disrupted Gut AhR Homeostasis

Using an Affymetrix array of the small intestine, it was determined that GDNP-treated HFD-fed mice also showed increased VAMP7 expression in the small intestine relative to PBS treated mice. The induction of VAMP7 led to increasing exosomal miR-375 when MC38 cells were treated with GDNP at concentrations higher than 1×10⁶ GDNPs/mL (FIG. 18H, top panel). It has been suggested that VAMP7 is involved in biogenesis of the exosomes. The in vitro data disclosed herein suggested that GDNP treatment resulted in the sorting of miR-375 into exosomes, and led to the investigation of whether the composition of intestinal epithelial cell-derived exosomal miRNAs is regulated by diet.

The miRNA levels in fecal exosomes from HFD-fed mice that had been fed with GDNP- and PBS for 12 months were analyzed. Both exosomes from GDNP- and PBS-treated HFD-fed mice were positive for CD63, CD81, CD9 (exosomal marker) and A33 (intestinal epithelial cell marker) as assessed by western blot. miRNA array and qPCR data (FIG. 18I) revealed that fecal exosomes from HFD mice treated with GDNP (>12 months) contained significantly higher levels of miR-375 compared to those of PBS-treated HFD mice (FIGS. 18J and 18K). Western blots of the intestinal tissue extracts from HFD-fed mice treated with PBS showed substantially reduced VAMP7 expression relative to lean mice, while HFD-fed mice treated with GDNP showed increased expression of VAMP7 compared to lean controls (FIG. 18L).

Whether Foxa2, which was upregulated by GDNP, affected VAMP7 expression was investigated. qPCR and western blot results demonstrated that GDNP treatment of WT MC-38 cells increased the expression of VAMP7 compared to treatment with PBS, while Foxa2KO MC-38 cells showed decreased VAMP7 expression compared to WT cells following both PBS and GDNP treatment (FIG. 18M). Consistent with the western blot results, confocal images of MC-38 cells treated with GDNP showed increased expression of VAMP7 compared to treatment with PBS (FIG. 18N).

Next, VAMP7 knockout (VAMP7KO) MC-38 cells were generated in order to assess the role of VAMP7 in miR-375 sorting. The qPCR analysis of miR-375 expression in VAMP7KO cells and exosomes suggested that miR-375 accumulated in the cells (FIG. 18O) and in turn was reduced two-fold in the exosomes (FIG. 18P). For determination of whether VAMP7 directly interacts with miR-375, MC-38 cells were transfected with biotinylated miR-375 and pulled down with an streptavidin-conjugated antibody. Western blot analysis of the pulldown product with antibodies against VAMP7 indicated that VAMP7 indeed binds to miR-375 (FIG. 18Q). These results suggested that VAMP7 monitored intracellular levels of miR-375 to prevent an uncontrolled reduction of AhR by sorting miR-375 into exosomes.

Accumulated results indicated that GDNP-induced Foxa2 has a role in restoring AhR homeostasis expression disrupted by a HFD. These results include Foxa2 upregulating expression of miR-375 (FIGS. 16C-16E) and VAMP7 (FIG. 18M), and miR-375 inhibiting the expression of AhR (FIGS. 18D-18G). When the concentration of GDNP reached higher than 1×10⁶ particles/ml, miR-375 was sorted out in a VAMP7 dependent manner and no more reduction of AhR took place (FIG. 18H). Collectively, Foxa2 induced VAMP7 for maintaining homeostatic AhR expression by sorting miR-375 into exosomes. The level of reduction of AhR provided a feedback loop signal for VAMP7 initiation of sorting miR-375 out.

### Example 15

### Exosomal miR-375 Bound to the E. coli Tryptophanase (tnaA) Gene and Decreased Indole Production

As disclosed herein, treating mice with GDNP led to the induction of miR-375 expression and the sorting of this miRNA into intestinal epithelial exosomes by VAMP7. It is known that intestinal epithelial cells (IECs) release miRNAs packed in exosomes into the intestinal lumen. IEC exosomal miRNAs in turn influence the composition of gut bacterial populations.

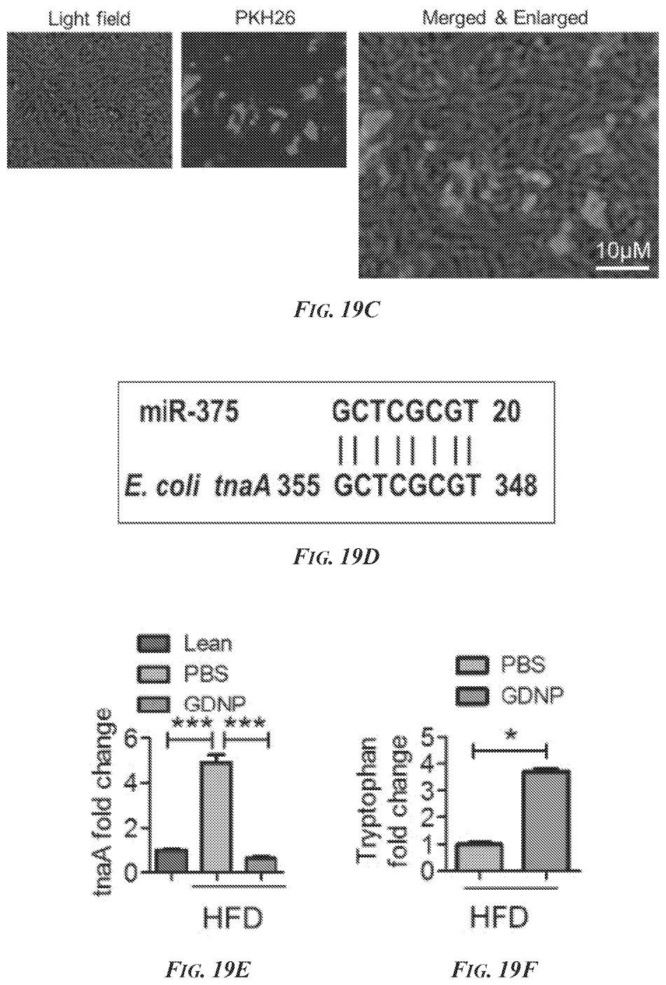

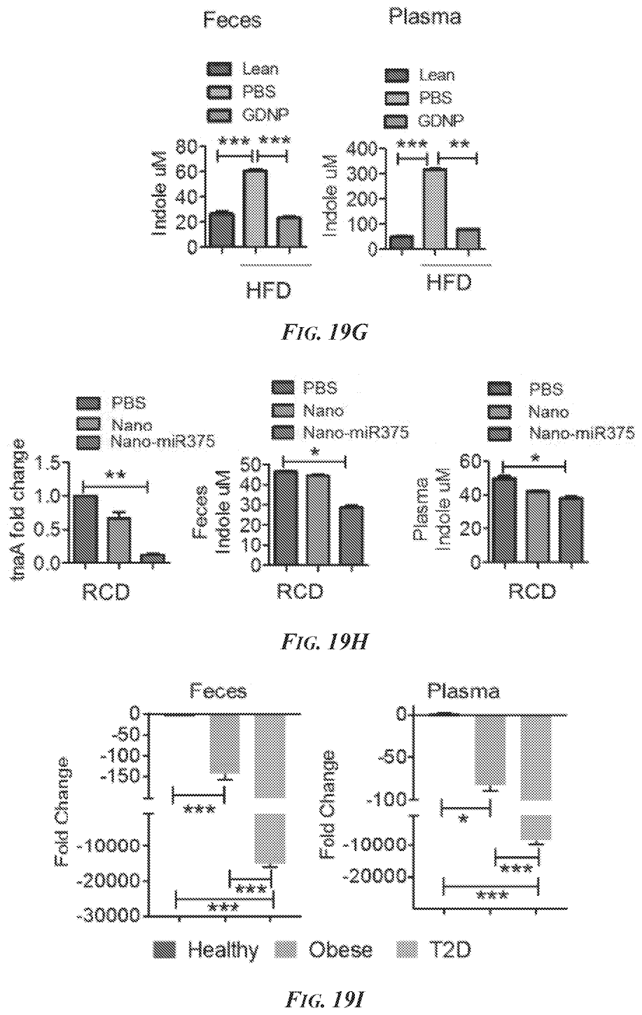

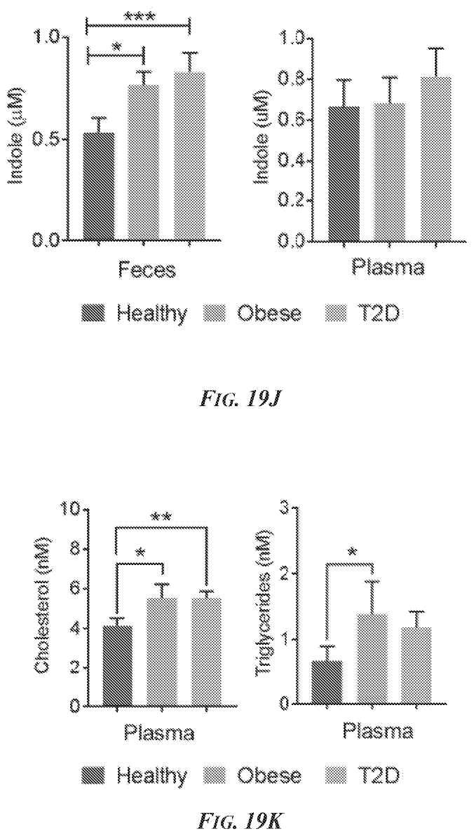

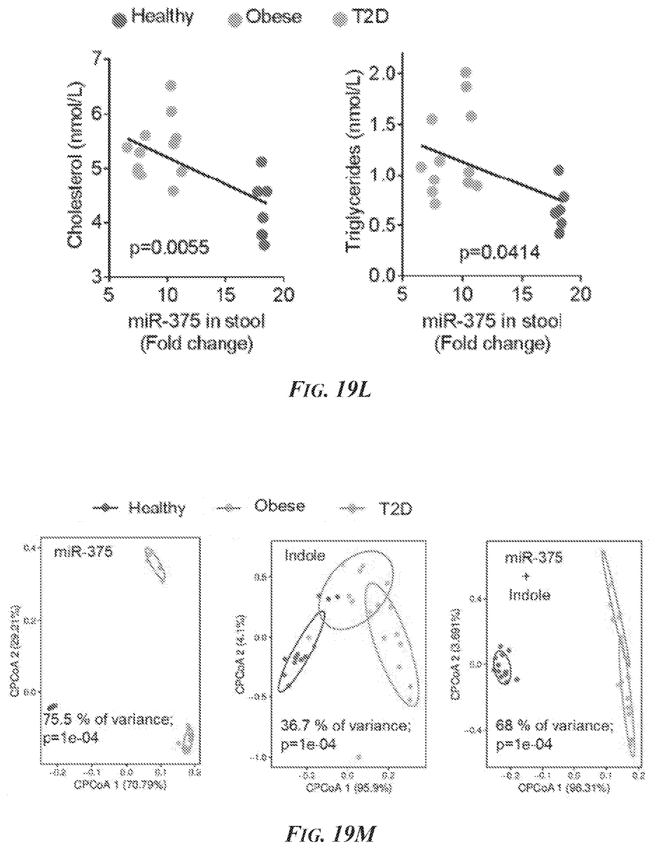

Using electron microscopy, IEC-exosomes were observed to be taken up by gut bacteria (FIG. 19A). To further confirm the gut bacteria uptake of exosomes released by IECs, PKH26-labeled epithelial cell-derived (CD63⁺A33⁺) fecal exosomes were administered orally to wild-type C57BL/6 mice. It was determined that exosomes released into the lumen were indeed taken up by gut microbiota (FIGS. 19B and 19C). Analyses of PKH-26-positive FACS-sorted bacteria suggested that 26.5% of gut bacteria contained PKH-26-labeled fecal exosomes.

As miR-375 is increased in fecal exosomes from GDNP treated HFD-fed mice and intestinal exosomes are taken up by gut bacteria, whether exosomal miRNAs targeted bacterial genes was investigated. A BLASTN search was performed against the E. coli genome with the mmu-miR-375-3p sequence, and a putative binding site for miR-375-3p in the tryptophanase (tnaA) gene of E. coli was identified (FIG. 19D). To determine whether GDNP-mediated induction of miR-375 affects the expression of tnaA mRNA, gut bacteria were harvested from HFD mice treated with PBS or GDNP and qPCR was used to assess the levels of the E. coli tnaA gene. It was found that the gut bacteria from GDNP-treated HFD-fed mice showed approximately a five-fold decrease in tnaA gene expression compared to PBS-treated HFD-fed mice (FIG. 19E).

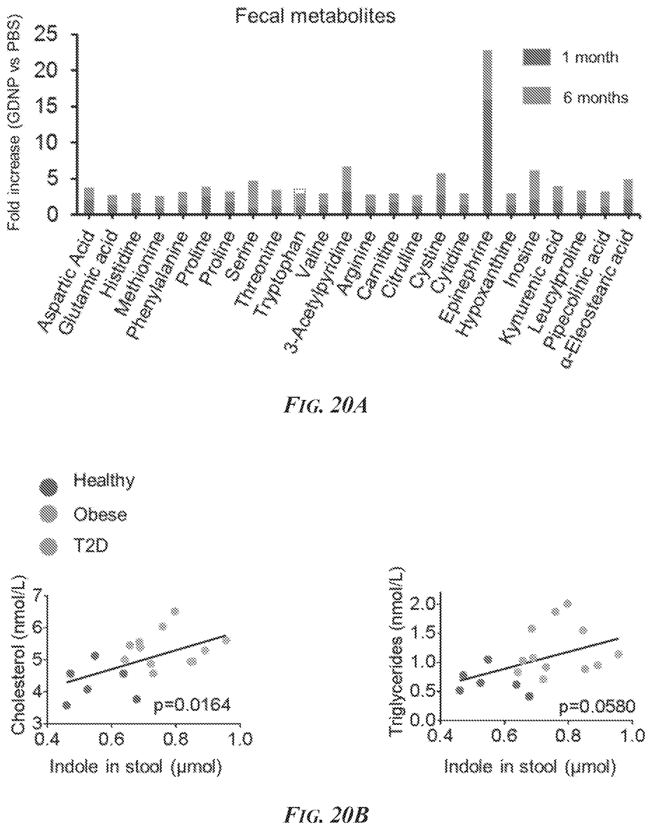

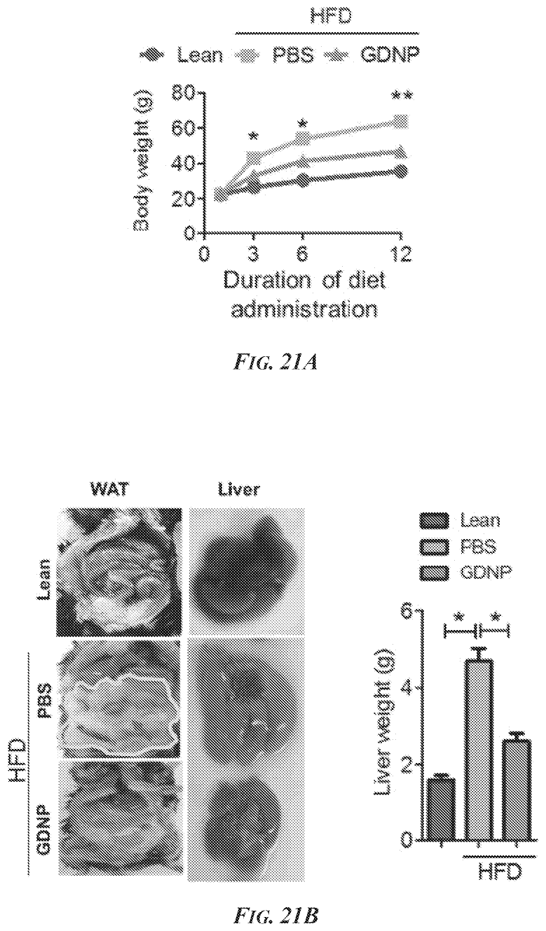

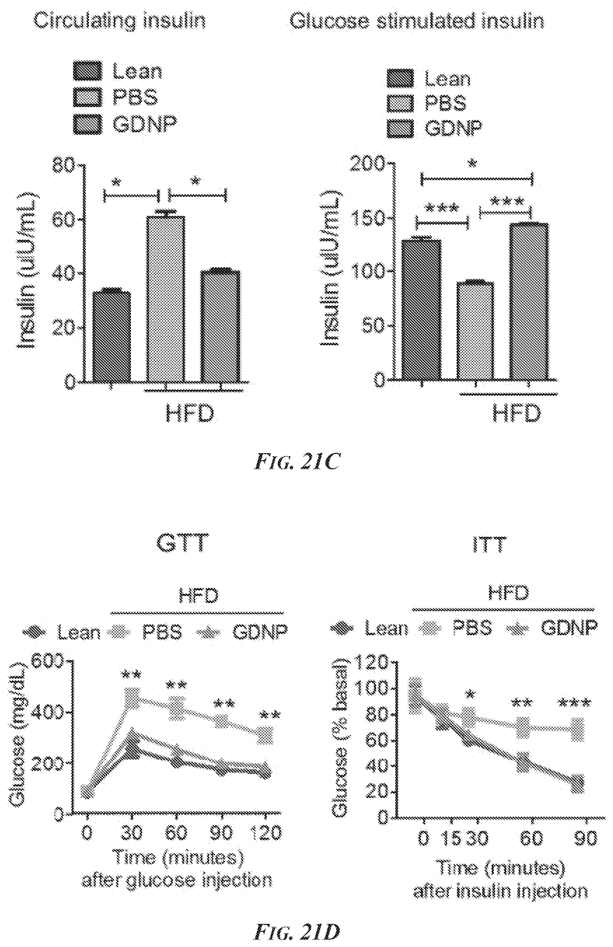

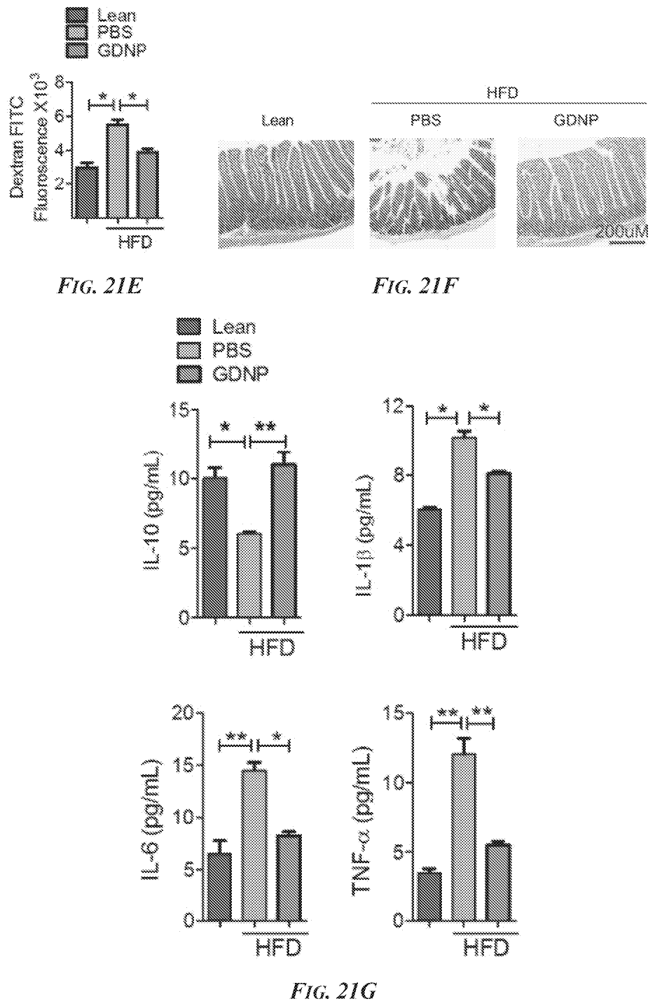

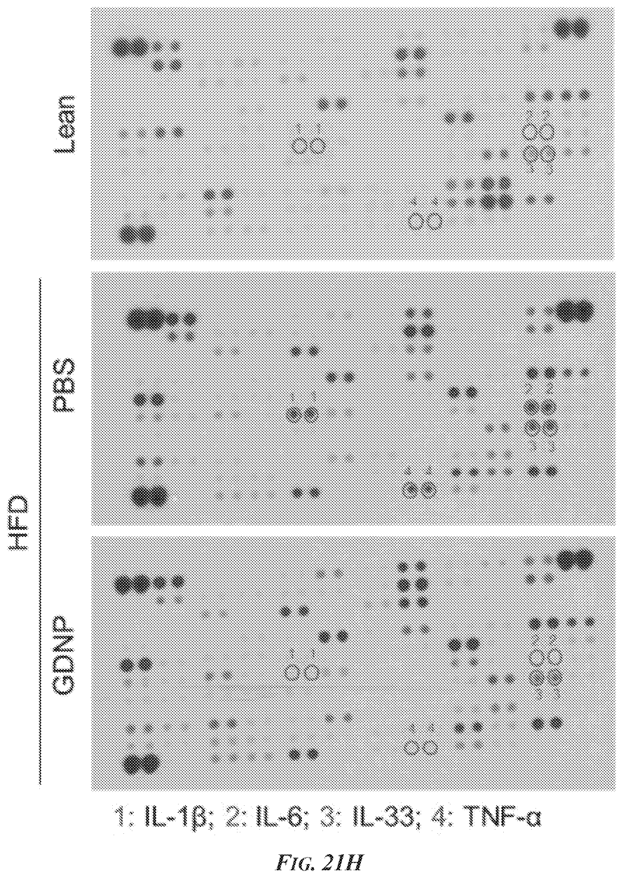

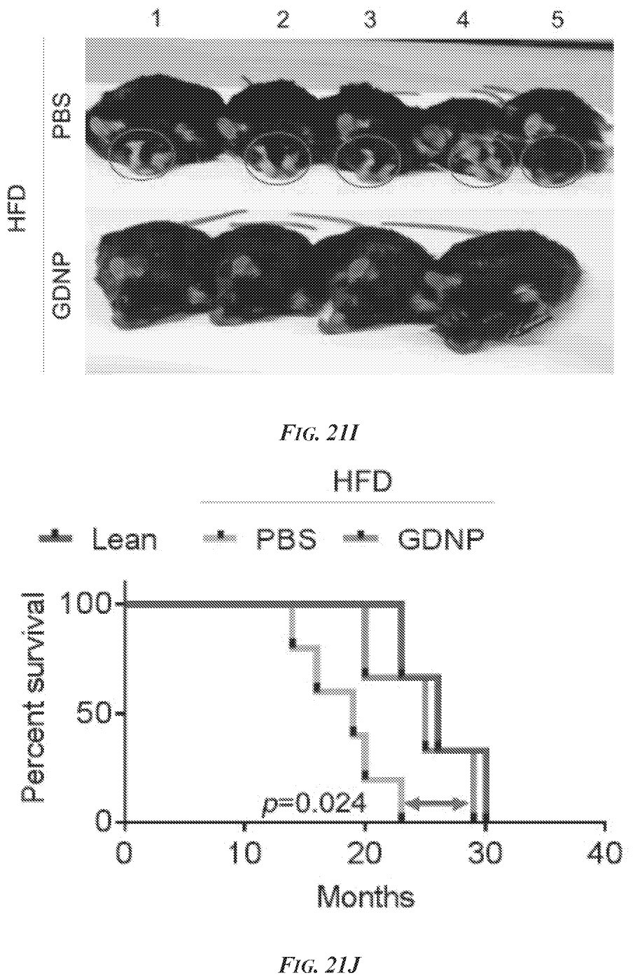

It was reasoned that downregulation of the tryptophanase enzyme may affect tryptophan metabolism and, subsequently alter levels of tryptophan-derived metabolites such as indole. 2D LC-MS/MS analysis of fecal metabolites was performed, and it was determined that tryptophan was not completely metabolized in GDNP-treated HFD-fed mice, as evidenced by elevated levels of un-metabolized tryptophan excreted in their feces compared to PBS-treated animals (FIG. 19F and FIG. 20A). Moreover, the indole levels in the fecal supernatants and plasma from GDNP-treated HFD-fed mice were reduced (>3- and 5-fold, respectively) compared to those in the PBS treated group (FIG. 19G).

Whether the reduction of indole levels in GDNP-treated HFD-fed mice resulted from the increased expression of miR-375 was investigated. GDNP lipid nanoparticles generated with or without miR-375 were orally administered to wild-type C57BL/6 mice daily for 14 days. The qPCR results suggested that mice receiving Nano-miR-375 had an approximately 10-fold reduction in tnaA mRNA levels as well as a reduction in the indole levels in the feces and plasma compared to PBS treated mice (FIG. 19H).

### Example 16

### Human Fecal Exosomal miR-375 was Negatively Correlated with Indole Production

To determine whether the findings generated from obese HFD-fed mice were applicable in patients with obesity-induced T2D, CD63⁺A33⁺ exosomes were isolated from stool samples of healthy and obese individuals and patients with T2D. qPCR analysis of miR-375 levels in these exosomes was then performed. Consistent with the mouse data presented herein, both obese patients and individuals with T2D showed large reductions in miR-375 expression in exosomes from feces and plasma (FIG. 19I) compared to healthy controls. Furthermore, the indole levels in fecal supernatants (FIG. 19J), but not in plasma, were increased in obese patients and patients with T2D compared to healthy individuals. Plasma cholesterol and triglyceride levels were also elevated in obese and T2D individuals relative to healthy controls (FIG. 19K).

Moreover, when linear correlation analysis was performed for miR-375 (feces exosomes) vs. cholesterol and triglyceride levels, the clusters were highly segregated for healthy controls, obese patients and patients with T2D (FIG. 19L). Principal component-based analysis (PCoA), which has been used to extract independent factors from inter-correlated factors, resulted in the classification of subjects into two groups. Subjects with low fecal exosomal miR-375 and a high level of indole were associated with obesity and the T2D group. Collectively, the data supported the hypothesis that miR-375 expression was negatively correlated with fecal indole production (FIG. 19M). These findings suggested that the level of miR-375 in fecal exosomes released by IECs was a critical factor for the inhibition of indole production and could be used as a prognostic biomarker for T2D and obesity.

### Example 17

### Oral Administration GDNP Prevented Glucose Intolerance and Insulin Resistance

Whether oral administration of GDNP prevented HFD-induced glucose intolerance and insulin resistance was tested by administering GDNP in drinking water for 12 months. The data suggested that GDNP treatment of HFD-fed mice prevented the HFD-induced increases in body weight, liver weight, and white adipose tissue, inhibited the development of hyperinsulinemia (FIGS. 21A-21C), and protected against the development of glucose intolerance and insulin resistance (FIG. 21D).

HFD is known to increase gut permeability, therefore, whether gut permeability was altered in HFD-fed mice treated with PBS or GDNP (>12 months) was tested using dextran FITC. The dextran FITC results suggested that mice receiving PBS had elevated plasma levels of dextran FITC compared to GDNP-treated HFD-fed mice and lean mice, suggesting elevated levels of plasma dextran FITC increased gut permeability (FIG. 21E). Moreover, histological (H & E staining) analysis of the small intestine indicated that HFD-fed mice that received GDNP for >12 months showed normal intestinal integrity, similar to that of lean mice, whereas HFD-fed mice that received PBS showed disrupted villi, which was indicative of compromised gut integrity (FIG. 21F).

GDNP treatment also inhibited the HFD-induced increase in pro-inflammatory cytokines including IL-1β, TNF-α, and IL-6, and induced anti-inflammatory cytokine, IL-10 (FIG. 21G). High blood sugar can lead to dehydration, dry skin, and inflammation. Indeed, a cytokine array of skin tissue from GDNP-treated HFD-fed mice revealed that the levels of skin inflammatory cytokines, including IL-33, were significantly downregulated compared to PBS treated mice (FIG. 21H). This reduction of inflammatory cytokines was consistent with decreased immune cell (F4/80 and CD3) infiltration in GDNP treated HFD-fed mice relative to PBS treatment (confirmed by fluorescence immuhistochemistry).

Recently, IL-33 was identified as an inflammatory agent in skin, specifically in dermatitis. GDNP treatment also prevented skin lesions and reduced the appearance of gray fur (FIG. 21I). This low-grade, chronic inflammatory condition is a universal feature of aging and plays a significant role in morbidity and mortality in elderly individuals. It was also found that GDNP-treated HFD-fed mice had a 3-5-month increase in lifespan compared to PBS-treated HFD-fed mice (FIG. 21J). Collectively, these findings suggested that GDNP added to drinking water improved the overall health of HFD-fed mice throughout their lifespan.

### Example 18

### miR-375 Protected Mice from Fecal Exosome-Mediated Insulin Resistance Transfer and Glucose Intolerance

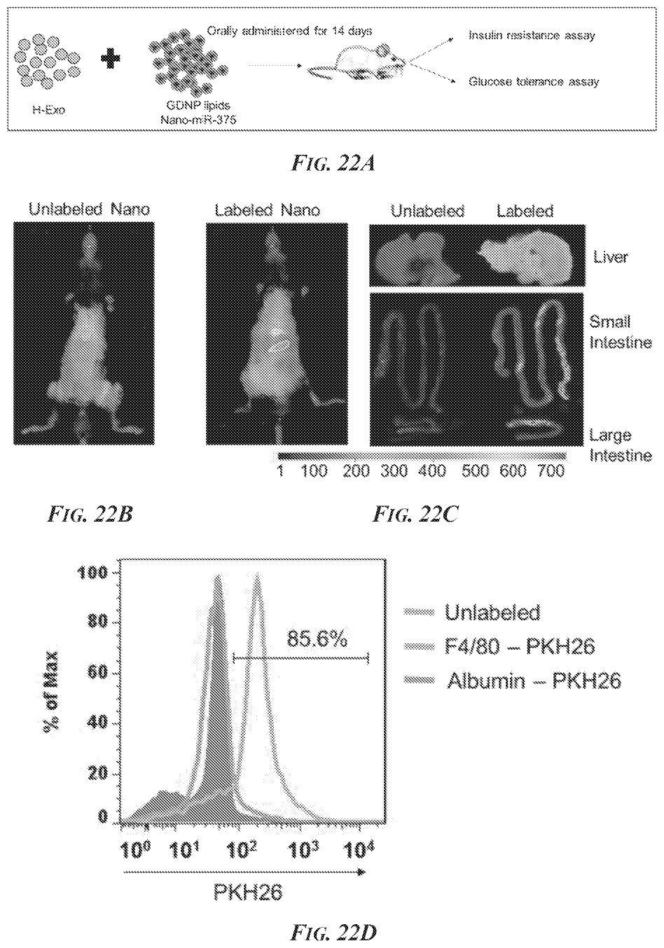

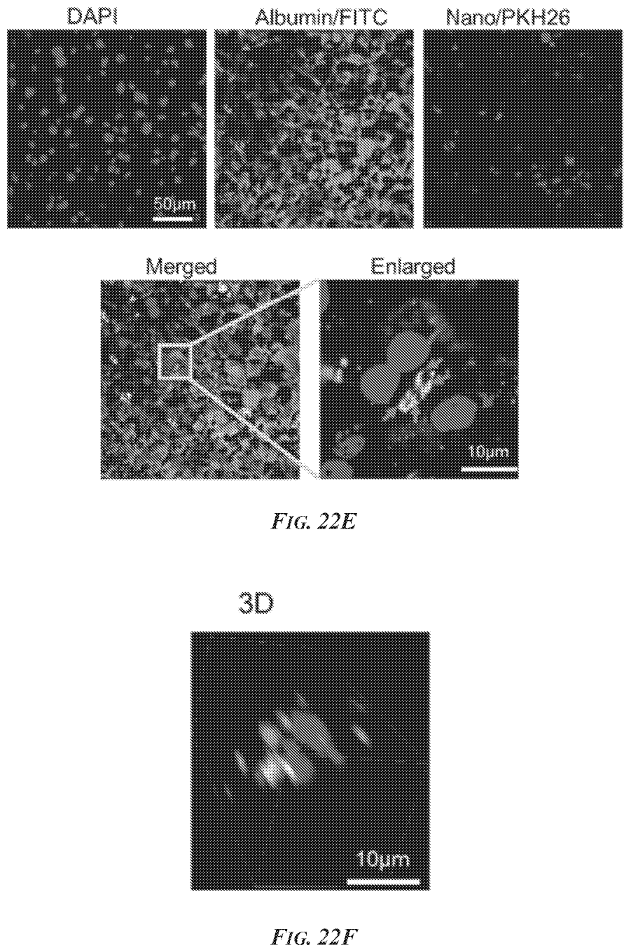

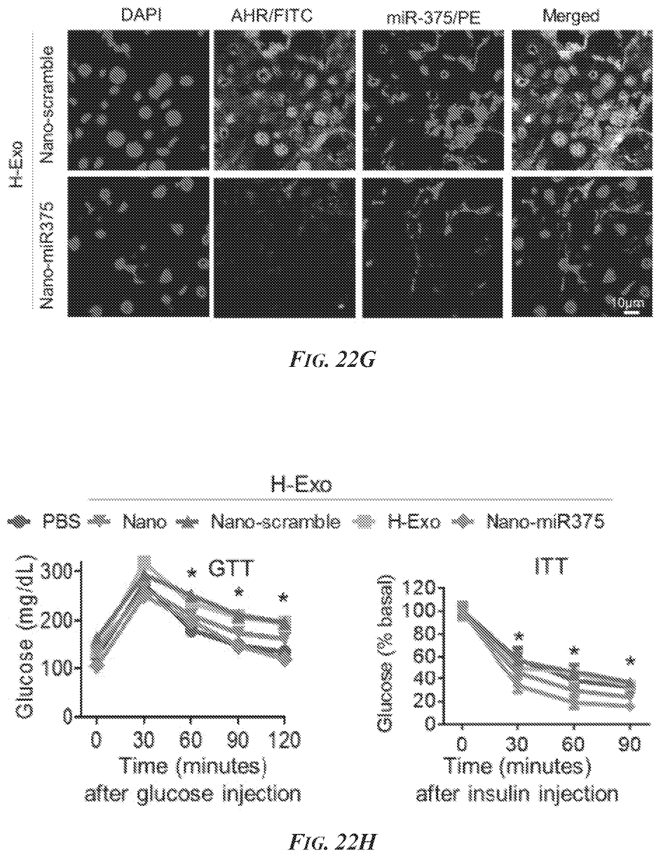

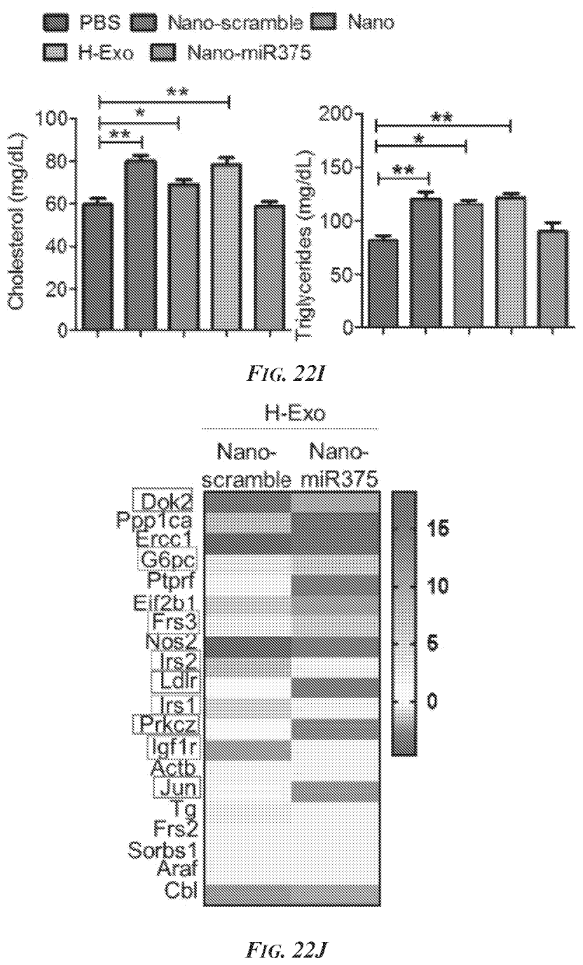

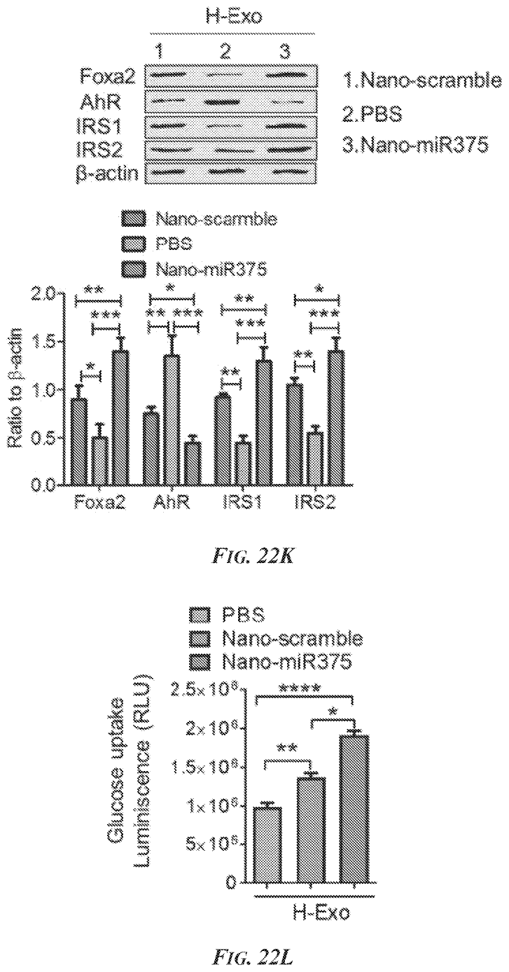

In vivo administration of stool EVs from HFD-fed mice induced insulin resistance and glucose intolerance. Here, whether miR-375 carried by a ginger nano-vector made from total lipids extracted from GDNP was taken up by hepatocytes and prevented stool EVs of obese HFD-fed mice (H-Exo) induced insulin resistance by decreasing AhR-mediated signaling in hepatocytes was tested. miR-375 (Nano-miR375) or scrambled RNA (Nano-scramble) was packaged into ginger nano-vector and orally administered daily for 14 days along with fecal exosomes (CD63⁺A33⁺, H-Exo) isolated from HFD-fed B6 mice for 12-months to lean C57BL/6 mice (FIG. 22A). Live imaging of the mice suggested that PKH26 and DIR dye double-labeled ginger nano-vectors were transported to the liver at 6 h post-administration (FIGS. 22B and 22C).

Further analysis of cellular uptake was explored by flow cytometry and confocal microscopy. We found that ginger nano-vectors were taken up by hepatocytes (albumin⁺) (FIGS. 22D-22F). Functional analyses showed that the group that received H-Exo plus Nano-miR-375 had lower levels of AhR (FIG. 22G) and improved glucose tolerance and insulin sensitivity compared to all other groups including the nano-scramble group, nano vector, and PBS (FIG. 22H). However, mice that received only H-Exo developed glucose intolerance and insulin resistance. Insulin resistance can also alter systemic lipid metabolism, which then leads to the development of dyslipidemia. Nano-miR375 treatment inhibited the development of dyslipidemia, as evidenced by restoring homeostasis of blood cholesterol and triglycerides (FIG. 22I).

Next, the Nano-miR375 treatment effects on expression of hepatic genes that regulates insulin signaling were assessed by insulin signaling PCR array. After a 24 hour culture, primary mouse hepatocytes were co-cultured with H-Exo (100 ng/mL) plus Nano-miR375 or Nano-Scramble-miR (20 nM) or PBS as a control for an additional 24 hours. RNA was extracted from 24 hour-treated hepatocytes and used for insulin signaling PCR array (Qiagen). The array data suggested that the expression of G6Pc, Frs3, IRS2, IRS1, and IGF1R were upregulated (FIG. 22J, light gray boxes), whereas DoK2, Ppp1ca, Ptprf, Ldlr, PrkcZ, and Jun were downregulated after Nano-miR375 treatment compared with hepatocytes treated with Nano-Scramble (FIG. 22J, dark gray boxes). Protein levels were confirmed by western blot analyses (FIG. 22K). These PBS-Exo treated hepatocytes also showed inhibited glucose uptake (FIG. 22L). However, these inhibitory effects were dampened in GDNP exosome-treated hepatocytes.

Moreover, plasma levels of free amino acids associated with T2D were elevated in HFD-fed mice, whereas GDNP treatment reduced plasma levels of free amino acids. Free amino acids which have been known to be beneficial in T2D were upregulated in GDNP-treated HFD mice. Collectively, these data suggested that oral delivery of miR375 packed in GDNP-based nanovectors prevented glucose intolerance and insulin resistance induced by fecal EVs of HFD-fed mice.

### Discussion of the EXAMPLES

Resistance to the biological effects of insulin is a hallmark of metabolic syndrome and an important contributing factor in the pathogenesis of type 2 diabetes. Here, for the first time is demonstrated that a HFD affects the composition of intestinal epithelial exosomes, an understudied intestinal community member or complex of gut metabolites. Indeed, intestinal exosomal PC from HFD-fed mice was sufficient for the development of insulin resistance in both SPF and germ-free mouse models. These effects of exosomes were abrogated by depleting exosomal PC, suggesting that insulin resistance in humans could be mediated by diet-induced changes to intestinal exosomal lipids.

It was observed that the percentage of intestinal exosomal PC was increased in exosomes from HFD-fed mice and type 2 diabetes patients, and the role of exosomal PC in developing insulin resistance was further demonstrated in a mouse model. Overproduction of PC contributes to human metabolic disorders, and reducing PC levels in the liver can lead to protection against the development of hepatic insulin resistance. Moreover, the level of PC in liver is inversely related to insulin sensitivity in humans. Diet intervention in obese adults reduces PC in skeletal muscle, which leads to improvements in clinical outcomes and enhanced insulin sensitivity.

However, whether increased exosomal PC contributes to the development of insulin resistance is not known. It was determined that the diet-dependent increase in exosomal PC is clinically applicable because abnormally high and abnormally low levels of PC in various tissues can influence energy metabolism and has been linked to disease progression. The molar ratio between PC and PE is a key determinant of liver health. Changes in the hepatic PC/PE molar ratio have been linked to development of non-alcoholic fatty liver disease (NAFLD) in humans, as well as liver failure, impaired liver regeneration, and the severity of alcoholic fatty liver disease. Thus, exosomal PC levels could be used as a diagnostic biomarker fortype 2 diabetes and metabolic related liver disease.

PEMT converts PE to PC and is found in the endoplasmic reticulum and in mitochondria-associated membranes. Mice lacking PEMT have lower levels of PC and are protected from HFD-induced obesity and insulin resistance. The results presented herein indicated that increased PC in the intestinal exosomes of mice fed a HFD was associated with an increased level of PEMT. These findings provided a rationale for further identifying the diet-derived factors that may modulate PEMT expression.

Unlike the biological effect of the free form of PC, which has no targeting specificity and is predominantly concentrated in the ER of PC producer cells, that PC carried by exosomes were targeted and delivered to specific recipient cell types was demonstrated herein. For H-Exo, PC-enriched exosomes were preferentially taken up by liver macrophages and had additive effects with macrophage-released cytokines, including TNF-α and IL-6, with respect to inhibiting glucose uptake by hepatocytes. H-Exo PC interacted with the AhR receptor in the recipient cells. Indeed, the data presented herein suggested that PC interacted with AhR and inhibited the expression of a number of genes involved in the insulin signaling pathway, including IRS2, an essential gene for insulin-mediated glucose uptake.

The liver has evolved exquisite sensing mechanisms to detect signals from the gut community through the gut/liver axis. While not wishing to be bound by any particular theory of operation, that AhR could be one such biosensor expressed in hepatocytes is supported by the data presented herein. Upon liver AhR-mediated detection of gut metabolites, such as exosomes, activated AhR cross-talks with transcriptional factors to rewire liver cell metabolism and reprogram the cellular transcriptome. Depending on what type of ligands bind to AhR, different transcription factors could selectively crosstalk with each other. The results presented herein demonstrated that HFD-induced exosomal PC bound to AhR in hepatocytes and subsequently inhibited IRS-2-mediated glucose uptake. This reaction could be one intestinal exosome-mediated feedback mechanism to protect the liver from over depositing energy generated from a HFD.

As also disclosed herein, H-Exo-induced insulin resistance was gut microbiome independent. HFD altered the composition and function of intestinal exosomes. HFD is known to cause gut dysbiosis. Therefore, the data presented herein established a foundation for further studying whether dysbiosis of the gut microbiota alters the composition and function of intestinal exosomes. Also, future investigations can determine whether the composition of intestinal exosomes can be altered by other diets and whether the lipid profile of exosomes can be manipulated by a given healthy diet to improve insulin sensitivity.

Overall, the presently disclosed results support a model wherein intestinal exosomes play a role in mediating gut/liver communication. Diet can alter the composition and therefore the function of intestinal exosomes. Insulin resistance can be induced in an exosome PC-dependent manner by activation of the AHR receptor-mediated pathway. The results presented herein supported additional research examining whether gut community-derived factors that can prevent PC recruitment into intestinal exosomes may be useful in the prevention or treatment of type 2 diabetes in humans.

Regarding insulin resistance, insulin resistance is a hallmark of obesity, being a forerunner of type 2 diabetes and metabolic syndrome. A high-fat diet has been associated with insulin sensitivity. How a high-fat diet causes insulin resistance has remained a mystery. Presented herein are data showing that lean mice become insulin resistant after being administered exosomes isolated from the feces of obese mice fed a high-fat diet (HFD) or from human type II diabetic patients. A high-fat diet (HFD) altered the lipid composition of exosomes, such that the lipid profile switched from predominantly PE in exosomes from lean animals (L-Exo) to PC in exosomes from obese animals (H-Exo). Treatment with PC-depleted exosomes did not cause glucose intolerance or insulin resistance.

Mechanistically, disclosed herein is evidence that intestinal H-Exo trafficked to the liver and were subsequently taken up predominantly by macrophages and hepatocytes, leading to inhibition of the insulin signaling pathway. The supernatants from cultures of exosome⁺ macrophages had an additive effect with PC on the inhibition of glucose uptake by hepatocytes. Moreover, it was determined that exosome-derived PC bound to and activated hepatic AhR, leading to inhibition of the expression of genes essential for activation of the insulin signaling pathway, including IRS-2. Together, the presently disclosed subject matter results revealed an important role for intestinal exosomes in gut-liver communication, and HFD-induced exosomes were identified as contributors to the development of insulin resistance. Intestinal exosomes thus have potential as broad therapeutic targets, and indeed inhibiting diet-induced alterations to their lipid compositions is shown to be of value. The human intestinal lumen contains numerous metabolites that accumulate in the bloodstream, where they can have systemic effects on the host. Intestinal epithelial exosomes are released into the intestinal lumen. From an obese mouse model and type II diabetes feces, exosomes were isolated that were capable of transferring insulin resistance to lean mice. A high fat diet altered the lipid composition of exosomes from PE to PC enriched exosomes. Depletion of exosomal PC led to restoring glucose tolerance and insulin response. Mechanistically, it was shown that intestinal exosomes trafficked to the liver and subsequently were up taken by hepatocytes and macrophages, leading to inhibition of activation of insulin signaling pathway. The supernatants from exosomes+macrophages had an additive effect on the inhibition of glucose up taken by hepatocytes. Exosomes PC binds to hepatic AHR, leading to nuclear translocation of AHR which inhibits the expression of genes that are essential for activation of insulin pathway.

A HFD is known to change cellular physiology and lead to the development of detrimental health outcomes such as obesity or T2D. Studies on mice and humans have suggested that chronic consumption of a HFD causes inactivation of the transcription factor Foxa2. Studies have further suggested that activated Foxa2 promotes insulin signaling, while AhR overexpression inhibits the insulin response. However, it is unclear whether there is a link between Foxa2 and AhR transcription factors that regulate insulin homeostasis, or whether diet-derived factors regulate the potential communication between Foxa2 and AhR.

Here, for the first time, it is disclosed that ginger-derived nanoparticles (GDNP) can prevent insulin resistance by restoring homeostasis in gut epithelial Foxa2/AhR signaling in mice fed the HFD. Foxa2 inhibits AhR expression by induction of miR-375 which targets AhR, whereas AhR inhibits the expression of Foxa2 by bacterial indole mediated activation of the AhR pathway. It is further shown that a HFD disrupts this balance by over-activating the AhR-mediated signaling pathway. Once AhR is activated by its ligand, it inhibits the expression of IRS-1 and -2 amplifying the action of insulin in insulin-sensitive tissues.

Foxa2 mediated induction of miR-375 inhibits AhR expression via a potential binding site in the AhR 3′ UTR. This process occurs in a tightly regulated manner, as evidenced by the fact that GDNP treatment not only induced the expression of miR-375 but also of VAMP7, which monitors intracellular levels of miR-375. When intestinal epithelial cells received a GDNP dose higher than 1×10⁶ GDNPs/ml, miR-375 was sorted into exosomes in a VAMP7-dependent manner to reduce the intracellular level of miR-375 and prevent further reduction of AhR. These findings provide a foundation for future studies to determine the cellular machinery that monitors the level of intracellular miR-375 and AhR which underlie the timing for sorting of miR-375 into exosomes in a GDNP dose-dependent manner.

Intestinal epithelial miRNAs released into the lumen have been suggested to modulate the composition and function of gut microbiota. The present disclosure shows that exosomal miR-375 targets the bacterial tnaA gene and ultimately inhibits the production of indole, a known AhR ligand. Moreover, the present disclosure indicates that indole activation of the AhR pathway inhibited the expression of Foxa2. GDNP treatment led to decreased indole production by increasing exosomal miR-375. As a result, AhR over-activation was prevented, and AhR homeostasis was maintained.

Moreover, GDNP treatment also restored the Foxa2/AhR equilibrium that was disrupted by the HFD by preventing AKT1 mediated phosphorylation of Foxa2. The finding that the insulin response is regulated by bidirectional communication between Foxa2 and AhR via diet-derived factors may have broader implications. Foxa2 is a pioneer transcription factor that has been found to play important roles in multiple stages of mammalian life, beginning with early development, continuing during organogenesis, and finally in regulating metabolism and glucose homeostasis in adults. Additionally, AhR has multiple ligands, including endogenous metabolites, nutrients and factors released by gut microbiota. Ligand-dependent activation of AhR can result in an extremely diverse spectrum of biological effects that occur in a ligand-, species- and tissue-specific manner.

The data presented herein support a model in which GDNP regulates the equilibrium of Foxa2/AhR in the gut milieu. In a healthy, varied diet, multiple particles of variable size and composition are ingested, each of which has a distinct effect on the regulation of Foxa2 and AhR activity. In summary, our findings support further exploration of the development of edible nanoparticle-based strategies for the prevention and treatment of metabolic disease.

The finding that miR-375 can be delivered via nano-vectors made up of GDNP lipids to target the E. coli tnaA gene and the hepatocyte AhR gene, the development of carriers to deliver miR-375 as a therapeutic orally to targeted tissues would represent a significant advance in the treatment of disease. In addition, this strategy would likely have few side effects, because the system is based on edible plant carriers to deliver therapeutic agents to the gut bacteria and liver.

### REFERENCES

All references listed in the instant disclosure, including but not limited to all patents, patent applications and publications thereof, scientific journal articles, and database entries are incorporated herein by reference in their entireties to the extent that they supplement, explain, provide a background for, or teach methodology, techniques, and/or embodiments employed herein.

It will be understood that various details of the presently disclosed subject matter can be changed without departing from the scope of the presently disclosed subject matter. Furthermore, the foregoing description is for the purpose of illustration only, and not for the purpose of limitation.

1. A method for increasing insulin sensitivity in a subject in need thereof, the method comprising administering to the subject an effective amount of a composition comprising a lipid bilayer, wherein the lipid bilayer is low in total phosphatidylcholine (PC) or has been treated to reduce total PC.

2. The method of claim 1, wherein the composition is a nanoparticle or an exosome, optionally an intestinal exosome, further optionally an intestinal exosome isolated from the subject.

3. The method of claim 1, wherein the composition is a ginger-derived nanoparticle (GDNP).

4. The method of claim 1, wherein the lipid bilayer of the composition has a total PC content that does not exceed about 14% lysophosphatidylcholine (LPC), about 10% ether-phosphatidylcholine (ePC), and/or about 10% PC as compared to total lipids.

5. A method for diagnosing insulin sensitivity and/or a metabolic-related disorder of the liver in a subject, the method comprising assaying total phosphatidylcholine (PC) of intestinal exosomes isolated from the subject, wherein a total PC content of the intestinal exosomes isolated from the subject that is elevated relative to intestinal exosomes isolated from a normal subject is indicative of insulin sensitivity an/or a metabolic-related disorder of the liver in the subject.

6. A method for identifying a subject with insulin sensitivity and/or a metabolic-related disorder of the liver, the method comprising assaying total phosphatidylcholine (PC) of intestinal exosomes isolated from the subject, wherein a total PC content of the intestinal exosomes isolated from the subject that is elevated relative to intestinal exosomes isolated from a normal subject is indicative of the subject having insulin sensitivity and/or a metabolic-related disorder of the liver.

7. The method of claim 6, wherein total PC content of the intestinal exosomes isolated from the subject that exceeds about 14% lysophosphatidylcholine (LPC), about 10% ether-phosphatidylcholine (ePC), and/or about 10% PC as compared to total lipids is indicative of the subject having insulin sensitivity and/or a metabolic-related disorder of the liver.

8. A method for preferentially targeting hepatocytes in a subject, the method comprising administering to the subject a composition comprising a lipid bilayer, optionally a nanoparticle, with a low total PC content and/or enhanced total phosphatidylethanolamine (PE) content, wherein the composition preferentially targets the subject's hepatocytes.

9. The method of claim 8, wherein the total PE content of the lipid bilayer comprises PE of at least 50%, ether-phosphoethanolamine (ePE) of at least 30%, or both.

10. The method of claim 8, wherein the composition is a nanoparticle or an exosome, optionally an intestinal exosome, and further optionally an intestinal exosome isolated from the subject.

11. The method of claim 8, wherein the exosome is an intestinal exosome that has been treated to reduce the total PC content to less than about 35% and/or to enhance the total PE content to greater than about 35%.

12. A method for preferentially targeting liver macrophages and/or monocytes in a subject, the method comprising administering to the subject a composition comprising a lipid bilayer, optionally a nanoparticle, with a high total PC content and/or a reduced total PE content, wherein the composition preferentially targets the subject's liver macrophages and/or monocytes.

13. The method of claim 13, wherein the total PE content of the lipid bilayer comprises PE of less than 35%, ether-phosphoethanolamine (ePE) of less than 30%, or both.

14. The method of claim 12, wherein the composition is a nanoparticle or an exosome, optionally an intestinal exosome, and further optionally an intestinal exosome isolated from the subject.

15. The method of claim 12, wherein the exosome is an intestinal exosome that has a total PC content greater than about 35% and/or a total PE content of less than about 35%.

16. A method for inhibiting development of insulin resistance, optionally insulin resistance associated with diabetes, in a subject in need thereof, the method comprising administering to the subject a ginger-derived nanoparticle (GDNP) in an amount and via a route sufficient to inhibit development of insulin resistance in the subject.

17. The method of claim 16, wherein the GDNP is administered to the subject orally.

18. The method of claim 16, wherein the development of insulin resistance is incident to a high fat diet consumed by the subject.

19. A method for restoring homeostasis in gut epithelium in a subject in need thereof, the method comprising administering to the subject a ginger-derived nanoparticle (GDNP) in an amount and via a route sufficient to restore homeostasis in gut epithelium in the subject.

20. A method for enhancing expression of a Foxa2 gene product in a cell, the method comprising contacting the cell with a ginger-derived nanoparticle (GDNP) in an amount sufficient to enhance expression of the Foxa2 gene product in the cell.

21. A method for inhibiting Akt-1-mediated inactivation of a Foxa2 biological activity in a subject, the method comprising administering to the subject a ginger-derived nanoparticle (GDNP) in an amount and via a route sufficient to inhibit Akt-1-mediated inactivation of a Foxa2 biological activity in the subject.

22. A method for increasing expression of VAMP7, miR-375, or both in an epithelial cell, optionally an epithelial cell present in a subject, the method comprising contacting the epithelial cells with a ginger-derived nanoparticle (GDNP) in an amount sufficient to increase expression of VAMP7, miR-375, or both in the epithelial cell.

23. A method for enhancing sorting of miR-375 from intestinal epithelial cells to exosomes, the method comprising contacting the intestinal epithelial cells with a ginger-derived nanoparticle (GDNP) in an amount sufficient to enhance sorting of miR-375 from the intestinal epithelial cells to exosomes.

24. The method of claim 23, wherein the intestinal epithelial cells are present in a subject.

25. A method for inhibiting hepatic AhR expression in a subject, the method comprising administering to the subject a ginger-derived nanoparticle (GDNP) in an amount and via a route sufficient to enhance sorting of miR-375 from intestinal epithelial cells to exosomes in the subject, whereby the exosomes are taken up by hepatocytes in the subject in an amount sufficient to inhibit hepatic applicants hereby reserve expression in the subject.

26. A method for inhibiting development of obesity in a subject in need thereof, the method comprising administering to the subject a ginger-derived nanoparticle (GDNP) in an amount and via a route sufficient to inhibit development of obesity in the subject.

27. The method of claim 1, wherein the subject or the cell is a human subject or a human cell.


SEQUENCE LISTING (35 sequences)

SEQ ID NO: 1 - DNA, 21 bp, Artificial Sequence
  Artificially synthesized oligonucleotide
gcaatagctactccacttcag

SEQ ID NO: 2 - DNA, 20 bp, Artificial Sequence
  Artificially synthesized oligonucleotide
ggtgtgaagtctagcttgtg

SEQ ID NO: 3 - DNA, 19 bp, Artificial Sequence
  Artificially synthesized oligonucleotide
gtccaggcactggagcttt

SEQ ID NO: 4 - DNA, 20 bp, Artificial Sequence
  Artificially synthesized oligonucleotide
gctggtagcgcttcactctt

SEQ ID NO: 5 - DNA, 21 bp, Artificial Sequence
  Artificially synthesized oligonucleotide
tgacatccgcaacgactatca

SEQ ID NO: 6 - DNA, 23 bp, Artificial Sequence
  Artificially synthesized oligonucleotide
ccagtgcgtagttgtagaagagt

SEQ ID NO: 7 - DNA, 19 bp, Artificial Sequence
  Artificially synthesized oligonucleotide
gtgctgcatcgctgcttac

SEQ ID NO: 8 - DNA, 20 bp, Artificial Sequence
  Artificially synthesized oligonucleotide
cggtccgaacagacaaactg

SEQ ID NO: 9 - DNA, 20 bp, Artificial Sequence
  Artificially synthesized oligonucleotide
tcagacgaacaaggctgtcc

SEQ ID NO: 10 - DNA, 22 bp, Artificial Sequence
  Artificially synthesized oligonucleotide
ccatctaggcaatctcggtctc

SEQ ID NO: 11 - DNA, 21 bp, Artificial Sequence
  Artificially synthesized oligonucleotide
tgctctcgtgatgcttggttt

SEQ ID NO: 12 - DNA, 21 bp, Artificial Sequence
  Artificially synthesized oligonucleotide
atccacgtaattcgaggcttg

SEQ ID NO: 13 - DNA, 19 bp, Artificial Sequence
  Artificially synthesized oligonucleotide
ttcctccagtccgagagcg

SEQ ID NO: 14 - DNA, 21 bp, Artificial Sequence
  Artificially synthesized oligonucleotide
tgagaaggtccgagttcttgg

SEQ ID NO: 15 - DNA, 21 bp, Artificial Sequence
  Artificially synthesized oligonucleotide
ccctacgccaacatgaactcg

SEQ ID NO: 16 - DNA, 20 bp, Artificial Sequence
  Artificially synthesized oligonucleotide
gttctgccggtagaaaggga

SEQ ID NO: 17 - DNA, 23 bp, Artificial Sequence
  Artificially synthesized oligonucleotide
tcaagagcacagacagcacttcc

SEQ ID NO: 18 - DNA, 24 bp, Artificial Sequence
  Artificially synthesized oligonucleotide
gccatgtaaatccaccacagagag

SEQ ID NO: 19 - DNA, 25 bp, Artificial Sequence
  Artificially synthesized oligonucleotide
ccaagaaagtatggacacagacaaa

SEQ ID NO: 20 - DNA, 22 bp, Artificial Sequence
  Artificially synthesized oligonucleotide
gcattcttgatccttccttggt

SEQ ID NO: 21 - DNA, 22 bp, Artificial Sequence
  Artificially synthesized oligonucleotide
gctccgcctccatgagtcaata

SEQ ID NO: 22 - DNA, 20 bp, Artificial Sequence
  Artificially synthesized oligonucleotide
cacgcgagccgaacgaacaa

SEQ ID NO: 23 - DNA, 28 bp, Artificial Sequence
  Artificially synthesized oligonucleotide
gtacgcgtcccacatgtgttcaccagca

SEQ ID NO: 24 - DNA, 24 bp, Artificial Sequence
  Artificially synthesized oligonucleotide
gaggtaccccggagcggaagaccc

SEQ ID NO: 25 - DNA, 41 bp, Artificial Sequence
  Artificially synthesized oligonucleotide
gtgtgctccgcctccacaagccacgatttgccccgagcaaa

SEQ ID NO: 26 - DNA, 41 bp, Artificial Sequence
  Artificially synthesized oligonucleotide
tttgctcggggcaaatcgtggcttgtggaggcggagcacac

SEQ ID NO: 27 - DNA, 19 bp, Artificial Sequence
  Artificially synthesized oligonucleotide
tgcaaccatcaccagtaac

SEQ ID NO: 28 - DNA, 21 bp, Artificial Sequence
  Artificially synthesized oligonucleotide
gtccattaccaccggaatatc

SEQ ID NO: 29 - DNA, 26 bp, Artificial Sequence
  Artificially synthesized oligonucleotide
agcaactaaacaacctgccagtacta

SEQ ID NO: 30 - DNA, 21 bp, Artificial Sequence
  Artificially synthesized oligonucleotide
gtccggatattcaaggatgca

SEQ ID NO: 31 - DNA, 22 bp, Artificial Sequence
  Artificially synthesized oligonucleotide
tttgttcgttcggctcgcgtga

SEQ ID NO: 32 - DNA, 11 bp, Artificial Sequence
  Artificially synthesized oligonucleotide
atgagtcaata

SEQ ID NO: 33 - DNA, 11 bp, Artificial Sequence
  Artificially synthesized oligonucleotide
acaagccaacg

SEQ ID NO: 34 - RNA, 21 nt, Artificial Sequence
  Artificially synthesized oligonucleotide
agugcgcucgguugcuuguuu

SEQ ID NO: 35 - RNA, 22 nt, Artificial Sequence
  Artificially synthesized oligonucleotide
aaacaaguaauauagauauguu